(19) United States
(12) Patent Application Publication
(10) Pub. No.: US 2022/0249454 A1
(43) Pub. Date: Aug. 11, 2022

(54) INHIBITORS OF MONOCARBOXYLATE TRANSPORTERS FOR CANCER IMMUNOTHERAPY

(71) Applicant: NIROGY THERAPEUTICS, INC., Framingham, MA (US)

(72) Inventors: Vincent SANDANAYAKA, Northboro, MA (US); Gregory GORECZNY, Cambridge, MA (US); Sambard SHARMA, Framingham, MA (US)

(21) Appl. No.: 17/577,612

(22) Filed: Jan. 18, 2022

Related U.S. Application Data
(60) Provisional application No. 63/139,122, filed on Jan. 19, 2021.

(51) Int. Cl. A61K 31/4436; A61P 35/00; A61K 31/381; A61K 31/429; A61K 45/06

(52) U.S. Cl. CPC A61K 31/4436; A61P 35/00; A61K 45/06; A61K 31/429; A61K 31/381

(57) ABSTRACT

The present disclosure provides a method of treating cancer in a subject. The method including administering to the subject: a) a therapeutically effective amount of a monocarboxylate transporter (MCT) inhibitor alone; or in combination with b) a therapeutically effective amount of an immunotherapy agent, wherein the monocarboxylate transporter inhibitor is represent by Formula (I):

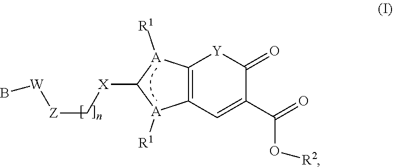

or a pharmaceutically acceptable salt thereof, wherein subscript n, B, W, X, Y, Z,

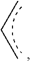

each A, each R¹, and R² are as provided herein. Also provided are a pharmaceutical composition thereof and a kit thereof for treating cancer in a subject.

### CROSS-REFERENCES TO RELATED APPLICATIONS

This application claims priority to U.S. Provisional Application No. 63/139,122 filed Jan. 19, 2021, which is incorporated in its entirety for all purposes.

### STATEMENT AS TO RIGHTS TO INVENTIONS MADE UNDER FEDERALLY SPONSORED RESEARCH AND DEVELOPMENT

Not Applicable

### REFERENCE TO A “SEQUENCE LISTING,” A TABLE, OR A COMPUTER PROGRAM LISTING APPENDIX SUBMITTED ON A COMPACT DISK

Not Applicable

### BACKGROUND OF THE INVENTION

It has been well demonstrated that tumors display altered cellular metabolism, in which cancer cells exhibit high rate of glucose consumption compared to the untransformed normal cells. Tumors contain well oxygenated (aerobic), and poorly oxygenated (hypoxic) regions. Compared to normal cells, some cancer cells are heavily dependent upon either aerobic glycolysis (Warburg effect, 1956) or anaerobic glycolysis (especially in hypoxic regions) for energy (ATP) production while maintaining a certain level of oxidative phosphorylation. This glycolytic switch by highly proliferating and hypoxic cancer cells provides the energy and biosynthetic needs for cancer cell survival. To maintain this metabolic phenotype, cancer cells up regulate a series of proteins, including glycolytic enzymes and pH regulators; monocarboxylate transporters (MCTs) that will facilitate the efflux of lactate co-transported with a proton. This fundamental difference between normal cells and cancer cells has not been previously applied to cancer therapy.

MCTs mediate influx and efflux of monocarboxylates such as lactate, pyruvate, ketone bodies (acetoacetate and beta-hydroxybutyrate) across cell membranes. These monocarboxylates play essential roles in carbohydrate, amino acid, and fat metabolism in mammalian cells, and must be rapidly transported across plasma membrane of cells. MCTs catalyze the transport of these solutes via a facilitative diffusion mechanism that requires co-transport of protons. Monocarboxylates such as lactate, pyruvate, and ketone bodies play a central role in cellular metabolism and metabolic communications among tissues. Lactate is the end product of aerobic glycolysis. Lactate has recently emerged as a critical regulator of cancer development, invasion, and metastasis. Tumor lactate levels correlate well with metastasis, tumor recurrence, and poor prognosis (J. Clin. Invest 2013).

MCTs are 12-span transmembrane proteins with N- and C-terminus in cytosolic domain and are members of solute carrier SLC16A gene family. MCT family contains 14 members, and so far MCT1, MCT2, MCT3, and MCT4 are well characterized [Biochemical Journal (1999), 343:281-299].

Malignant tumors contain aerobic and hypoxic regions, and the hypoxia increases the risk of cancer invasion and metastasis. Tumor hypoxia leads to treatment failure, relapse, and patient mortality as these hypoxic cells are generally resistant to standard chemo- and radiation therapy. In regions of hypoxia, cancer cells metabolize glucose into lactate whereas nearby aerobic cancer cells take up this lactate via the MCT1 for oxidative phosphorylation (OXPHOS). Under hypoxic conditions, cancer cells up regulate glucose transporters and consume large quantities of glucose. Cancer cells also up-regulate glycolytic enzymes and convert glucose into lactate, which is then efflux out of cell via MCT4. The nearby aerobic cancer cells take up this lactate via MCT1 for energy generation through OXPHOS. Thus, the limited glucose availability to the tumor is used most efficiently via synergistic metabolic symbiosis. This utilization of lactate as an energy substitute for survival prevents the aerobic cells from consuming large quantities of glucose.

MCTs are known to play central roles in many aspects of cancer biology. Cancer cells are highly glycolytic. Anaerobic glycolysis generates high intracellular lactate levels, requiring MCTs to pump lactate out of the cells to prevent intracellular acidosis and death. Interestingly, some cancer cells import lactate as the main fuel source to support their anabolic growth [Cancer (2018), 128: 2086-2103]. Furthermore, when lactate is pumped out of the cancer cells into the tumor microenvironment (TME), lactate is imported into immunocytes and incorporated into chromatin (lactylation) to reprogram gene expression [Immunity 2015, 3(3):435-449]. The resulting lactylation leads to the activation of immune suppressor cells and inhibition of effector T and NK cells, which collectively suppresses anti-tumor immunity [Science 2009, 324: 1029-1033]. High expression of MCTs in some cancers such as triple negative breast cancer (TNBC) is correlated with poor patient survival [Br. J. Cancer 2106, 114(3):256-261 and Cell Cycle 2016, 15(11):1462-1470]. Thus, MCT1 and MCT4 mediated import and export of lactate impacts two key cancer hallmarks: metabolism and immunity. Without being bond to a particular theory, it is believed that that dual inhibition of MCT1 and MCT4 would block influx and efflux of lactate, leading to cancer cell death and enhanced antitumor immunity. On the basis of preliminary data, it is believed that MCT inhibitors may represent a promising novel therapeutic approach for TNBC and other cancers by overcoming deficiencies of current chemo, targeted, and immunotherapy treatments.

In the realm of immune-oncology, therapies that inhibit the checkpoint mechanism induce responses by blocking the inhibitory mechanisms that control T-cell-mediated immunity. Immune checkpoints refer to the set of inhibitory pathways that immune cells possess in order to regulate and control the immune response. The main targets for therapeutics include cytotoxic T lymphocyte-associated antigen 4 (CTLA-4) receptor and programmed cell death 1 (PD-1) receptor or its ligand PD-L1, [Cancer Cell. 2017 Jun. 12; 31(6):848-848.e1, Immuno-Oncology. Prog Tumor Res. Basel, Karger, 2015, 42: 55-66, Molecules 2019].

PD-L1 is an immune co-regulatory molecule belonging to the B7 family [Nat Med 1999]. While PD-1 is predominantly expressed on activated T cells, PD-L1 is expressed on the surface of both cancer and immune cells. By triggering an inhibitory signal towards the T cell receptor-mediated activation, PD-L1 suppresses the proliferation, activation and infiltration of cytotoxic T-lymphocytes, consequently facilitating tumor immune escape and cancer progression [J Exp Med 2000, Nat Med 2002, Clin Immunol 2005]. Furthermore, PD-L1 has been reported to be aberrantly overexpressed in numerous types of tumor cell, including melanoma, ovarian and lung cancers, and patients with high PD-L1 expression are associated with unfavorable prognosis and significant risk of cancer-specific mortality [Eur J Cancer 2014, Clin Cancer Res 2009, Cancer 2010].

As MCT inhibition would impact the TME and induce activation of immune response, one might expect an effect on a broad range of cells involved in the immune system. These include the T-cells as well as B-cells, NK, NKT, macrophages, dendritic cells, etc.

There are small molecule compounds that appear to treat cancers by immune activation. The non-steroidal anti-inflammatory drug (NSAID) diclofenac and structural similar compounds lower lactate secretion of tumor cells and improves anti-PD1-induced T cell killing in vitro. Diclofenac is an inhibitor of the lactate transporters, monocarboxylate transporter 1 and 4 (IC₅₀=1.4 uM and 0.14 uM, respectively) and diminishes lactate efflux and impacts tumor glycolysis. However, high concentrations of drug (150-200 uM) are required to see efficacy in in vitro models. [Cell Reports 2019, 29:135-150]. At high concentrations, diclofenac may be engaging other targets in addition to MCTs. Indeed, diclofenac, which is a potent inhibitor of COX-2 and prostaglandin E2 synthesis, displays a range of effects on the immune system, the angiogenic cascade, chemo- and radio-sensitivity and tumor metabolism with pre-clinical and clinical evidence of these effects, in multiple cancer types [Ecancermedicalscience. 2016; 10: 610].

In preclinical studies, small molecule amide analogues of brefelamide have been found to suppress PD-L1 expression in different cancer cell lines and mitigated PD-1/PD-L1-mediated exhaustion of Jurkat T-lymphocytes co-cultured with A549 cells. It is postulated that the Hippo pathway is involved in the inhibition of PD-L1 by these compounds, which is mediated by a putative binding site for transcriptional co-activator with PDZ (TAZ)/Yes-associated protein (YAP)-TEA domain (TEAD) on PD-L1 promoter [Exp Therap Med 2020].

Syrosingopine, an anti-hypertensive drug, is a weak dual MCT1 and MCT4 inhibitor (IC₅₀=2.5 uM and 40 nM, respectively) that prevents lactate and H+ efflux. Syrosingopine elicits synthetic lethality in combination with the antidiabetic drug metformin, an inhibitor of mitochondrial NADH dehydrogenase. Syrosingopine treatment leads to high intracellular lactate levels and thereby end-product inhibition of lactate dehydrogenase. The loss of NAD+ regeneration capacity due to combined metformin and syrosingopine treatment resulted in glycolytic blockade, leading to ATP depletion and cell death. [Sci. Adv. 2016; 2: e1601756 and Cell Rep. 2018, 25(10:3047-3058]. Syrosingopine as a combination therapy with mitochondrial inhibitors has been patented in the context of cancer therapy and immunosuppression [U.S. Pat. No. 8,993,587]. However, syrosingopine has not been shown to have any in vivo anti-tumor activity in any mouse models.

### BRIEF SUMMARY OF THE INVENTION

In a first aspect, the present disclosure provides a method of treating cancer in a subject. The method includes administering to the subject:
a) a therapeutically effective amount of a monocarboxylate transporter (MCT) inhibitor alone; or in combination with
b) a therapeutically effective amount of an immunotherapy agent, wherein the monocarboxylate transporter inhibitor is represented by formula (I):

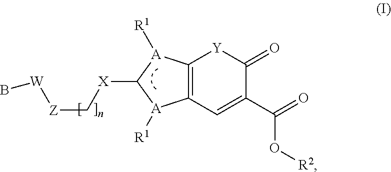

or a pharmaceutically acceptable salt thereof, wherein subscript n, B, W, X, Y, Z,

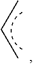

each A, each R¹, and R² are as provided herein.

In a second aspect, the present invention provides a pharmaceutical composition for treating cancer in a subject, the composition including:
a) a therapeutically effective amount of a monocarboxylate transporter (MCT) inhibitor; and
b) a therapeutically effective amount of an immune therapy agent, together with a pharmaceutically acceptable carrier or excipient,
   wherein the MCT inhibitor is represented by formula (I) as defined and described herein.

In a third aspect, the present invention provides a kit for treating cancer in a subject, the kit including:
a) a therapeutically effective amount of a monocarboxylate transporter (MCT) inhibitor; and
b) a therapeutically effective amount of an immune therapy agent, with a pharmaceutically acceptable carrier or excipient,
   wherein the MCT inhibitor is represented by formula (I) as defined and described herein.

### BRIEF DESCRIPTION OF THE DRAWINGS

FIGS. 1A-1B show the effects of Compound 1.009 alone and in combination with anti-CTLA-4 on mouse 4T1 tumor growth and survival. FIG. 1A: Growth kinetics of 4T1 tumors with the respective treatments at Day 39 of tumor growth. FIG. 1B: Kaplan-Meier Survival plot of 4T1 tumor growth.

FIGS. 2A-2E shows the effect of Compound 1.009 alone and in combination with anti-CTLA-4 on mouse spleen infiltrating lymphocytes and myeloid cells.

FIGS. 3A-3F show the effect of Compound 1.009 alone and in combination with anti-CTLA-4 on mouse tumor infiltrating lymphocytes and myeloid cells.

FIG. 4 shows the effects of Compound 1.009 alone and in combination with anti-PD-1 on mouse MC38 tumor growth.

### DETAILED DESCRIPTION OF THE INVENTION

### I. General

The present invention relates to the use of dual inhibitors of monocarboxylate transporters 1 and 4 alone or in combination with an immunotherapy agent (e.g., immune checkpoint inhibitors), chemotherapy, or other targeted therapies for the treatment of cancers. Also disclosed are pharmaceutical compositions including the monocarboxylate transporter inhibitor and the immunotherapy agent, together with a pharmaceutically acceptable carrier or excipient and a kit thereof for the treatment of cancers.

### II. Definitions

“Alkyl” refers to a straight or branched, saturated, aliphatic radical having the number of carbon atoms indicated (i.e., C₁₋₆ means one to six carbons). Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 20 carbons atoms, such as, but not limited to heptyl, octyl, nonyl, decyl, etc. Alkyl groups can be substituted or unsubstituted.

“Cycloalkyl” refers to a saturated or partially unsaturated, monocyclic, fused bicyclic or bridged polycyclic ring assembly containing from 3 to 12 ring atoms, or the number of atoms indicated. Cycloalkyl can include any number of carbons, such as C₃₋₆, C₄₋₆, C₅₋₆, C₃₋₈, C₄₋₈, C₅₋₈, C₆₋₈, C₃₋₉, C₃₋₁₀, C₃₋₁₁, and C₃₋₁₂. Saturated monocyclic cycloalkyl rings include, for example, cyclopropyl, cyclobutyl, cyclopentyl, cyclohexyl, and cyclooctyl. Saturated bicyclic and polycyclic cycloalkyl rings include, for example, norbornane, [2.2.2] bicyclooctane, decahydronaphthalene and adamantane. Cycloalkyl groups can also be partially unsaturated, having one or more double or triple bonds in the ring. Representative cycloalkyl groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene. When cycloalkyl is a saturated monocyclic C₃-C₈ cycloalkyl, exemplary groups include, but are not limited to cyclopropyl, cyclobutyl, cyclopentyl, cyclohexyl, cycloheptyl and cyclooctyl. When cycloalkyl is a saturated monocyclic C₃₋₆ cycloalkyl, exemplary groups include, but are not limited to cyclopropyl, cyclobutyl, cyclopentyl, and cyclohexyl. Cycloalkyl groups can be substituted or unsubstituted.

“Alkoxy” refers to an alkyl group having an oxygen atom that connects the alkyl group to the point of attachment: alkyl-O—. Alkoxy groups can have any suitable number of carbon atoms, such as C₁-C₆. Alkoxy groups include, for example, methoxy, ethoxy, propoxy, iso-propoxy, butoxy, 2-butoxy, iso-butoxy, sec-butoxy, tert-butoxy, pentoxy, hexoxy, etc.

“Halogen” or “halo” refers to fluorine, chlorine, bromine and iodine.

“Haloalkyl” refers to alkyl, as defined above, where some or all of the hydrogen atoms are replaced with halogen atoms. As for alkyl group, haloalkyl groups can have any suitable number of carbon atoms, such as C₁-C₆. For example, haloalkyl includes trifluoromethyl, fluoromethyl, 2,2,2-trifluoroethyl, etc. In some instances, the term “perfluoro” can be used to define a compound or radical where all the hydrogens are replaced with fluorine. For example, perfluoromethyl refers to 1,1,1-trifluoromethyl.

“Haloalkoxy” refers to an alkoxy group where some or all of the hydrogen atoms are substituted with halogen atoms. As for an alkyl group, haloalkoxy groups can have any suitable number of carbon atoms, such as C₁-C₆. The alkoxy groups can be substituted with 1, 2, 3, or more halogens. When all the hydrogens are replaced with a halogen, for example by fluorine, the compounds are per-substituted, for example, perfluorinated. Haloalkoxy includes, but is not limited to, trifluoromethoxy, 2,2,2,-trifluoroethoxy, perfluoroethoxy, etc.

“Heterocycle” or “heterocycloalkyl” refers to a saturated or partially unsaturated, monocyclic, bicyclic, or tricyclic ring system having from 3 to 12 ring members and from 1 to 4 heteroatoms of N, O and S. The heteroatoms can also be oxidized, such as, but not limited to, —S(O)— and —S(O)₂—. Heterocycloalkyl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heterocycloalkyl groups, such as 1, 2, 3, or 4, or 1 to 2, 1 to 3, 1 to 4, 2 to 3, 2 to 4, or 3 to 4. The heterocycloalkyl group can include groups such as aziridine, azetidine, pyrrolidine, piperidine, azepane, azocane, quinuclidine, pyrazolidine, imidazolidine, piperazine (1,2-, 1,3- and 1,4-isomers), oxirane, oxetane, tetrahydrofuran, oxane (tetrahydropyran), oxepane, thiirane, thietane, thiolane (tetrahydrothiophene), thiane (tetrahydrothiopyran), oxazolidine, isoxazolidine, thiazolidine, isothiazolidine, dioxolane, dithiolane, morpholine, thiomorpholine, dioxane, or dithiane. The heterocycloalkyl groups can also be fused to aromatic or non-aromatic ring systems to form members including, but not limited to, indoline. Heterocycloalkyl groups can be unsubstituted or substituted. For example, heterocycloalkyl groups can be substituted with C₁₋₆ alkyl or oxo (═O), among many others.

“Aryl” refers to an aromatic ring system having any suitable number of ring atoms and any suitable number of rings. Aryl groups can include any suitable number of ring atoms, such as, 6, 7, 8, 9, 10, 11, 12, 13, 14, 15 or 16 ring atoms, as well as from 6 to 10, 6 to 12, or 6 to 14 ring members. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl. Aryl groups can be substituted or unsubstituted.

“Heteroaryl” refers to a monocyclic or fused bicyclic or tricyclic aromatic ring assembly containing 5 to 16 ring atoms, where from 1 to 5 of the ring atoms are a heteroatom such as N, O or S. The heteroatoms can also be oxidized, such as, but not limited to, —S(O)— and —S(O)₂—. Heteroaryl groups can include any number of ring atoms, such as, 5 to 6, 5 to 8, 6 to 8, 5 to 9, 5 to 10, 5 to 11, or 5 to 12 ring members. Any suitable number of heteroatoms can be included in the heteroaryl groups, such as 1, 2, 3, 4, or 5, or 1 to 2, 1 to 3, 1 to 4, 1 to 5, 2 to 3, 2 to 4, 2 to 5, 3 to 4, or 3 to 5. Heteroaryl groups can have from 5 to 10 ring members and from 1 to 4 heteroatoms, from 5 to 8 ring members and from 1 to 4 heteroatoms, or from 5 to 8 ring members and from 1 to 3 heteroatoms, or from 5 to 6 ring members and from 1 to 4 heteroatoms, or from 5 to 6 ring members and from 1 to 3 heteroatoms. The heteroaryl group can include groups such as pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted.

The heteroaryl groups can be linked via any position on the ring. For example, pyrrole includes 1-, 2- and 3-pyrrole, pyridine includes 2-, 3- and 4-pyridine, imidazole includes 1-, 2-, 4- and 5-imidazole, pyrazole includes 1-, 3-, 4- and 5-pyrazole, triazole includes 1-, 4- and 5-triazole, tetrazole includes 1- and 5-tetrazole, pyrimidine includes 2-, 4-, 5- and 6-pyrimidine, pyridazine includes 3- and 4-pyridazine, 1,2,3-triazine includes 4- and 5-triazine, 1,2,4-triazine includes 3-, 5- and 6-triazine, 1,3,5-triazine includes 2-triazine, thiophene includes 2- and 3-thiophene, furan includes 2- and 3-furan, thiazole includes 2-, 4- and 5-thiazole, isothiazole includes 3-, 4- and 5-isothiazole, oxazole includes 2-, 4- and 5-oxazole, isoxazole includes 3-, 4- and 5-isoxazole, indole includes 1-, 2- and 3-indole, isoindole includes 1- and 2-isoindole, quinoline includes 2-, 3- and 4-quinoline, isoquinoline includes 1-, 3- and 4-isoquinoline, quinazoline includes 2- and 4-quinoazoline, cinnoline includes 3- and 4-cinnoline, benzothiophene includes 2- and 3-benzothiophene, and benzofuran includes 2- and 3-benzofuran.

Some heteroaryl groups include those having from 5 to 10 ring members and from 1 to 3 ring atoms including N, O or S, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, isoxazole, indole, isoindole, quinoline, isoquinoline, quinoxaline, quinazoline, phthalazine, cinnoline, benzothiophene, and benzofuran. Other heteroaryl groups include those having from 5 to 8 ring members and from 1 to 3 heteroatoms, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, and isoxazole. Some other heteroaryl groups include those having from 9 to 12 ring members and from 1 to 3 heteroatoms, such as indole, isoindole, quinoline, isoquinoline, quinoxaline, quinazoline, phthalazine, cinnoline, benzothiophene, benzofuran and bipyridine. Still other heteroaryl groups include those having from 5 to 6 ring members and from 1 to 2 ring atoms including N, O or S, such as pyrrole, pyridine, imidazole, pyrazole, pyrazine, pyrimidine, pyridazine, thiophene, furan, thiazole, isothiazole, oxazole, and isoxazole.

Some heteroaryl groups include from 5 to 10 ring members and only nitrogen heteroatoms, such as pyrrole, pyridine, imidazole, pyrazole, triazole, pyrazine, pyrimidine, pyridazine, triazine (1,2,3-, 1,2,4- and 1,3,5-isomers), indole, isoindole, quinoline, isoquinoline, quinoxaline, quinazoline, phthalazine, and cinnoline. Other heteroaryl groups include from 5 to 10 ring members and only oxygen heteroatoms, such as furan and benzofuran. Some other heteroaryl groups include from 5 to 10 ring members and only sulfur heteroatoms, such as thiophene and benzothiophene. Still other heteroaryl groups include from 5 to 10 ring members and at least two heteroatoms, such as imidazole, pyrazole, triazole, pyrazine, pyrimidine, pyridazine, triazine (1,2,3-, 1,2,4- and 1,3,5-isomers), thiazole, isothiazole, oxazole, isoxazole, quinoxaline, quinazoline, phthalazine, and cinnoline.

“Optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower haloalkyl, lower haloalkenyl, lower haloalkynyl, lower perhaloalkyl, lower perhaloalkoxy, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N3, SH, SCH₃, C(O)CH₃, CO₂CH₃, CO₂H, pyridinyl, thiophene, furanyl, lower carbamate, and lower urea. Where structurally feasible, two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

“Bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

“Composition” as used herein is intended to encompass a product comprising the specified ingredients in the specified amounts, as well as any product, which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

“Pharmaceutically acceptable excipient” refers to a substance that aids the administration of an active agent to and absorption by a subject. Pharmaceutical excipients useful in the present invention include, but are not limited to, binders, fillers, disintegrants, lubricants, coatings, sweeteners, flavors and colors. One of skill in the art will recognize that other pharmaceutical excipients are useful in the present invention.

“Combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

“Modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

“Modulator” refers to a composition that increases or decreases the level of a target molecule or the function of a target molecule or the physical state of the target of the molecule.

“Inhibition”, “inhibits” and “inhibitor” refer to a compound that prohibits or a method of prohibiting, a specific action or function.

“Therapeutically effective amount” refers to an amount of a compound or of a pharmaceutical composition useful for treating or ameliorating an identified disease or condition, or for exhibiting a detectable therapeutic or inhibitory effect. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Treat”, “treating” and “treatment” refer to any indicia of success in the treatment or amelioration of an injury, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation.

“Administering” refers to oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject.

“Patient” or “subject” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, the patient is human.

“Jointly therapeutically effective amount” as used herein means the amount at which the therapeutic agents, when given separately (in a chronologically staggered manner, especially a sequence-specific manner) to a warm-blooded animal, especially to a human to be treated, show an (additive, but preferably synergistic) interaction (joint therapeutic effect). Whether this is the case can be determined inter alia by following the blood levels, showing that both compounds are present in the blood of the human to be treated at least during certain time intervals.

“Synergistic effect” as used herein refers to an effect of at least two therapeutic agents: a monocarboxylate transporter (MCT) inhibitor as defined herein; and an immunotherapy agent as defined herein, which is greater than the simple addition of the effects of each drug administered by themselves. The effect can be, for example, slowing the symptomatic progression of a proliferative disease, such as cancer, particularly lung cancer, or symptoms thereof. Analogously, a “synergistically effective amount” refers to the amount needed to obtain a synergistic effect.

“A,” “an,” or “a(n)”, when used in reference to a group of substituents or “substituent group” herein, mean at least one. For example, where a compound is substituted with “an” alkyl or aryl, the compound is optionally substituted with at least one alkyl and/or at least one aryl, wherein each alkyl and/or aryl is optionally different. In another example, where a compound is substituted with “a” substituent group, the compound is substituted with at least one substituent group, wherein each substituent group is optionally different.

### III. Method

In a first aspect, the present invention provides a method of treating cancer in a subject. The method includes administering to the subject:
a) a therapeutically effective amount of a monocarboxylate transporter (MCT) inhibitor alone; or in combination with
b) a therapeutically effective amount of an immunotherapy agent,
   wherein the monocarboxylate transporter inhibitor is represented by formula (I):

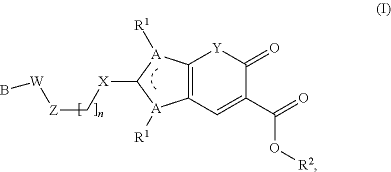

or a pharmaceutically acceptable salt thereof, wherein:
subscript n is 0, 1, or 2;
W is a bond, O, NH, or NR″;
X is O or NR″;
Y is O or NR″;
Z is a bond, CH₂, C═O, SO₂;

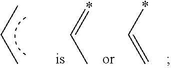

each A is independently selected from the group consisting of N, NR″, S, O, CR″ and CHR″;
each R¹ is independently absent or selected from the group consisting of hydrogen, halogen, C₁₋₆ alkyl, CHF₂, CF₃, CN, —C(O)R″, —C(O)OR″, —SO₂R″, —C(O)NR″₂, —C(O)N(OR″)R″, and —C≡CH;
R² is selected from the group consisting of:
   hydrogen;
   —C(O)R″;
   —(CH₂)₀₋₄C(O)R″;
   (CH₂)₀₋₄C(O)OR″;
   optionally substituted C₁₋₆ alkyl;
   an optionally substituted 3-8 membered saturated or partially unsaturated cycloalkyl ring;
   an optionally substituted 3-8 membered saturated or partially unsaturated heterocycloalkyl ring having 1-2 heteroatoms independently selected from nitrogen, oxygen, and sulfur;
   optionally substituted phenyl; and
   an optionally substituted 5-6 membered heteroaryl ring having 1-4 heteroatoms independently selected from nitrogen, oxygen, and sulfur;
B is a ring selected from the group consisting of:
   a 3-8 membered saturated or partially unsaturated monocyclic cycloalkyl ring, phenyl,
   a 8-10 membered bicyclic aryl ring,
   a 3-8 membered saturated or partially unsaturated heterocycloalkyl ring having 1-2 heteroatoms independently selected from nitrogen, oxygen, and sulfur,
   a 5-6 membered monocyclic heteroaryl ring having 1-4 heteroatoms independently selected from nitrogen, oxygen, and sulfur, and
   a 8-10 membered bicyclic heteroaryl ring having 1-4 heteroatoms independently selected from nitrogen, oxygen, and sulfur,
   wherein B is optionally substituted with one or more substituents selected from R¹, R′, and R″;
R′ is selected from the group consisting of OH, C₁₋₆ haloalkyl, C₁₋₆ alkoxy, C₁₋₆ haloalkoxy, and O-phenyl optionally substituted with halogen, C₁₋₆ alkyl, or C₁₋₆ alkoxy;

R″ is selected from the group consisting of hydrogen, C₁₋₆ alkyl, and C₁₋₆ haloalkyl; or selected from the group consisting of:
a 3-8 membered saturated or partially unsaturated cycloalkyl ring, optionally substituted with halogen or C₁₋₆ alkyl;
a 3-8 membered saturated or partially unsaturated heterocycloalkyl ring having 1-2 heteroatoms independently selected from nitrogen, oxygen, and sulfur, said ring optionally substituted with halogen or C₁₋₆ alkyl;
phenyl optionally substituted with halogen, C₁₋₆ alkyl, or C₁₋₆ alkoxy; and
a 5-6 membered heteroaryl ring having 1-4 heteroatoms independently selected from nitrogen, oxygen, and sulfur, said ring optionally substituted with halogen or C₁₋₆ alkyl.

In some embodiments, the present invention provides a method of treating cancer in a subject, the method including administering to the subject:
a) a therapeutically effective amount of a monocarboxylate transporter (MCT) inhibitor; and
b) a therapeutically effective amount of an immunotherapy agent,
   wherein the monocarboxylate transporter inhibitor is represented by formula (I) as defined and described herein.

In another aspect, the present invention provides a method of treating cancer in a subject. The method includes administering to the subject:
a) a therapeutically effective amount of a monocarboxylate transporter (MCT) inhibitor alone; or in combination with
b) a therapeutically effective amount of an immunotherapy agent,
   wherein the monocarboxylate transporter inhibitor is represented by the formula selected from the group consisting of:

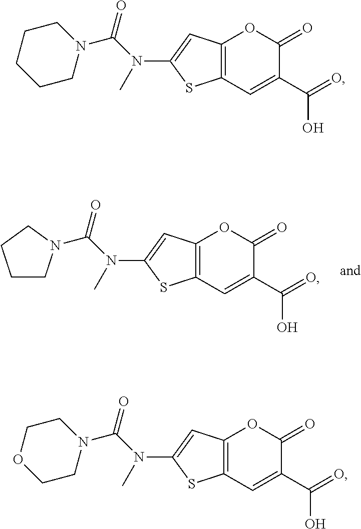 [SMILES: CN(C(=O)N1CCCCC1)c1cc2oc(=O)c(C(=O)O)cc2s1 ; CN(C(=O)N1CCCC1)c1cc2oc(=O)c(C(=O)O)cc2s1 ; CN(C(=O)N1CCOCC1)c1cc2oc(=O)c(C(=O)O)cc2s1]

or a pharmaceutically acceptable salt thereof.

The immunotherapy agent can be any one of therapy agents useful in immunotherapy.

In some embodiments, the immunotherapy agent is an immune checkpoint inhibitor, a chimeric antigen receptor (CAR) therapy agent, a vaccine, a modulator of myeloid cells or a macrophage, a modulator of NK cells, a modulator of one or more cytokines, or combinations thereof.

In some embodiments, the immunotherapy agent is an immune checkpoint inhibitor. In some embodiments, the immune checkpoint inhibitor is a PD-1/PD-L1 inhibitor. In some embodiments, the PD-1/PD-L1 inhibitor is pembrolizumab, nivolumab, atezolizumab, durvalumab, avelumab, cemiplimab-rwlc, camrelizumab, JS001, sintilimab, prolgolimab, tislelizumab, balstilimab, dostarlimab, or retifanlimab. In some embodiments, the immune checkpoint inhibitor is a CTLA-4 inhibitor. In some embodiments, the CTLA-4 inhibitor is ipilimumab or tremelimumab. In some embodiments, the CTLA-4 inhibitor is ipilimumab.

In some embodiments, the immunotherapy agent is a chimeric antigen receptor (CAR) therapy agent including CAR T-cells, CAR-macrophages, CAR-NK cells, or combinations thereof. In some embodiments, the chimeric antigen receptor (CAR) therapy agent includes CAR T-cells. In some embodiments, the chimeric antigen receptor (CAR) therapy agent includes CAR-macrophages. In some embodiments, the chimeric antigen receptor (CAR) therapy agent includes CAR-NK cells. In some embodiments, the CAR therapy agent is brexucabtagene, tisagenlecleucel, or axicabtagene ciloleucel.

In some embodiments, the immunotherapy agent is a modulator of myeloid cells or a macrophage. In some embodiments, the modulator of myeloid cells or the macrophage increases M1 macrophages and/or decreases M2 macrophages; or the modulator of myeloid cells or the macrophage decreases myeloid-derived suppressor cells and/or dendritic cells. In some embodiments, the modulator of myeloid cells or the macrophage increases M1 macrophages and/or decreases M2 macrophages. In some embodiments, the modulator of myeloid cells or the macrophage decreases myeloid-derived suppressor cells and/or dendritic cells. In some embodiments, the immunotherapy agent is a modulator of NK cells.

In some embodiments, the immunotherapy agent is a modulator of one or more cytokines (e.g., TGFbeta, IL-10, Arg-1, TNFalpha, IL-1beta, and IFN-gamma). In some embodiments, the modulator of one or more cytokines downregulates one or more of TGFbeta, IL-10, and Arg-1; or the modulator of one or more cytokines upregulates one or more of TNFalpha, IL-1beta, and IFN-gamma. In some embodiments, the modulator of one or more cytokines downregulates one or more of TGFbeta, IL-10, and Arg-1. In some embodiments, the modulator of one or more cytokines upregulates one or more of TNFalpha, IL-1beta, and IFN-gamma.

In some embodiments, the immunotherapy agent is a vaccine.

The MCT inhibitor as defined and described herein can block an expression of one or more immune checkpoint molecules. In some embodiments, the MCT inhibitor blocks an expression of one or more immune checkpoint molecules. In some embodiments, the one or more immune checkpoint molecules includes PD-1, CTLA-4, TIM-3, LAG-3, B7-H3, B7-H4, PD-L1, PD-L2, or combinations thereof in B7 protein family. In some embodiments, the one or more immune checkpoint molecules further includes ICOSL, OX40L, Galactin-9, or combinations thereof. In some embodiments, the MCT inhibitor blocks an expression of one or more immune checkpoint molecules including PD-1, CTLA-4, TIM-3, LAG-3, B7-H3, B7-H4, PD-L1, PD-L2, or combinations thereof in B7 protein family. In some embodiments, the MCT inhibitor blocks an expression of one or more immune checkpoint molecules including PD-1, CTLA-4, TIM-3, LAG-3, B7-H3, B7-H4, PD-L1, PD-L2, or combinations thereof in B7 protein family, and/or ICOSL, OX40L, Galactin-9, or combinations thereof. In some embodiments, the MCT inhibitor blocks an expression of PD-L1 and/or PD-L2. In some embodiments, the MCT inhibitor blocks an expression of one or more B7 proteins. In some embodiments, the MCT inhibitor blocks an expression of B7-H3 and/or B7-H4. In some embodiments, the MCT inhibitor further blocks an expression of one or more of ICOSL, OX40L, and Galactin-9.

The MCT inhibitor as defined and described herein can upregulate production of one of more of IFN-gamma, TNF-alpha, and IL-1beta. In some embodiments, the MCT inhibitor upregulates a production of one of more of IFN-gamma, TNF-alpha, and IL-1beta. In some embodiments, the MCT inhibitor upregulates a production of IFN-gamma.

The cancer can be characterized by a solid tumor or a liquid tumor. In some embodiments, the cancer comprises a solid tumor. In some embodiments, the cancer comprises a liquid tumor.

In some embodiments, the cancer is breast, melanoma, colorectal, lung, bladder, ovarian, cervical, brain, CNS, skin, pancreatic, gastrointestinal, liver, kidney, head and neck, prostate, osteosarcoma, or combinations thereof.

The cancer can also be any cancer that is resistant to the treatment of an immunotherapy agent as described herein. In some embodiments, the cancer is resistant to an immunotherapy agent (e.g., an immune checkpoint inhibitor, a CAR therapy agent, a vaccine, a modulator of myeloid cells or a macrophage, and a modulator of one or more cytokines). In some embodiments, the cancer is resistant to an immune checkpoint inhibitor as described herein (e.g., a PD-1/PD-L1 inhibitor). In some embodiments, the cancer is resistant to a CAR therapy agent as described herein (e.g., brexucabtagene, tisagenlecleucel, and axicabtagene ciloleucel). In some embodiments, the cancer is resistant to a modulator of myeloid cells or a macrophage as described herein. In some embodiments, the cancer is resistant to a modulator of one or more cytokines as described herein. In some embodiments, the cancer is characterized by intrinsic and/or acquired resistance to an immunotherapy agent as described herein.

The MCT inhibitor as described herein and the immunotherapy agent as described herein can be provided in jointly therapeutically effective amounts or in synergistically effective amounts, or each of which can be used at a dose less than when each is used alone. In some embodiments, the MCT inhibitor and the immunotherapy agent are provided in jointly therapeutically effective amounts. In some embodiments, the MCT inhibitor and the immunotherapy agent are provided in synergistically effective amounts. In some embodiments, the MCT inhibitor and the immunotherapy agent are each used at a dose less than when each is used alone.

The MCT inhibitor as described herein and the immunotherapy agent as described herein can be administered concomitantly or sequentially. In some embodiments, the MCT inhibitor and the immunotherapy agent are administered concomitantly. In some embodiments, the MCT inhibitor and the immunotherapy agent are administered in a pharmaceutical composition comprising the MCT inhibitor and the immunotherapy agent. In some embodiments, the MCT inhibitor and the immunotherapy agent are administered sequentially. In some embodiments, the MCT inhibitor is administered prior to the administration of the immunotherapy agent. In some embodiments, the MCT inhibitor is administered after the administration of the immunotherapy agent.

The MCT inhibitor as described herein and the immunotherapy agent as described herein can be administered orally. In some embodiments, the MCT inhibitor and/or the immunotherapy agent are administered orally.

In some embodiments, the subject is human. In some embodiments, the subject is under the care of a medical practitioner, such as a physician. In some embodiments, the subject has been diagnosed with the cancer. In some embodiments, the subject has relapsed. In some embodiments, the subject has previously entered remission. In some embodiments, the subject has previously undergone, is undergoing, or will undergo a monotherapy course of treatment. In some embodiments, the subject has previously undergone, is undergoing, or will undergo radiation therapy. In some embodiments, the subject has previously undergone, is undergoing, or will undergo immunotherapy. In some embodiments, the subject has previously undergone, is undergoing, or will undergo chemotherapy.

The MCT inhibitor of formula (I) is further described according to Section IV. Compounds. In some embodiments, the PTPN11 inhibitor of formula (I) is any one of embodiments as described in Section IV. Compounds.

In some embodiments, the MCT inhibitor is selected from the group consisting of:

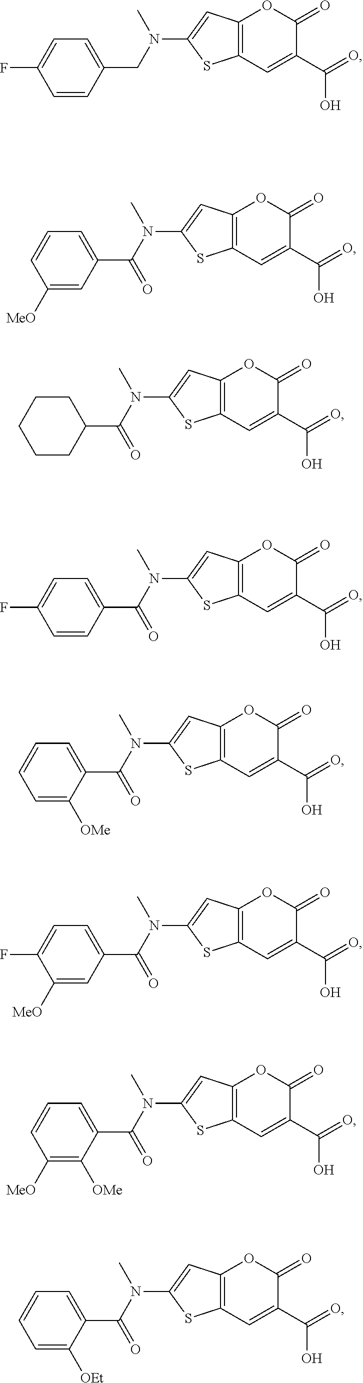

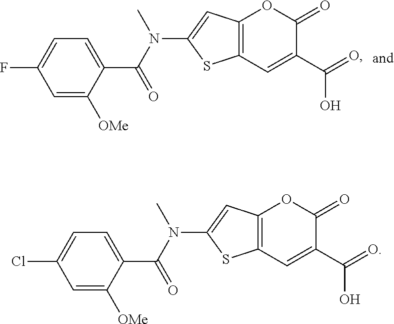 [SMILES: COc1cc(F)ccc1C(=O)N(C)c1cc2oc(=O)c(C(=O)O)cc2s1 ; COc1cc(Cl)ccc1C(=O)N(C)c1cc2oc(=O)c(C(=O)O)cc2s1]

In some embodiments, the MCT inhibitor is selected from the group consisting of:

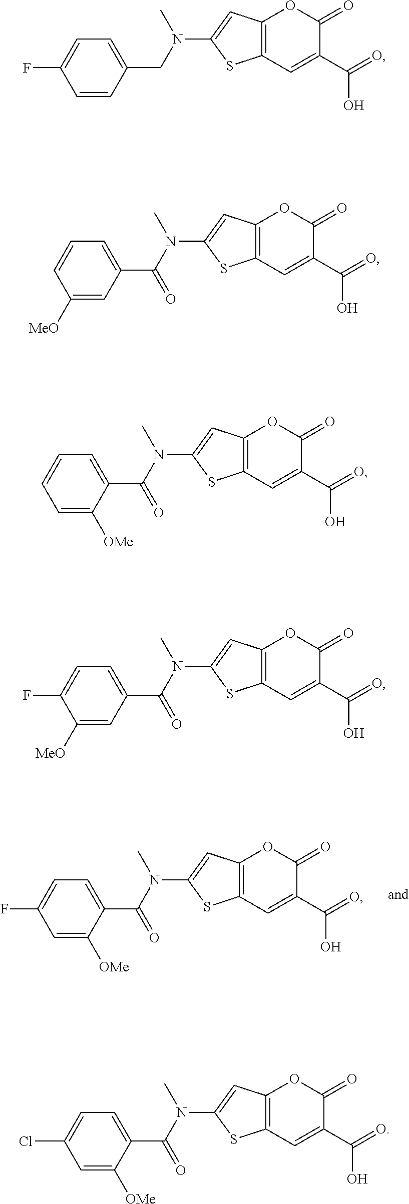 [SMILES: CN(Cc1ccc(F)cc1)c1cc2oc(=O)c(C(=O)O)cc2s1 ; COc1cccc(C(=O)N(C)c2cc3oc(=O)c(C(=O)O)cc3s2)c1 ; COc1ccccc1C(=O)N(C)c1cc2oc(=O)c(C(=O)O)cc2s1 ; COc1cc(C(=O)N(C)c2cc3oc(=O)c(C(=O)O)cc3s2)ccc1F ; COc1cc(F)ccc1C(=O)N(C)c1cc2oc(=O)c(C(=O)O)cc2s1 ; COc1cc(Cl)ccc1C(=O)N(C)c1cc2oc(=O)c(C(=O)O)cc2s1]

In some embodiments, the MCT inhibitor is selected from the group consisting of:

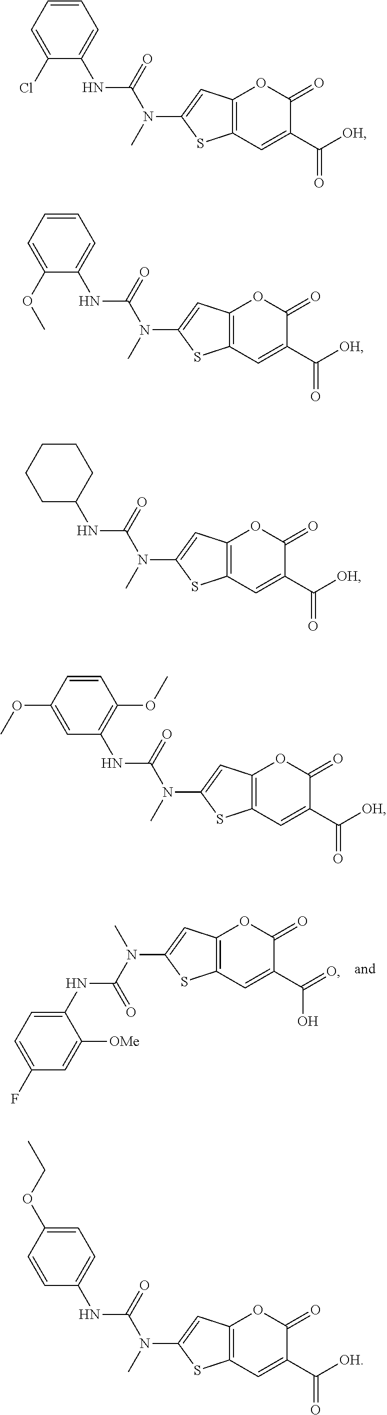

### IV. Compounds

The present disclosure provides a MCT inhibitor represent by formula (I) for use in a method of treating cancer in a subject as described in Section III: Method, a pharmaceutical composition for treating cancer in a subject as described in Section V: Composition; and a kit for treating cancer in a subject as described in Section VI: Kits. The MCT inhibitor is as defined and described in WO 2019/191599 and PCT/US2020/052413, the entirety of each of which is hereby incorporated for all purpose.

In some embodiments, the monocarboxylate transporter inhibitor is represented by formula (I):

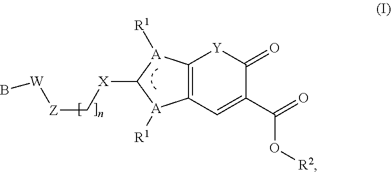

or a pharmaceutically acceptable salt thereof, wherein:
subscript n is 0, 1, or 2;
W is a bond, O, NH, or NR″;
X is O or NR″;
Y is O or NR″;
Z is a bond, CH₂, C═O, SO₂;

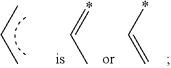

each A is independently selected from the group consisting of N, NR″, S, O, CR″ and CHR″;
each R¹ is independently absent or selected from the group consisting of hydrogen, halogen, C₁₋₆ alkyl, CHF₂, CF₃, CN, —C(O)R″, —C(O)OR″, —SO₂R″, —C(O)NR″2, —C(O)N(OR″)R″, and —C≡CH;
R² is selected from the group consisting of:
   hydrogen;
   —C(O)R″;
   —(CH₂)₀₋₄C(O)R″;
   —(CH₂)₀₋₄C(O)OR″;
   optionally substituted C₁-6 alkyl;
   an optionally substituted 3-8 membered saturated or partially unsaturated cycloalkyl ring;
   an optionally substituted 3-8 membered saturated or partially unsaturated heterocycloalkyl ring having 1-2 heteroatoms independently selected from nitrogen, oxygen, and sulfur;
   optionally substituted phenyl; and
   an optionally substituted 5-6 membered heteroaryl ring having 1-4 heteroatoms independently selected from nitrogen, oxygen, and sulfur;
B is a ring selected from the group consisting of:
   a 3-8 membered saturated or partially unsaturated monocyclic cycloalkyl ring, phenyl,
   a 8-10 membered bicyclic aryl ring,
   a 3-8 membered saturated or partially unsaturated heterocycloalkyl ring having 1-2 heteroatoms independently selected from nitrogen, oxygen, and sulfur,
   a 5-6 membered monocyclic heteroaryl ring having 1-4 heteroatoms independently selected from nitrogen, oxygen, and sulfur, and
   a 8-10 membered bicyclic heteroaryl ring having 1-4 heteroatoms independently selected from nitrogen, oxygen, and sulfur,
   wherein B is optionally substituted with one or more substituents selected from R′, R′, and R″;
R′ is selected from the group consisting of OH, C₁₋₆ haloalkyl, C₁₋₆ alkoxy, C₁₋₆ haloalkoxy, and O-phenyl optionally substituted with halogen, C₁₋₆ alkyl, or C₁₋₆ alkoxy;
R″ is selected from the group consisting of hydrogen, C₁₋₆ alkyl, and C₁₋₆ haloalkyl; or selected from the group consisting of:
   a 3-8 membered saturated or partially unsaturated cycloalkyl ring, optionally substituted with halogen or C₁₋₆ alkyl;
   a 3-8 membered saturated or partially unsaturated heterocycloalkyl ring having 1-2 heteroatoms independently selected from nitrogen, oxygen, and sulfur, said ring optionally substituted with halogen or C₁₋₆ alkyl;
   phenyl optionally substituted with halogen, C₁₋₆ alkyl, or C₁₋₆ alkoxy; and
   a 5-6 membered heteroaryl ring having 1-4 heteroatoms independently selected from nitrogen, oxygen, and sulfur, said ring optionally substituted with halogen or C₁₋₆ alkyl.

In some embodiments, when A is ═N—, S, O, NR″, ═CR′—, or CHR″, R¹ attached to the A is absent.

In some embodiments, one A is CR″ and the other A is S, provided each R¹ attached to each A is absent. In some embodiments, one A is CH and the other A is S, provided each R¹ attached to each A is absent.

In some embodiments of formula (I), W is a bond. In some embodiments of formula (I), W O, NH, or NR″.

In some embodiments, the compound of formula (I) is represented by formula (I-1):

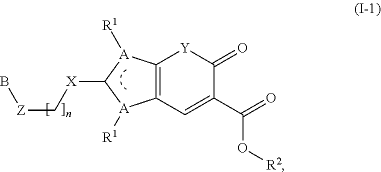

wherein subscript n, each A, B, X, Y, Z, each R¹, and R² are as defined and described herein.

In some embodiments, the compound of formula (I) is represented by formula (Ia):

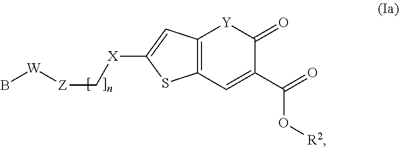

wherein subscript n, B, W, X, Y, Z, and R² are as defined and described herein.

In some embodiments, the compound of formula (I) is represented by formula (Ib):

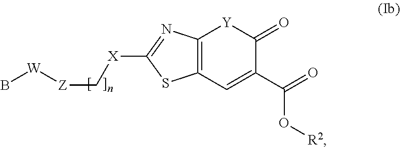

wherein subscript n, B, W, X, Y, Z, and R² are as defined and described herein.

In some embodiments of formula (Ia) or (Ib), W is a bond. In some embodiments of formula (Ia) or (Ib), W is O, NH, or NR″.

In some embodiments, the compound of any one of formulae (I), (I-1), and (Ia) is represented by formula (Ia-1):

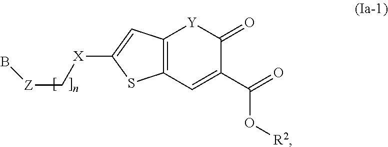

wherein subscript n, B, X, Y, Z, and R² are as defined and described herein.

In some embodiments, the compound of any one of formulae (I), (I-1), and (Ib) is represented by formula (Ib-1):

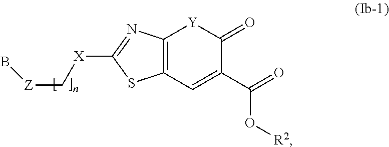

wherein subscript n, B, X, Y, Z, and R² are as defined and described herein.

With reference to any one of formulae (I), (I-1), (Ia), (Ia-1), (Ib), and (Ib-1), in some embodiments, subscript n is 0. In some embodiments, subscript n is 1.

With reference to any one of formulae (I), (I-1), (Ia), (Ia-1), (Ib), and (Ib-1), in some embodiments, X is NR″.

With reference to any one of formulae (I), (I-1), (Ia), (Ia-1), (Ib), and (Ib-1), in some embodiments, Y is O.

With reference to any one of formulae (I), (I-1), (Ia), (Ia-1), (Ib), and (Ib-1), in some embodiments, Z is a bond. In some embodiments, Z is CH₂. In some embodiments, Z is C═O. In some embodiments, Z is SO₂.

With reference to any one of formulae (I), (I-1), (Ia), (Ia-1), (Ib), and (Ib-1), in some embodiments, R² is hydrogen.

With reference to any one of formulae (I), (I-1), (Ia), (Ia-1), (Ib), and (Ib-1), in some embodiments, subscript n is 0; Y is O; and R² is hydrogen. In some embodiments, subscript n is 1; Y is O; and R² is hydrogen.

With reference to any one of formulae (I), (I-1), (Ia), (Ia-1), (Ib), and (Ib-1), in some embodiments, subscript n is 1 and Z is a bond; or subscript n is 0 and Z is CH₂. In some embodiments, subscript n is 0 and Z is C═O. In some embodiments, subscript n is 0 and Z is SO₂.

With reference to any one of formulae as described herein, in some embodiments, B is a ring selected from the group consisting of:
a 5-6 membered saturated or partially unsaturated monocyclic carbocyclic ring, phenyl,
a 8-10 membered bicyclic aryl ring,
a 5-8 membered saturated or partially unsaturated monocyclic or bicyclic heterocyclic ring having 1-2 heteroatoms independently selected from nitrogen, oxygen, and sulfur,
a 5-6 membered monocyclic heteroaryl ring having 1-4 heteroatoms independently selected from nitrogen, oxygen, and sulfur, and
a 8-10 membered bicyclic heteroaryl ring having 1-4 heteroatoms independently selected from nitrogen, oxygen, and sulfur,
   wherein B is optionally substituted with one or more substituents selected from R′, R′ and R″.

In some embodiments of B ring as defined and described herein, B is optionally substituted with one or more substituents selected from the group consisting of halogen, OH, CN, C₁₋₆ alkyl, C₁₋₆ haloalkyl, C₃₋₈ cycloalkyl, C₁₋₆ alkoxy, C₁₋₆ haloalkoxy, phenyl, and O-phenyl, wherein each phenyl is optionally independently substituted with halogen, C₁₋₆ alkyl, or C₁₋₆ alkoxy. In some embodiments, B is optionally substituted with one or more substituents selected from the group consisting of halogen, OH, CN, C₁₋₆ alkyl, C₁₋₆ haloalkyl, C₁₋₆ alkoxy, and C₁₋₆ haloalkoxy. In some embodiments, B is optionally substituted with one or more substituents selected from the group consisting of F, Cl, OH, CN, methyl, ethyl, isopropyl, tert-butyl, cyclopropyl, CF₃, OMe, OEt, OCF₃, and C(O)Me. In some embodiments, B is optionally substituted with one or more substituents selected from the group consisting of F, Cl, OH, CN, methyl, CF₃, OMe, OEt, and OCF₃.

In some embodiments, B ring is phenyl, optionally substituted with one or more substituents selected from R′, R′ and R″, wherein R′, R′ and R″ are as defined and described herein. In some embodiments, B ring is phenyl, optionally substituted with one or more substituents selected from the group consisting of halogen, OH, CN, C₁₋₆ alkyl, C₁₋₆ haloalkyl, C₃₋₈ cycloalkyl, C₁₋₆ alkoxy, C₁₋₆ haloalkoxy, and O-phenyl optionally substituted with halogen. In some embodiments, B is phenyl, optionally substituted with one or more substituents selected from the group consisting of halogen, OH, CN, C₁₋₆ alkyl, C₁₋₆ haloalkyl, C₁₋₆ alkoxy, and C₁₋₆ haloalkoxy. In some embodiments, B is phenyl, optionally substituted with one or more substituents selected from the group consisting of F, Cl, OH, CN, methyl, ethyl, isopropyl, tert-butyl, cyclopropyl, CF₃, OMe, OEt, OCF₃, and C(O)Me. In some embodiments, B is phenyl, optionally substituted with one or more substituents selected from the group consisting of F, Cl, OH, CN, methyl, CF₃, OMe, OEt, and OCF₃.

With reference to any one of formulae (I), (I-1), (Ia), (Ia-1), (Ib), and (Ib-1), in some embodiments, the compound is represented by the structure selected from the group consisting of:

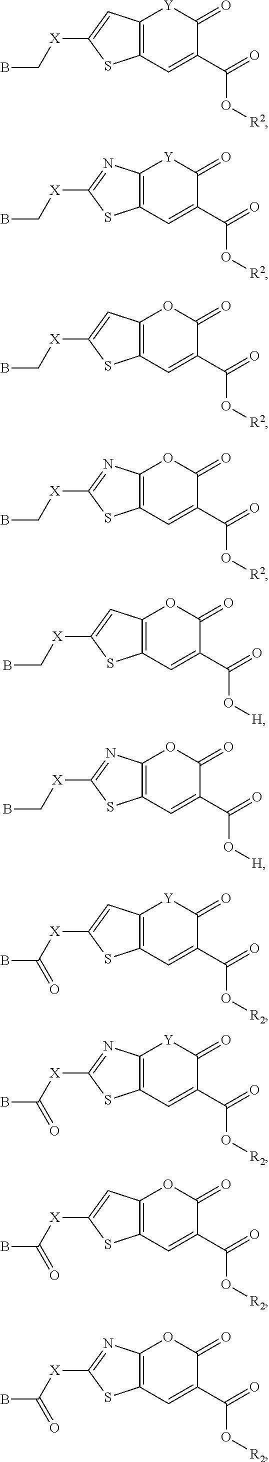

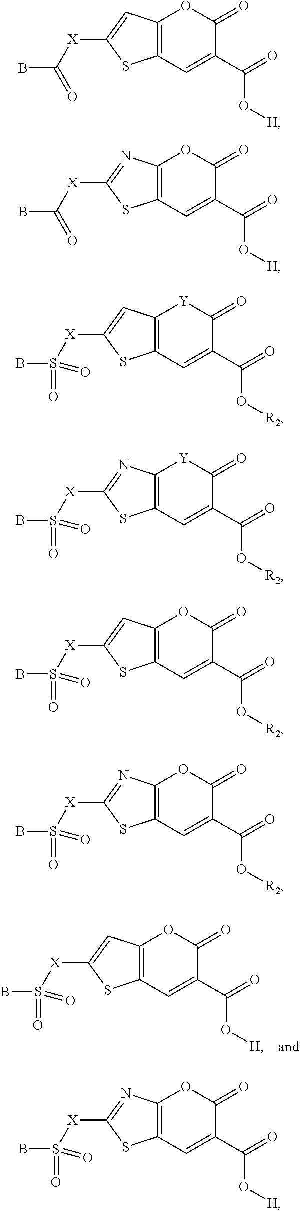

wherein B, X, Y, and R² are as defined and described herein.

In some embodiments of any one of formulae above, X is NR″. In some embodiments, Y is O. In some embodiments, R² is hydrogen. In some embodiments, X is NR″ and Y is O. In some embodiments, X is NR″; Y is O; and R² is hydrogen. In some embodiments, R″ is hydrogen. In some embodiments, R″ is C₁₋₆ alkyl. In some embodiments, R″ is methyl. In some embodiments, X is O, NH or NMe.

With reference to any one of formulae (I), (I-1), (Ia), (Ia-1), (Ib), and (Ib-1), in some embodiments, the compound is represented by the structure selected from the group consisting of:

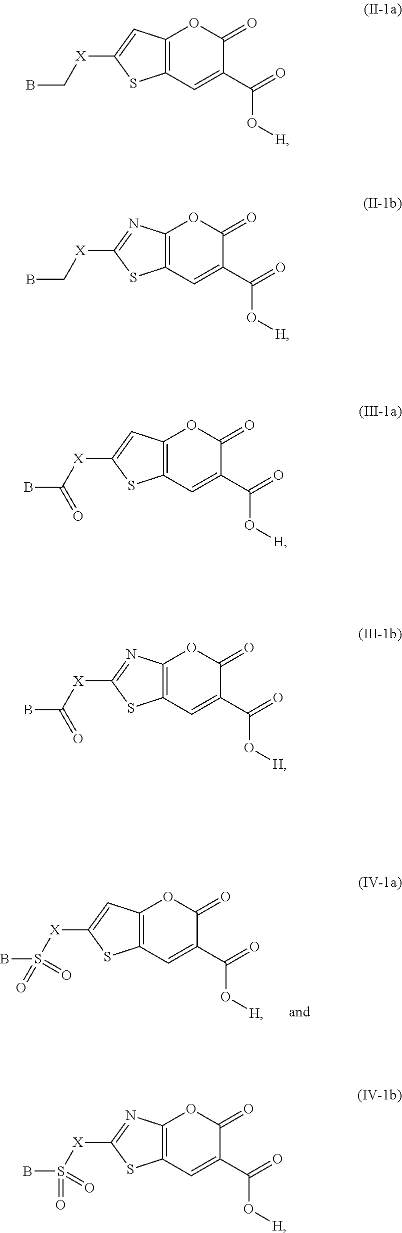

wherein B and X are as defined and described herein.

In some embodiments of any one of formulae (II-la) to (IV-1b), X is NR″. In some embodiments, R″ is hydrogen. In some embodiments, R″ is C₁₋₆ alkyl. In some embodiments, R″ is methyl. In some embodiments, X is O, NH or NMe.

In some embodiments of any one of formulae above, W is a bond and B is selected from the group consisting of:

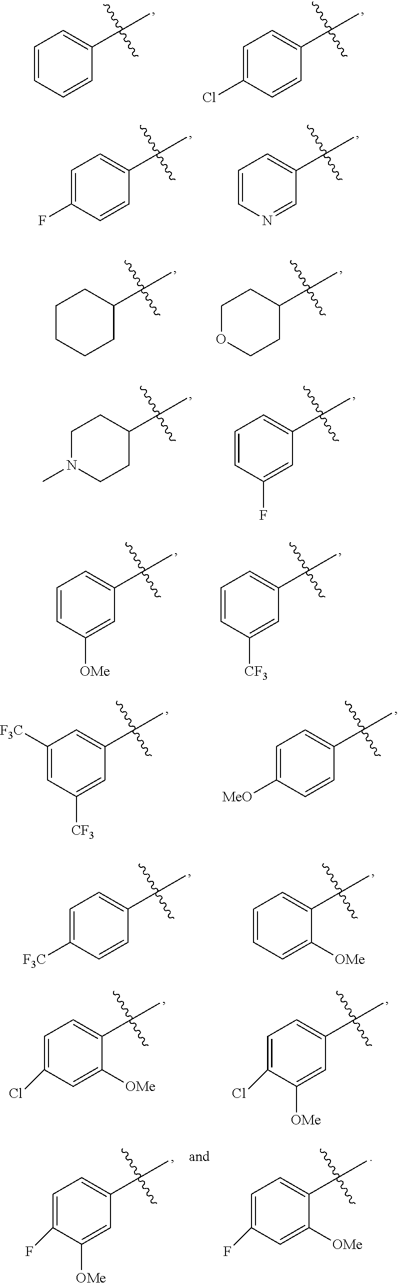

In some embodiments, the compound is selected from the group consisting of:

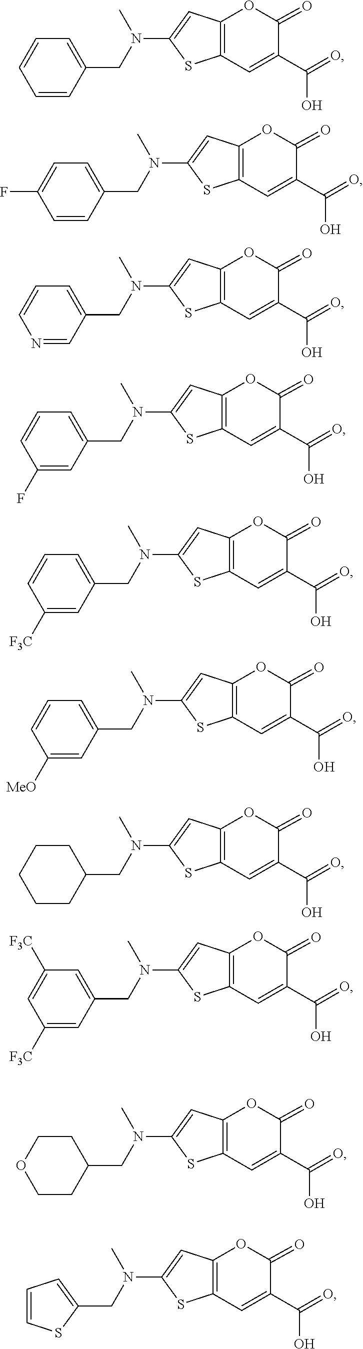

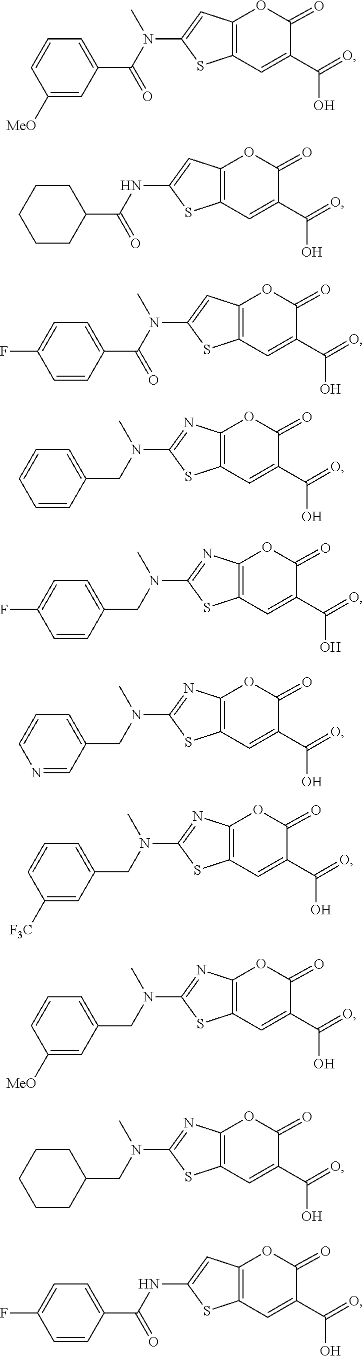

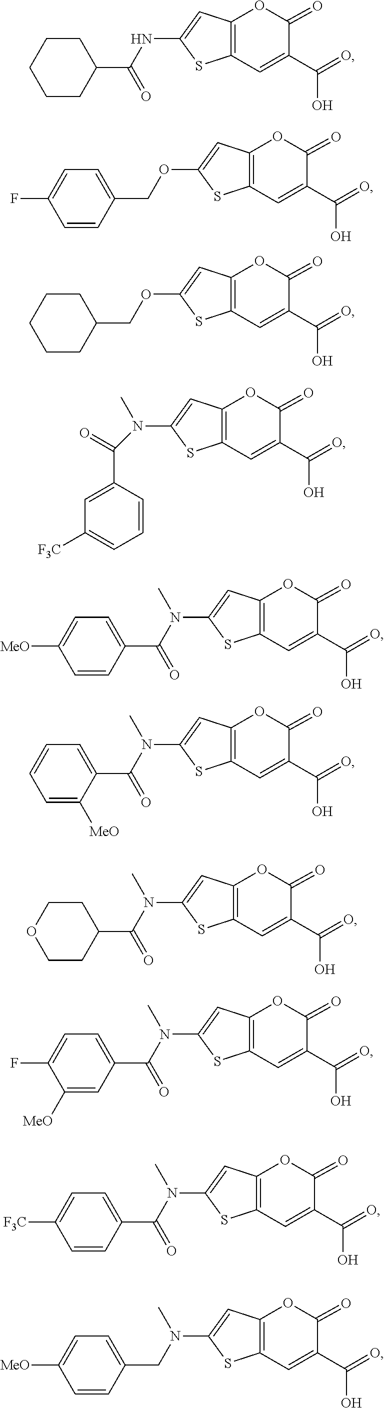

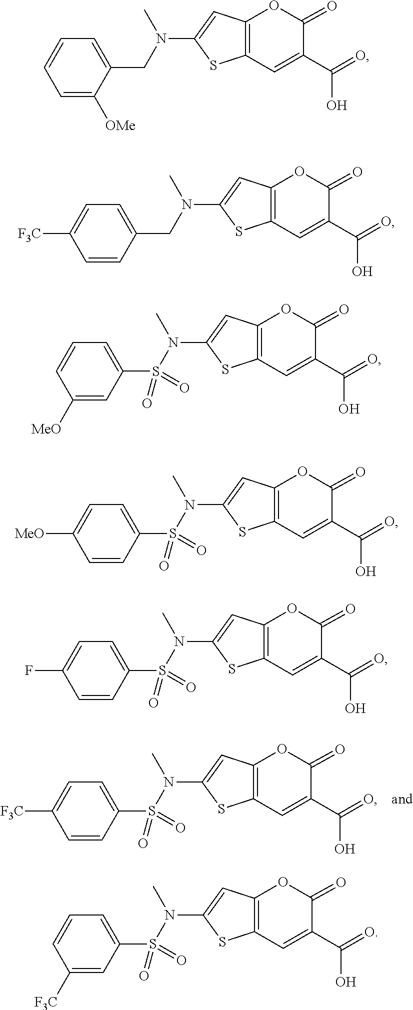

In some embodiments, the compound is selected from the group consisting of:

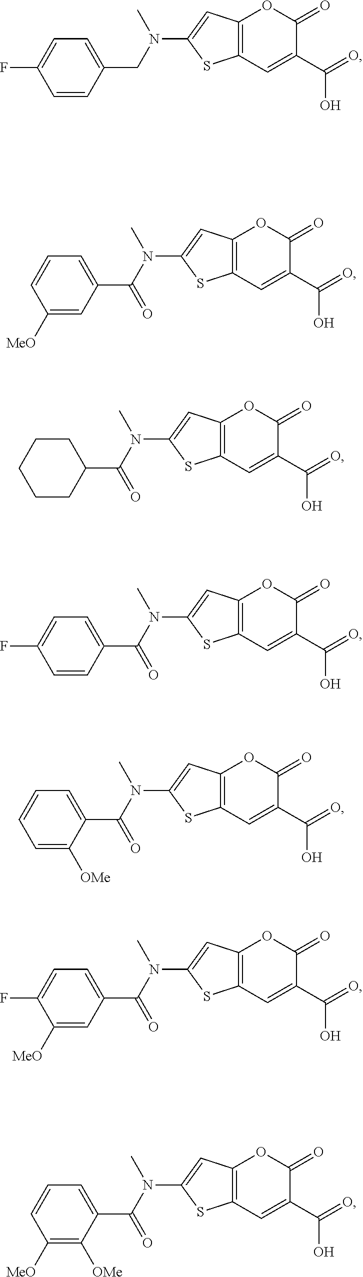

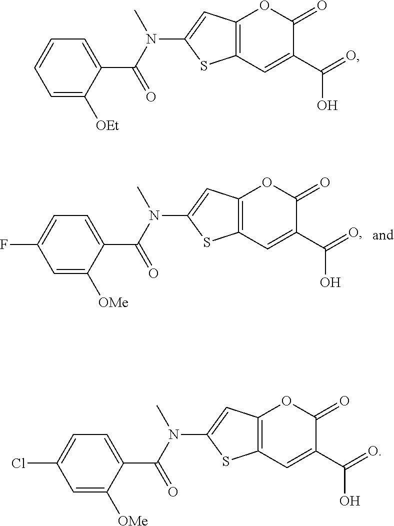 [SMILES: CCOc1ccccc1C(=O)N(C)c1cc2oc(=O)c(C(=O)O)cc2s1 ; COc1cc(F)ccc1C(=O)N(C)c1cc2oc(=O)c(C(=O)O)cc2s1 ; COc1cc(Cl)ccc1C(=O)N(C)c1cc2oc(=O)c(C(=O)O)cc2s1]

With reference to any one of formulae (I), (Ia) and (Ib), wherein W is O, NH, or NR″, in some embodiments, subscript n is 0; and Z is C═O. In some embodiments, the compound is represented by formula:

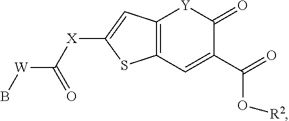

wherein B, X, Y, and R² are as defined and described herein; and W is O, NH, or NR″. In some embodiments, Y is O. In some embodiments, R² is hydrogen.

In some embodiments, the compound of formula (I) or (Ia) is represented by formula (III-2a):

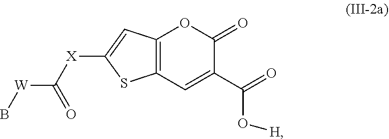

wherein B and X are as defined and described herein; and W is O, NH, or NR″.

With reference to any one of formulae (I), (Ia) and (Ib), wherein W is O, NH, or NR″, in some embodiments, Z is SO₂. In some embodiments, subscript n is 0 and Z is SO₂. In some embodiments, subscript n is 0, Z is SO₂, and Y is O. In some embodiments, subscript n is 0, Z is SO₂, Y is O, and R² is hydrogen. In some embodiments, the compound of formula (I) or (Ia) is represented by formula (IV):

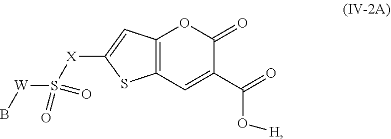

wherein B and X are as defined and described herein; and W is O, NH, or NR″.

With reference to any one of formulae (I), (Ia), (Ib), (III-2a), and (IV-2a), wherein W is O, NH, or NR″, in some embodiments, X is O. In some embodiments, X is NR″. In some embodiments, R″ is hydrogen. In some embodiments, R″ is C₁₋₆ alkyl. In some embodiments, R″ is methyl. In some embodiments, X is O, NH, or NMe. In some embodiments, X is NH or NMe. In some embodiments, X is NMe.

With reference to any one of formulae (I), (Ia), (Ib), (III-2a), and (IV-2a), in some embodiments, W is NH or NR″. In some embodiments, R″ is C₁₋₆ alkyl. In some embodiments, R″ is methyl. In some embodiments, W is NH or NMe. In some embodiments, W is NH. In some embodiments, W is NMe.

With reference to any one of formulae (I), (Ia), (Ib), and (III-2a), the compound is represented by formula (III-2a-1) or (III-2a-2):

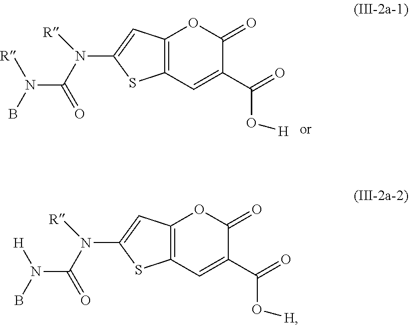

wherein B and each R″ are as defined and described herein.

In some embodiments of formula (III-2a-1), one R″ is hydrogen and the other R″ is C₁₋₆ alkyl. In some embodiments, each R″ is independently C₁₋₆ alkyl. In some embodiments of formula (III-2a-1), one R″ is hydrogen and the other R″ is methyl. In some embodiments, each R″ is methyl.

In some embodiments of formula (III-2a-2), R″ is C₁₋₆ alkyl. In some embodiments of formula (III-2a-2), R″ is methyl.

In some embodiments, the compound of formula (III-2a-1) or (III-2a-2) is selected from the group consisting of:

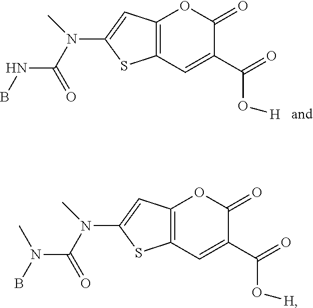

wherein B is as defined and described herein.

In some embodiments, W is O, NH, or NR″; and B ring is phenyl, optionally substituted with one or more substituents selected from R¹, R′ and R″, wherein R¹, R′ and R″ are as defined and described herein. In some embodiments, B ring is phenyl, optionally substituted with one or more substituents selected from the group consisting of halogen, OH, CN, C₁₋₆ alkyl, C₁₋₆ haloalkyl, C₃₋₈ cycloalkyl, C₁₋₆ alkoxy, C₁₋₆ haloalkoxy, and O-phenyl optionally substituted with halogen. In some embodiments, B is phenyl, optionally substituted with one or more substituents selected from the group consisting of halogen, OH, CN, C₁₋₆ alkyl, C₁₋₆ haloalkyl, C₁₋₆ alkoxy, and C₁₋₆ haloalkoxy. In some embodiments, B is phenyl, optionally substituted with one or more substituents selected from the group consisting of F, Cl, OH, CN, methyl, ethyl, isopropyl, tert-butyl, cyclopropyl, CF₃, OMe, OEt, OCF₃, and C(O)Me. In some embodiments, B is phenyl, optionally substituted with one or more substituents selected from the group consisting of F, Cl, OH, CN, methyl, CF₃, OMe, OEt, and OCF₃.

In some embodiments, W is O, NH, or NR″; and B ring is selected from the group consisting of:

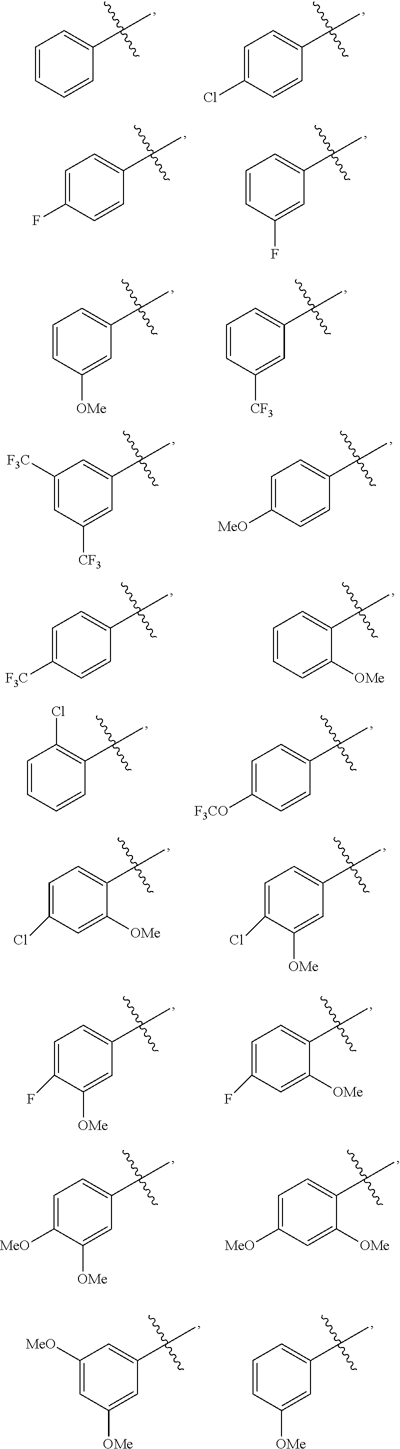

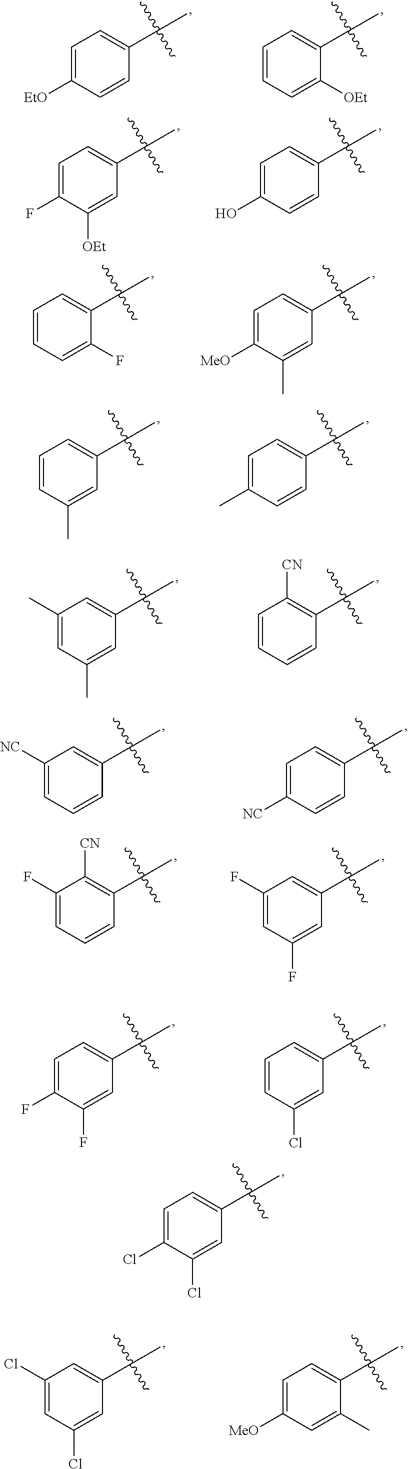

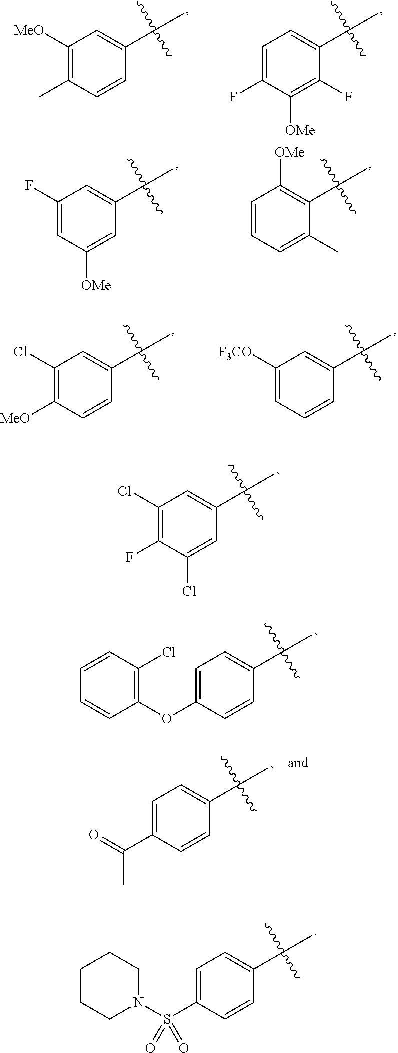

In some embodiments, W is O, NH, or NR″; and B ring is a 5-6 membered monocyclic heteroaryl ring having 1-4 heteroatoms independently selected from nitrogen, oxygen, and sulfur, optionally substituted with one or more substituents selected from R′, R′ and R″, wherein R′, R′ and R″ are as defined and described herein. In some embodiments, B ring is a 5-6 membered monocyclic heteroaryl ring having 1-4 heteroatoms independently selected from nitrogen, oxygen, and sulfur, optionally substituted with one or more substituents selected from the group consisting of halogen, OH, CN, C₁₋₆ alkyl, C₁₋₆ haloalkyl, C₃₋₈ cycloalkyl, C₁₋₆ alkoxy, C₁₋₆ haloalkoxy, and phenyl optionally substituted with C₁₋₆ alkoxy. In some embodiments, B is a 5-6 membered monocyclic heteroaryl ring having 1-4 heteroatoms independently selected from nitrogen, oxygen, and sulfur, optionally substituted with one or more substituents selected from the group consisting of halogen, OH, CN, C₁₋₆ alkyl, C₁₋₆ haloalkyl, C₁₋₆ alkoxy, and C₁₋₆ haloalkoxy. In some embodiments, B is a 5-6 membered monocyclic heteroaryl ring having 1-4 heteroatoms independently selected from nitrogen, oxygen, and sulfur, optionally substituted with one or more substituents selected from the group consisting of F, Cl, OH, CN, methyl, ethyl, isopropyl, tert-butyl, cyclopropyl, CF₃, OMe, OEt, OCF₃, C(O)Me. In some embodiments, B is a 5-6 membered monocyclic heteroaryl ring having 1-4 heteroatoms independently selected from nitrogen, oxygen, and sulfur, optionally substituted with one or more substituents selected from the group consisting of F, Cl, OH, CN, methyl, CF₃, OMe, OEt, and OCF₃.

In some embodiments, W is O, NH, or NR″; and B ring is selected from the group consisting of:

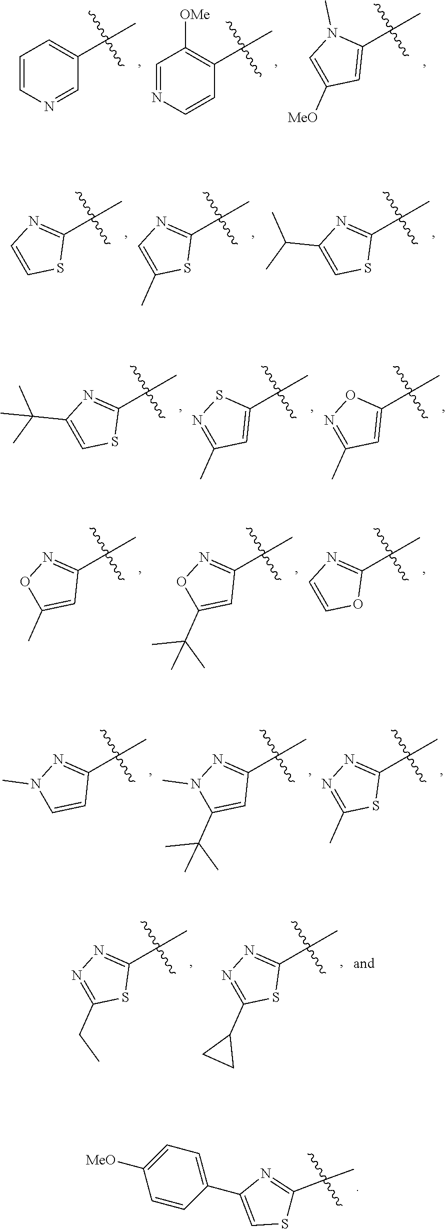

In some embodiments, W is O, NH, or NR″; and B ring is a 8-10 membered bicyclic heteroaryl ring having 1-4 heteroatoms independently selected from nitrogen, oxygen, and sulfur, optionally substituted with one or more substituents selected from R′, R′ and R″, wherein R′, R′ and R″ are as defined and described herein. In some embodiments, B ring is a 8-10 membered bicyclic heteroaryl ring having 1-4 heteroatoms independently selected from nitrogen, oxygen, and sulfur, optionally substituted with one or more substituents selected from the group consisting of halogen, OH, CN, C₁₋₆ alkyl, C₁₋₆ haloalkyl, C₃₋₈ cycloalkyl, C₁₋₆ alkoxy, and C₁₋₆ haloalkoxy. In some embodiments, B is a 8-10 membered bicyclic heteroaryl ring having 1-4 heteroatoms independently selected from nitrogen, oxygen, and sulfur, optionally substituted with one or more substituents selected from the group consisting of halogen, OH, CN, C₁₋₆ alkyl, C₁₋₆ haloalkyl, C₁₋₆ alkoxy, and C₁₋₆ haloalkoxy. In some embodiments, B is a 8-10 membered bicyclic heteroaryl ring having 1-4 heteroatoms independently selected from nitrogen, oxygen, and sulfur, optionally substituted with one or more substituents selected from the group consisting of F, Cl, OH, CN, methyl, ethyl, isopropyl, tert-butyl, cyclopropyl, CF₃, OMe, OEt, OCF₃, and C(O)Me. In some embodiments, B is a 8-10 membered bicyclic heteroaryl ring having 1-4 heteroatoms independently selected from nitrogen, oxygen, and sulfur, optionally substituted with one or more substituents selected from the group consisting of F, Cl, OH, CN, methyl, CF₃, OMe, OEt, and OCF₃.

In some embodiments, W is O, NH, or NR″; and B ring is selected from the group consisting of:

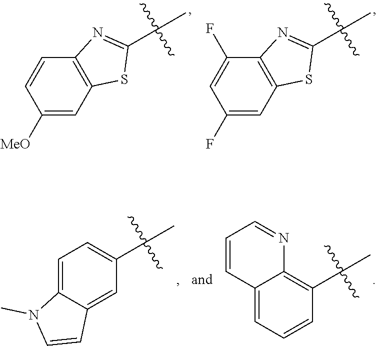 [SMILES: *c1nc2ccc(OC)cc2s1 ; *c1nc2c(F)cc(F)cc2s1 ; *c1ccc2c(ccn2C)c1 ; *c1cccc2cccnc12]

In some embodiments, W is O, NH, or NR″; B ring is cyclopentyl or cyclohexyl, optionally substituted with one or more substituents selected from R′, R′ and R″, wherein R′, R′ and R″ are as defined and described herein. In some embodiments, B ring is cyclopentyl or cyclohexyl, optionally substituted with one or more substituents selected from the group consisting of halogen, OH, CN, C₁₋₆ alkyl, C₁₋₆ haloalkyl, C₃₋₈ cycloalkyl, C₁₋₆ alkoxy, and C₁₋₆ haloalkoxy. In some embodiments, B is cyclopentyl or cyclohexyl, optionally substituted with one or more substituents selected from the group consisting of halogen, OH, CN, C₁₋₆ alkyl, C₁₋₆ haloalkyl, C₁₋₆ alkoxy, and C₁₋₆ haloalkoxy. In some embodiments, B is cyclopentyl or cyclohexyl, optionally substituted with one or more substituents selected from the group consisting of F, Cl, OH, CN, methyl, ethyl, isopropyl, tert-butyl, cyclopropyl, CF₃, OMe, OEt, OCF₃, and C(O)Me. In some embodiments, B is cyclopentyl or cyclohexyl, optionally substituted with one or more substituents selected from the group consisting of F, Cl, OH, CN, methyl, CF₃, OMe, OEt, and OCF₃.

In some embodiments, W is O, NH, or NR″; and B ring is selected from the group consisting of:

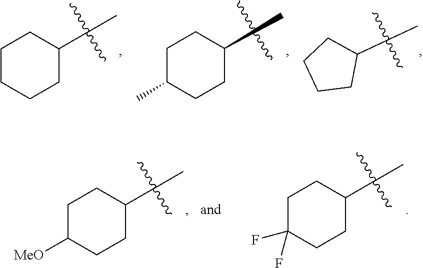

In some embodiments, W is O, NH, or NR″; B ring is a 5-6 membered saturated monocyclic heterocyclic ring having 1-2 heteroatoms independently selected from nitrogen, oxygen, and sulfur, optionally substituted with one or more substituents selected from R′, R′ and R″, wherein R¹, R′ and R″ are as defined and described herein. In some embodiments, B ring is a 5-6 membered saturated monocyclic heterocyclic ring having 1-2 heteroatoms independently selected from nitrogen, oxygen, and sulfur, optionally substituted with one or more substituents selected from the group consisting of halogen, OH, CN, C₁₋₆ alkyl, C₁₋₆ haloalkyl, C₃₋₈ cycloalkyl, C₁₋₆ alkoxy, and C₁₋₆ haloalkoxy. In some embodiments, B is a 5-6 membered saturated monocyclic heterocyclic ring having 1-2 heteroatoms independently selected from nitrogen, oxygen, and sulfur, optionally substituted with one or more substituents selected from the group consisting of halogen, OH, CN, C₁₋₆ alkyl, C₁₋₆ haloalkyl, C₁₋₆ alkoxy, and C₁₋₆ haloalkoxy. In some embodiments, B is a 5-6 membered saturated monocyclic heterocyclic ring having 1-2 heteroatoms independently selected from nitrogen, oxygen, and sulfur, optionally substituted with one or more substituents selected from the group consisting of F, Cl, OH, CN, methyl, ethyl, isopropyl, tert-butyl, cyclopropyl, CF₃, OMe, OEt, OCF₃, and C(O)Me. In some embodiments, B is a 5-6 membered saturated monocyclic heterocyclic ring having 1-2 heteroatoms independently selected from nitrogen, oxygen, and sulfur, optionally substituted with one or more substituents selected from the group consisting of F, Cl, OH, CN, methyl, CF₃, OMe, OEt, and OCF₃.

In some embodiments, W is O, NH, or NR″; B ring is selected from the group consisting of:

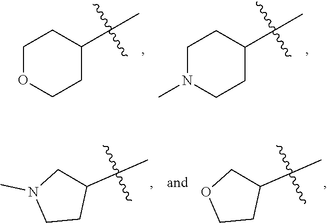

In some embodiments, the compound is selected from the group consisting of:

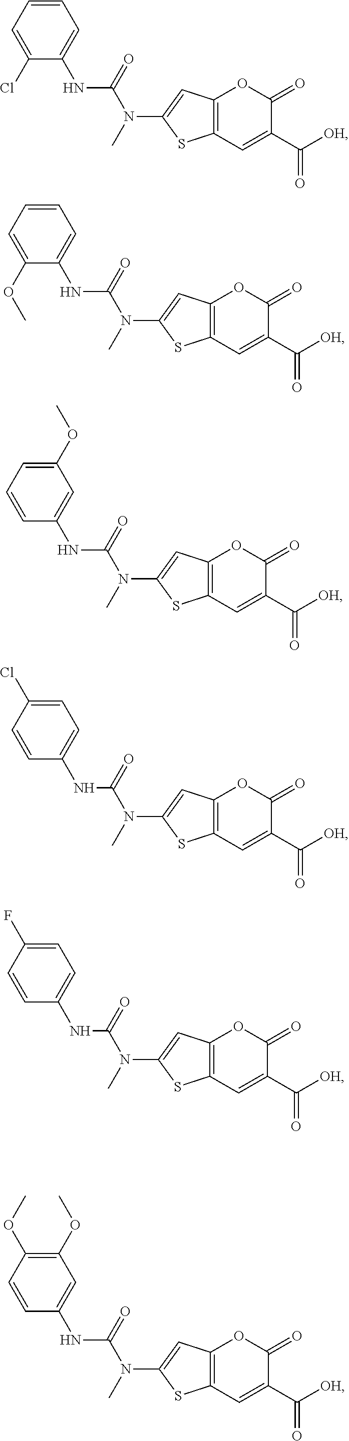 [SMILES: CN(C(=O)Nc1ccccc1Cl)c1cc2oc(=O)c(C(=O)O)cc2s1 ; COc1ccccc1NC(=O)N(C)c1cc2oc(=O)c(C(=O)O)cc2s1 ; COc1cccc(NC(=O)N(C)c2cc3oc(=O)c(C(=O)O)cc3s2)c1 ; CN(C(=O)Nc1ccc(Cl)cc1)c1cc2oc(=O)c(C(=O)O)cc2s1 ; CN(C(=O)Nc1ccc(F)cc1)c1cc2oc(=O)c(C(=O)O)cc2s1 ; COc1ccc(NC(=O)N(C)c2cc3oc(=O)c(C(=O)O)cc3s2)cc1OC]

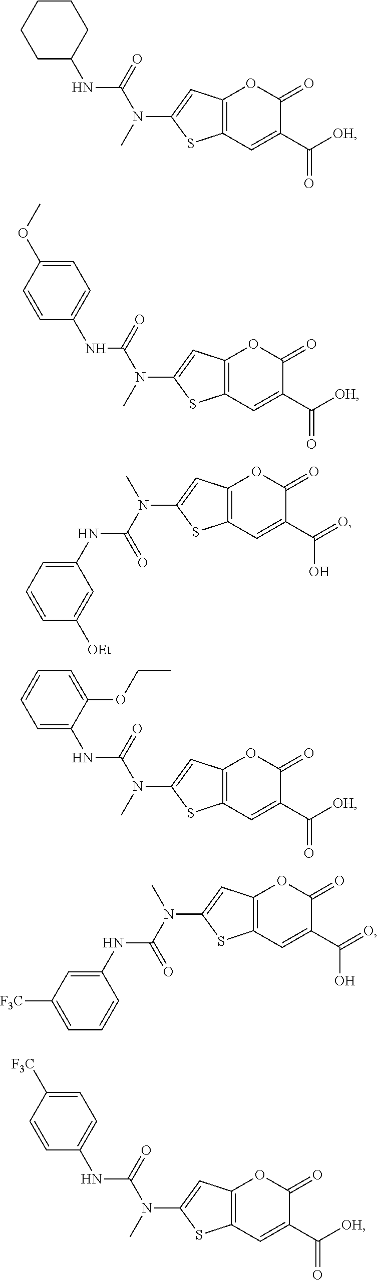

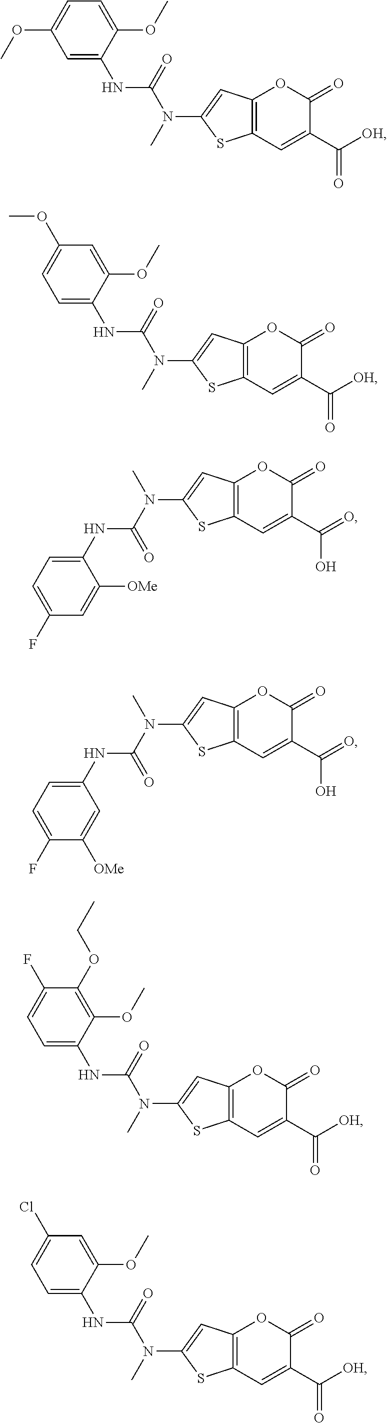

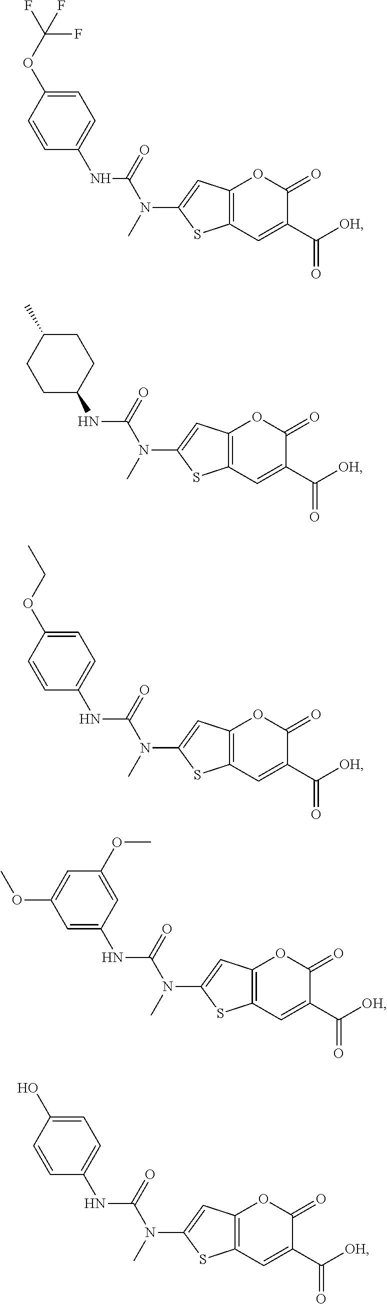 [SMILES: CN(C(=O)Nc1ccc(OC(F)(F)F)cc1)c1cc2oc(=O)c(C(=O)O)cc2s1 ; CN(C(=O)N[C@H]1CC[C@H](C)CC1)c1cc2oc(=O)c(C(=O)O)cc2s1 ; CCOc1ccc(NC(=O)N(C)c2cc3oc(=O)c(C(=O)O)cc3s2)cc1 ; COc1cc(NC(=O)N(C)c2cc3oc(=O)c(C(=O)O)cc3s2)cc(OC)c1 ; CN(C(=O)Nc1ccc(O)cc1)c1cc2oc(=O)c(C(=O)O)cc2s1]

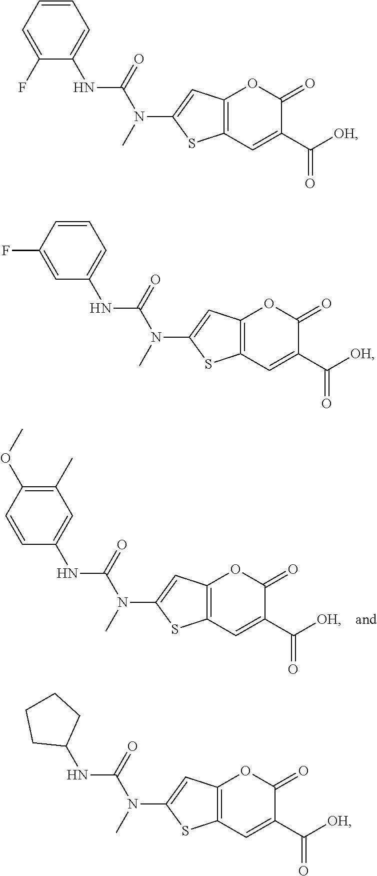 [SMILES: CN(C(=O)Nc1ccccc1F)c1cc2oc(=O)c(C(=O)O)cc2s1 ; CN(C(=O)Nc1cccc(F)c1)c1cc2oc(=O)c(C(=O)O)cc2s1 ; COc1ccc(NC(=O)N(C)c2cc3oc(=O)c(C(=O)O)cc3s2)cc1C ; "and" ; CN(C(=O)NC1CCCC1)c1cc2oc(=O)c(C(=O)O)cc2s1]

or a pharmaceutically acceptable salt thereof.

In some embodiments, the compound is selected from the group consisting of:

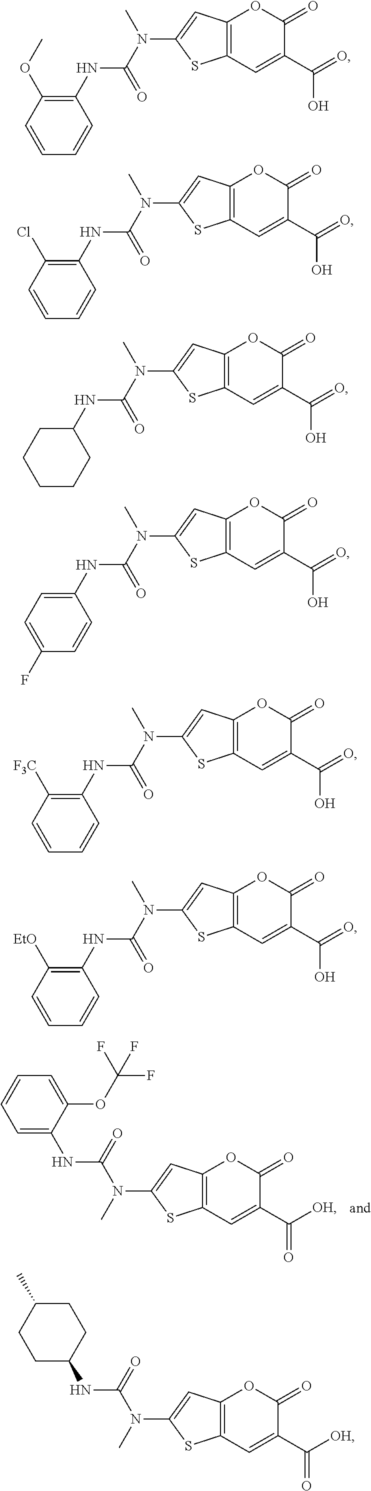

or a pharmaceutically acceptable salt thereof.

In some embodiments, the compound is selected from the group consisting of:

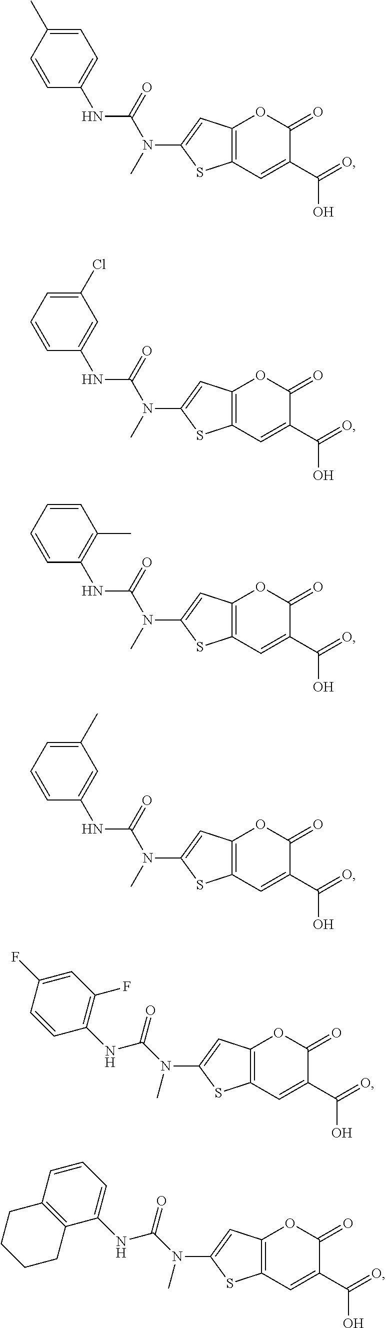 [SMILES: Cc1ccc(NC(=O)N(C)c2cc3oc(=O)c(C(=O)O)cc3s2)cc1 ; CN(C(=O)Nc1cccc(Cl)c1)c1cc2oc(=O)c(C(=O)O)cc2s1 ; Cc1ccccc1NC(=O)N(C)c1cc2oc(=O)c(C(=O)O)cc2s1 ; Cc1cccc(NC(=O)N(C)c2cc3oc(=O)c(C(=O)O)cc3s2)c1 ; CN(C(=O)Nc1ccc(F)cc1F)c1cc2oc(=O)c(C(=O)O)cc2s1 ; CN(C(=O)Nc1cccc2c1CCCC2)c1cc2oc(=O)c(C(=O)O)cc2s1]

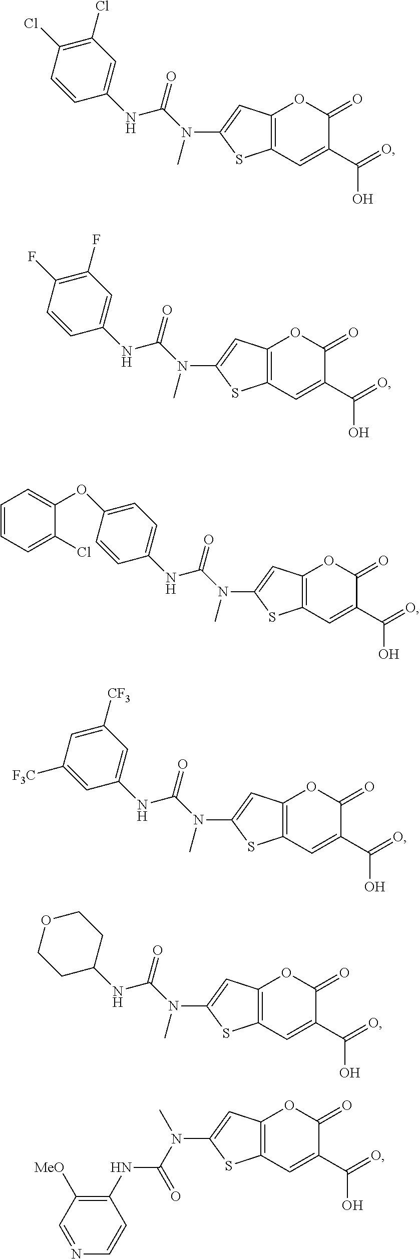

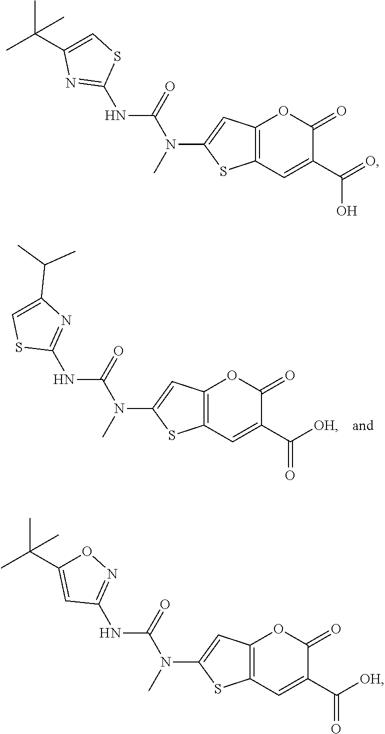 [SMILES: CN(C(=O)Nc1nc(C(C)(C)C)cs1)c1cc2oc(=O)c(C(=O)O)cc2s1 ; CC(C)c1csc(NC(=O)N(C)c2cc3oc(=O)c(C(=O)O)cc3s2)n1 ; "and" ; CN(C(=O)Nc1cc(C(C)(C)C)on1)c1cc2oc(=O)c(C(=O)O)cc2s1]

or a pharmaceutically acceptable salt thereof.

In some embodiments, the compound is selected from the group consisting of:

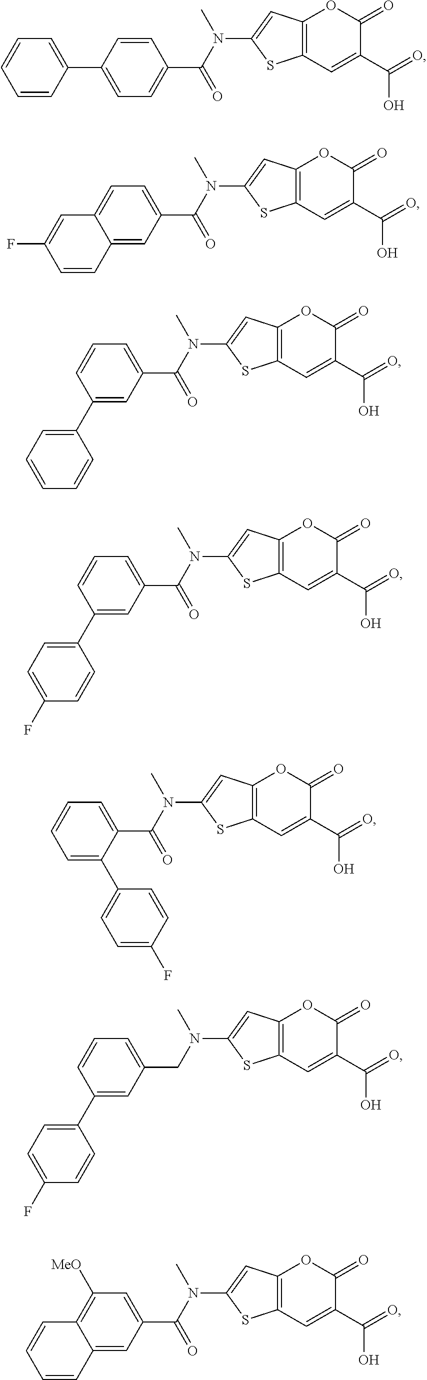

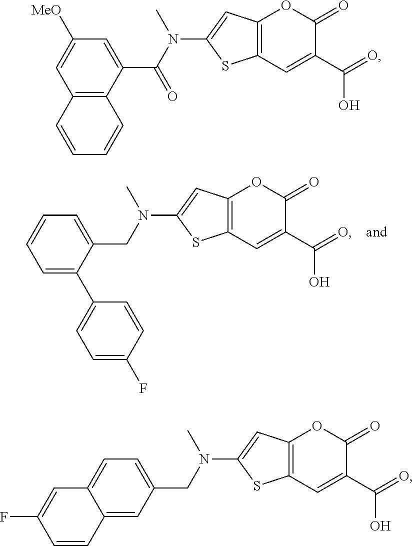

or a pharmaceutically acceptable salt thereof.

In some embodiments, the compound is selected from the group consisting of:

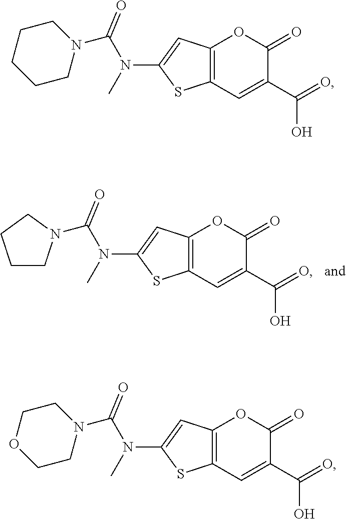 [SMILES: CN(C(=O)N1CCCCC1)c1cc2oc(=O)c(C(=O)O)cc2s1 ; CN(C(=O)N1CCCC1)c1cc2oc(=O)c(C(=O)O)cc2s1 ; "and" ; CN(C(=O)N1CCOCC1)c1cc2oc(=O)c(C(=O)O)cc2s1]

or a pharmaceutically acceptable salt thereof.

In some embodiments, the compound is selected from the group consisting of:

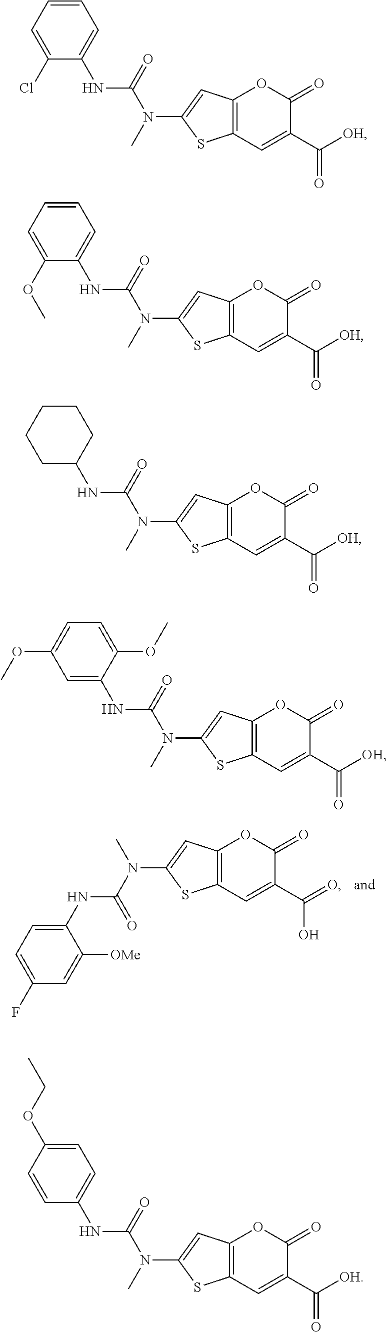

Exemplified compounds of formula (I) or bicyclic enone carboxylic acid compounds are listed in Tables 1 and 2.

In some embodiments, the present invention provides a compound of formula (I) or a bicyclic enone carboxylic acid compound according to Tables 1 and 2.

As used herein, and as would be understood by the person of skill in the art, the recitation of “a compound”—unless expressly further limited—is intended to include salts of that compound. Thus, for example, the recitation “a compound of formula (I)” as depicted above, in which R² is H, would include salts in which the carboxylic acid is of the formula COO⁻ M⁺, wherein M is any counterion. In a particular embodiment, the term “compound of formula (I)” refers to the compound or a pharmaceutically acceptable salt thereof. Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

In some embodiments, the base addition salt is formed from sodium, potassium, magnesium, or calcium.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

### V. Composition

In a second aspect, the present invention provides a pharmaceutical composition for treating cancer in a subject, the composition including:
a) a therapeutically effective amount of a monocarboxylate transporter (MCT) inhibitor; and
b) a therapeutically effective amount of an immune therapy agent, together with a pharmaceutically acceptable carrier or excipient, wherein the MCT inhibitor is represented by Formula (I) as defined and described herein.

The cancer and the subject are each described according to Section III: Method. In some embodiments, the cancer is any of embodiments as described in Section III: Method. In some embodiments, the subject is any of embodiments as described in Section III: Method.

The MCT inhibitor of Formula (I) is further described according to Section IV. Compounds. In some embodiments, the MCT inhibitor of Formula (I) is any of embodiments as described in Section IV. Compounds.

In some embodiments, the immune therapy agent is as described in Section III: Method. In some embodiments, the immunotherapy agent is an immune checkpoint inhibitor, a chimeric antigen receptor (CAR) therapy agent, a vaccine, a modulator of myeloid cells or a macrophage, a modulator of one or more cytokines, or combinations thereof. In some embodiments, the immunotherapy agent is an immune checkpoint inhibitor. In some embodiments, the immune checkpoint inhibitor is a PD-1/PD-L1 inhibitor. In some embodiments, the PD-1/PD-L1 inhibitor is pembrolizumab, nivolumab, atezolizumab, durvalumab, avelumab, cemiplimab-rwlc, camrelizumab, JS001, sintilimab, prolgolimab, tislelizumab, balstilimab, dostarlimab, or retifanlimab. In some embodiments, the immune checkpoint inhibitor is a CTLA-4 inhibitor. In some embodiments, the CTLA-4 inhibitor is ipilimumab or tremelimumab. In some embodiments, the CTLA-4 inhibitor is ipilimumab.

The compositions of the present disclosure can be prepared in a wide variety of oral, parenteral and topical dosage forms. Oral preparations include tablets, pills, powder, dragees, capsules, liquids, lozenges, cachets, gels, syrups, slurries, suspensions, etc., suitable for ingestion by the patient. The compositions of the present disclosure can also be administered by injection, that is, intravenously, intramuscularly, intracutaneously, subcutaneously, intraduodenally, or intraperitoneally. Also, the compositions described herein can be administered by inhalation, for example, intranasally. Additionally, the compositions of the present disclosure can be administered transdermally. The compositions of this disclosure can also be administered by intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995).

For preparing pharmaceutical compositions of the present disclosure, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton Pa. (“Remington's”).

In powders, the carrier is a finely divided solid, which is in a mixture with the finely divided active component. In tablets, the active components are mixed with the carrier having the necessary binding properties in suitable proportions and compacted in a particular shape and size.

The powders, capsules and tablets preferably contain from about 5% to about 70% of the active compound, such as from about 10% to about 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other excipients, is surrounded by a carrier, which is thus in association with it. Similarly, cachets and lozenges are included. Tablets, powders, capsules, pills, cachets, and lozenges can be used as solid dosage forms suitable for oral administration.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins including, but not limited to, gelatin and collagen. Disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the present disclosure can also be used orally using, for example, push-fit capsules made of gelatin, as well as soft, sealed capsules made of gelatin and a coating such as glycerol or sorbitol. Push-fit capsules can contain the compound of Formula (I) mixed with a filler or binders such as lactose or starches, lubricants such as talc or magnesium stearate, and, optionally, stabilizers. In soft capsules, the compound of Formula (I) may be dissolved or suspended in suitable liquids, such as fatty oils, liquid paraffin, or liquid polyethylene glycol with or without stabilizers.

For preparing suppositories, a low melting wax, such as a mixture of fatty acid glycerides or cocoa butter, is first melted and the compound of Formula (I) are dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the compound of Formula (I) and the immunotherapy agent, as defined and described herein, in water and adding optional suitable colorants, flavors, stabilizers, and thickening agents. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending the compound of Formula (I) and the immunotherapy agent, as defined and described herein, in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the present disclosure can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The compositions of the present disclosure can be delivered by any suitable means, including oral, parenteral and topical methods. Transdermal administration methods, by a topical route, can be formulated as applicator sticks, solutions, suspensions, emulsions, gels, creams, ointments, pastes, jellies, paints, powders, and aerosols.

The compositions of the present disclosure can also be delivered as microspheres for slow release in the body. For example, microspheres can be formulated for administration via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

In another embodiment, the compositions of the present disclosure can be formulated for parenteral administration, such as intravenous (IV) administration or administration into a body cavity or lumen of an organ. The formulations for administration will commonly comprise a solution of the compositions of the present disclosure dissolved in a pharmaceutically acceptable carrier. Among the acceptable vehicles and solvents that can be employed are water and Ringer's solution, an isotonic sodium chloride. In addition, sterile fixed oils can be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by various sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the compositions of the present disclosure in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In another embodiment, the formulations of the compositions of the present disclosure can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the compositions of the present disclosure into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

Lipid-based drug delivery systems include lipid solutions, lipid emulsions, lipid dispersions, self-emulsifying drug delivery systems (SEDDS) and self-microemulsifying drug delivery systems (SMEDDS). In particular, SEDDS and SMEDDS are isotropic mixtures of lipids, surfactants and co-surfactants that can disperse spontaneously in aqueous media and form fine emulsions (SEDDS) or microemulsions (SMEDDS). Lipids useful in the formulations of the present disclosure include any natural or synthetic lipids including, but not limited to, sesame seed oil, olive oil, castor oil, peanut oil, fatty acid esters, glycerol esters, Labrafil®, Labrasol®, Cremophor®, Solutol®, Tween®, Capryol®, Capmul®, Captex®, and Peceol®.

The pharmaceutical formulations of the present disclosure can be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preparation may be a lyophilized powder in, e.g., 1 mM-50 mM histidine, 0.1%-2% sucrose, 2%-7% mannitol at a pH range of 4.5 to 5.5, that is combined with buffer prior to use.

The pharmaceutical formulations of the present disclosure can be provided as a salt and can be formed with bases, namely cationic salts such as alkali and alkaline earth metal salts, such as sodium, lithium, potassium, calcium, magnesium, as well as ammonium salts, such as ammonium, trimethyl-ammonium, diethylammonium, and tris-(hydroxymethyl)-methyl-ammonium salts.

### VI. Kits

In a third aspect, the present invention provides a kit for treating cancer in a subject, the kit including:
a) a therapeutically effective amount of a monocarboxylate transporter (MCT) inhibitor; and
b) a therapeutically effective amount of an immune therapy agent, with a pharmaceutically acceptable carrier or excipient,
   wherein the MCT inhibitor is represented by Formula (I) as defined and described herein.

The cancer and the subject are each described according to Section III: Method. In some embodiments, the cancer is any of embodiments as described in Section III: Method. In some embodiments, the subject is any of embodiments as described in Section III: Method.

The MCT inhibitor of Formula (I) is further described according to Section IV. Compounds. In some embodiments, the MCT inhibitor of Formula (I) is any of embodiments as described in Section IV. Compounds.

In some embodiments, the immune therapy agent is as described in Section III: Method. In some embodiments, the immunotherapy agent is an immune checkpoint inhibitor, a chimeric antigen receptor (CAR) therapy agent, a vaccine, a modulator of myeloid cells or a macrophage, a modulator of one or more cytokines, or combinations thereof. In some embodiments, the immunotherapy agent is an immune checkpoint inhibitor. In some embodiments, the immune checkpoint inhibitor is a PD-1/PD-L1 inhibitor. In some embodiments, the PD-1/PD-L1 inhibitor is pembrolizumab, nivolumab, atezolizumab, durvalumab, avelumab, cemiplimab-rwlc, camrelizumab, JS001, sintilimab, prolgolimab, tislelizumab, balstilimab, dostarlimab, or retifanlimab. In some embodiments, the immune checkpoint inhibitor is a CTLA-4 inhibitor. In some embodiments, the CTLA-4 inhibitor is ipilimumab or tremelimumab. In some embodiments, the CTLA-4 inhibitor is ipilimumab.

The MCT inhibitor as described herein and the immunotherapy agent as described herein can be are formulated for concomitant administration or sequential administration. In some embodiments, the MCT inhibitor and the immune therapy agent are formulated for concomitant administration. In some embodiments, the MCT inhibitor and the immune therapy agent are formulated for sequential administration.

### VII. Examples

### Abbreviations:

### atm Atmosphere

aq. Aqueous
h hour(s)
HPLC High performance liquid chromatography
LCMS HPLC mass spec

### min Minutes

o.n. Over night
RT, rt, r.t. Room temperature
nM nanomolar

### TLC Thin Layer Chromatography

SPE Solid phase extraction (usually containing silica gel for mini-chromatography)
sat. Saturated
uM micromolar
mins minutes

### Example 1: MTS Cell Proliferation Assay

Cytotoxicity of the inhibition of monocarboxylate transporters of the invention was determined and shown in Table 1. The anti-proliferation effect of MCT inhibition was investigated across a panel of solid and haemotological tumor cell lines. Cells were routinely cultured in their appropriate growth medium. On day 1, between 10,000-25,000 cells/well (e.g., Hs578t: 15,000 cells/well, SiHa: 10,000 cells/well, and MDA-MB-231: 25,000 cells/well) were plated into 96-well plates. 100 μL. of phosphate buffered saline solution was added to the external wells to prevent media evaporation. Plates were incubated in growth medium overnight at 37° C. in the presence of 5% CO₂. On day 2, dry weight compound stocks were dissolved to a concentration of 10 mM in 100% DMSO. Compounds were further diluted in the assay medium; 10 mM lactate medium (without glucose, pyruvate, and glutamine) or RPMI 1640 medium (without pyruvate and glutamine) to generate a final dose range of 1 nM to 10 μM. Growth medium in the 96-well plate was replaced with the assay medium (10 mM lactate medium or RPMI medium or appropriate medium), and compounds were added to each well in the plate at different concentrations via serial dilution or pre-prepared solutions in assay medium. Plates were then incubated at 37° C. in the presence of 5% CO₂ for a further 72-96 hours. On day 2-5, assay media was changed to 100 uL of DMEM/F12 and 20 μL of CellTiter 96 AQ MTS reagent was added to each well and the plate was returned to the incubator for 1-2 hours. MTS is bioreduced by NADPH or NADH produced by dehydrogenase enzymes in metabolically active cells into a coloured formazan product that is soluble in tissue culture medium. The amount of coloured formazan product is directly proportional to the number of living cells in culture. The absorbance of the plates was read on a Spectramax M5e plate reader using 490 nM measurement wavelength. Dose response curves were plotted and IC₅₀ values were calculated using GraphPad Prism. The IC₅₀ value is equivalent to the concentration of compound that causes 50% inhibition of growth calculated from the compound treated signal to the vehicle treated signal.

Cytotoxicity of selected compounds is listed in Table 1, where IC₅₀: A=<1 uM; B=1-10 uM; C=>10 uM; and NT=Not Tested.

TABLE 1
Compounds and Assay Results (Group 1)

| | | | Cytotoxicity |
|---|---|---|---|
| ID | Structure | Name | [IC₅₀: μM] |
| 1.001 | 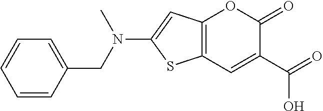 [SMILES: CN(Cc1ccccc1)c1cc2oc(=O)c(C(=O)O)cc2s1] | 2-(benzyl(methyl)amino)-5- oxo-5H-thieno[3,2-b]pyran-6- carboxylic acid | A |
| 1.002 | 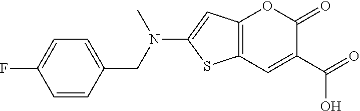 [SMILES: CN(Cc1ccc(F)cc1)c1cc2oc(=O)c(C(=O)O)cc2s1] | 2-((4- fluorobenzyl)(methyl)amino)- 5-oxo-5H-thieno[3,2-b]pyran- 6-carboxylic acid | A |
| 1.003 | 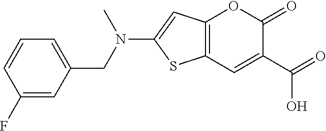 [SMILES: CN(Cc1cccc(F)c1)c1cc2oc(=O)c(C(=O)O)cc2s1] | 2-((3- fluorobenzyl)(methyl)amino)- 5-oxo-5H-thieno[3,2-b]pyran- 6-carboxylic acid | A |
| 1.004 | 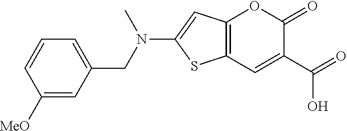 [SMILES: COc1cccc(CN(C)c2cc3oc(=O)c(C(=O)O)cc3s2)c1] | 2-((3- methoxybenzyl)(methyl)amino)- 5-oxo-5H-thieno[3,2- b]pyran-6-carboxylic acid | A |
| 1.005 | 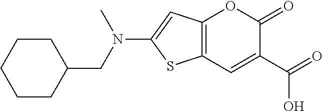 [SMILES: CN(CC1CCCCC1)c1cc2oc(=O)c(C(=O)O)cc2s1] | 2- ((cyclohexylmethyl)(methyl) amino)-5-oxo-5H-thieno[3,2- b]pyran-6-carboxylic acid | A |
| 1.006 | 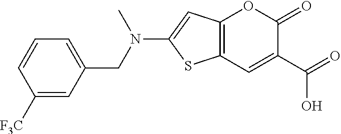 [SMILES: CN(Cc1cccc(C(F)(F)F)c1)c1cc2oc(=O)c(C(=O)O)cc2s1] | 2-(methyl(3- (trifluoromethyl)benzyl)amino)- 5-oxo-5H-thieno[3,2- b]pyran-6-carboxylic acid | A |
| 1.007 | 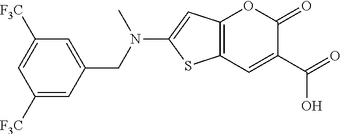 [SMILES: CN(Cc1cc(C(F)(F)F)cc(C(F)(F)F)c1)c1cc2oc(=O)c(C(=O)O)cc2s1] | 2-((3,5- bis(trifluoromethyl)benzyl) (methyl)amino)-5-oxo-5H- thieno[3,2-b]pyran-6- carboxylic acid | A |
| 1.008 | 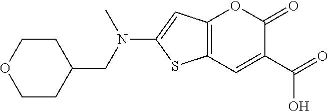 [SMILES: CN(CC1CCOCC1)c1cc2oc(=O)c(C(=O)O)cc2s1] | 2-(methyl((tetrahydro-2H- pyran-4-yl)methyl)amino)-5- oxo-5H-thieno[3,2-b]pyran-6- carboxylic acid | A |
| 1.009 | 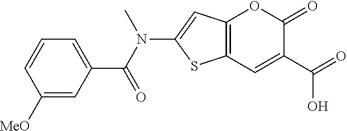 [SMILES: COc1cccc(C(=O)N(C)c2cc3oc(=O)c(C(=O)O)cc3s2)c1] | 2-(3-methoxy-N- methylbenzamido)-5-oxo-5H- thieno[3,2-b]pyran-6- carboxylic acid | A |
| 1.010 | 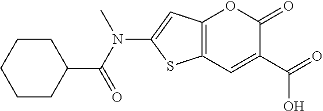 [SMILES: CN(C(=O)C1CCCCC1)c1cc2oc(=O)c(C(=O)O)cc2s1] | 2-(N- methylcyclohexanecarboxamido)- 5-oxo-5H-thieno[3,2- b]pyran-6-carboxylic acid | A |
| 1.011 | 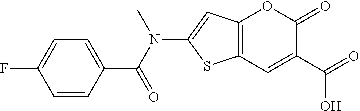 [SMILES: CN(C(=O)c1ccc(F)cc1)c1cc2oc(=O)c(C(=O)O)cc2s1] | 2-(4-fluoro-N- methylbenzamido)-5-oxo-5H- thieno[3,2-b]pyran-6- carboxylic acid | A |
| 1.012 | 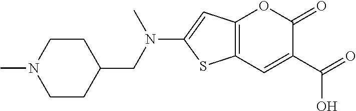 [SMILES: CN1CCC(CN(C)c2cc3oc(=O)c(C(=O)O)cc3s2)CC1] | 2-(methyl((1-methylpiperidin- 4-yl)methyl)amino)-5-oxo- 5H-thieno[3,2-b]pyran-6- carboxylic acid | A |
| 1.013 | 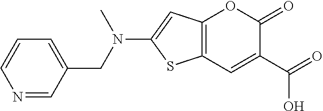 [SMILES: CN(Cc1cccnc1)c1cc2oc(=O)c(C(=O)O)cc2s1] | 2-(methyl(pyridin-3- ylmethyl)amino)-5-oxo-5H- thieno[3,2-b]pyran-6- carboxylic acid | A |
| 1.014 | 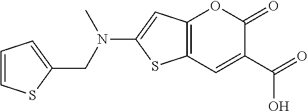 [SMILES: CN(Cc1cccs1)c1cc2oc(=O)c(C(=O)O)cc2s1] | 2-(methyl(thiophen-2- ylmethyl)amino)-5-oxo-5H- thieno[3,2-b]pyran-6- carboxylic acid | A |
| 1.015 | 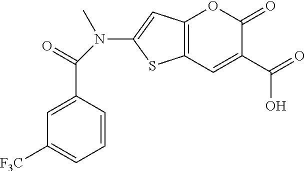 [SMILES: CN(C(=O)c1cccc(C(F)(F)F)c1)c1cc2oc(=O)c(C(=O)O)cc2s1] | 2-[Methyl-(3-trifluoromethyl- benzoyl)-amino]-5-oxo-5H- thieno[3,2-b]pyran-6- carboxylic acid | A |
| 1.016 | 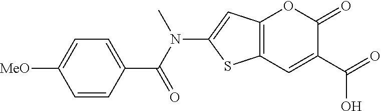 [SMILES: COc1ccc(C(=O)N(C)c2cc3oc(=O)c(C(=O)O)cc3s2)cc1] | 2-[(4-Methoxy-benzoyl)- methyl-amino]-5-oxo-5H- thieno[3,2-b]pyran-6- carboxylic acid | A |
| 1.017 | 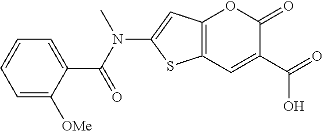 [SMILES: COc1ccccc1C(=O)N(C)c1cc2oc(=O)c(C(=O)O)cc2s1] | 2-[(2-Methoxy-benzoyl)- methyl-amino]-5-oxo-5H- thieno[3,2-b]pyran-6- carboxylic acid | A |
| 1.018 | 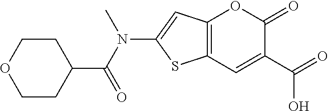 [SMILES: CN(C(=O)C1CCOCC1)c1cc2oc(=O)c(C(=O)O)cc2s1] | 2-[Methyl-(tetrahydro-pyran- 4-carbonyl)-amino]-5-oxo- 5H-thieno[3,2-b]pyran-6- carboxylic acid | A |
| 1.019 | 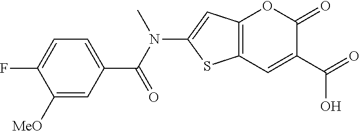 [SMILES: COc1cc(C(=O)N(C)c2cc3oc(=O)c(C(=O)O)cc3s2)ccc1F] | 2-[(4-Fluoro-3-methoxy- benzoyl)-methyl-amino]-5- oxo-5H-thieno[3,2-b]pyran-6- carboxylic acid | A |
| 1.020 | 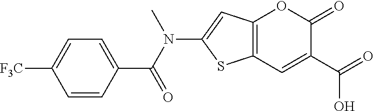 [SMILES: CN(C(=O)c1ccc(C(F)(F)F)cc1)c1cc2oc(=O)c(C(=O)O)cc2s1] | 2-[Methyl-(4-trifluoromethyl- benzoyl)-amino]-5-oxo-5H- thieno[3,2-b]pyran-6- carboxylic acid | A |
| 1.021 | 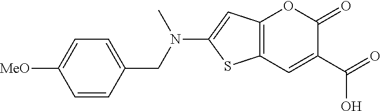 [SMILES: COc1ccc(CN(C)c2cc3oc(=O)c(C(=O)O)cc3s2)cc1] | 2-[(4-Methoxy-benzyl)- methyl-amino]-5-oxo-5H- thieno[3,2-b]pyran-6- carboxylic acid | A |
| 1.022 | 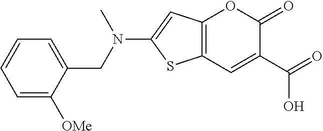 [SMILES: COc1ccccc1CN(C)c1cc2oc(=O)c(C(=O)O)cc2s1] | 2-[(2-Methoxy-benzyl)- methyl-amino]-5-oxo-5H- thieno[3,2-b]pyran-6- carboxylic acid | A |
| 1.023 | 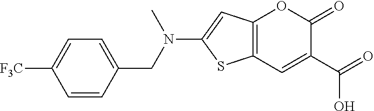 [SMILES: CN(Cc1ccc(C(F)(F)F)cc1)c1cc2oc(=O)c(C(=O)O)cc2s1] | 2-[Methyl-(4-trifluoromethyl- benzyl)-amino]-5-oxo-5H- thieno[3,2-b]pyran-6- carboxylic acid | A |
| 1.024 | 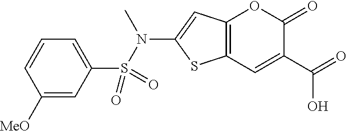 [SMILES: COc1cccc(S(=O)(=O)N(C)c2cc3oc(=O)c(C(=O)O)cc3s2)c1] | 2-[(3-Methoxy- benzenesulfonyl)-methyl- amino]-5-oxo-5H-thieno[3,2- b]pyran-6-carboxylic acid | A |
| 1.025 | 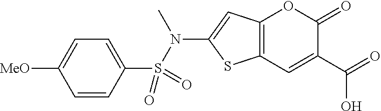 [SMILES: COc1ccc(S(=O)(=O)N(C)c2cc3oc(=O)c(C(=O)O)cc3s2)cc1] | 2-[(4-Methoxy- benzenesulfonyl)-methyl- amino]-5-oxo-5H-thieno[3,2- b]pyran-6-carboxylic acid | A |
| 1.026 | 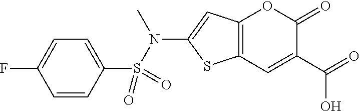 [SMILES: CN(c1cc2oc(=O)c(C(=O)O)cc2s1)S(=O)(=O)c1ccc(F)cc1] | 2-[(4-Fluoro- benzenesulfonyl)-methyl- amino]-5-oxo-5H-thieno[3,2- b]pyran-6-carboxylic acid | A |
| 1.027 | 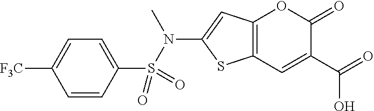 [SMILES: CN(c1cc2oc(=O)c(C(=O)O)cc2s1)S(=O)(=O)c1ccc(C(F)(F)F)cc1] | 2-[Methyl-(4-trifluoromethyl- benzenesulfonyl)-amino]-5- oxo-5H-thieno[3,2-b]pyran-6- carboxylic acid | A |
| 1.028 | 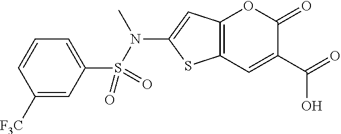 [SMILES: CN(c1cc2oc(=O)c(C(=O)O)cc2s1)S(=O)(=O)c1cccc(C(F)(F)F)c1] | 2-[Methyl-(3-trifluoromethyl- benzenesulfonyl)-amino]-5- oxo-5H-thieno[3,2-b]pyran-6- carboxylic acid | A |
| 1.029 | 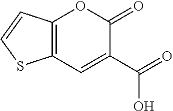 [SMILES: O=C(O)c1cc2sccc2oc1=O] | 5-oxo-5H-thieno[3,2-b]pyran- 6-carboxylic acid | B |
| 1.030 | 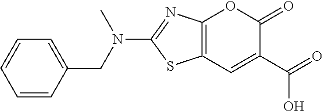 [SMILES: CN(Cc1ccccc1)c1nc2oc(=O)c(C(=O)O)cc2s1] | 2-(benzyl(methyl)amino)-5- oxo-5H-pyrano[2,3- d]thiazole-6-carboxylic acid | NT |
| 1.031 | 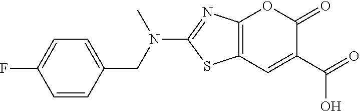 [SMILES: CN(Cc1ccc(F)cc1)c1nc2oc(=O)c(C(=O)O)cc2s1] | 2-((4- fluorobenzyl)(methyl)amino)- 5-oxo-5H-pyrano[2,3- d]thiazole-6-carboxylic acid | NT |
| 1.032 | 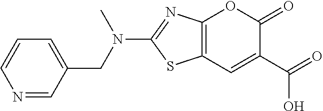 [SMILES: CN(Cc1cccnc1)c1nc2oc(=O)c(C(=O)O)cc2s1] | 2-(methyl(pyridin-3- ylmethyl)amino)-5-oxo-5H- pyrano[2,3-d]thiazole-6- carboxylic acid | NT |
| 1.033 | 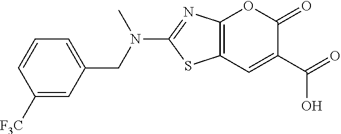 [SMILES: CN(Cc1cccc(C(F)(F)F)c1)c1nc2oc(=O)c(C(=O)O)cc2s1] | 2-(methyl(3- (trifluoromethyl)benzyl)amino)- 5-oxo-5H-pyrano[2,3- d]thiazole-6-carboxylic acid | NT |
| 1.034 | 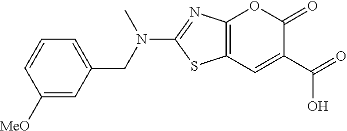 [SMILES: COc1cccc(CN(C)c2nc3oc(=O)c(C(=O)O)cc3s2)c1] | 2-((3- methoxybenzyl)(methyl)amino)- 5-oxo-5H-pyrano[2,3- d]thiazole-6-carboxylic acid | NT |
| 1.035 | 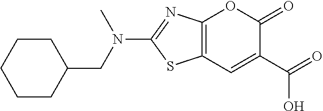 [SMILES: CN(CC1CCCCC1)c1nc2oc(=O)c(C(=O)O)cc2s1] | 2- ((cyclohexylmethyl)(methyl) amino)-5-oxo-5H-pyrano[2,3- d]thiazole-6-carboxylic acid | NT |
| 1.036 | 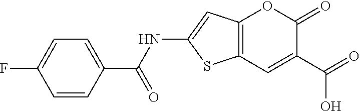 [SMILES: O=C(Nc1cc2oc(=O)c(C(=O)O)cc2s1)c1ccc(F)cc1] | 2-(4-fluorobenzamido)-5-oxo- 5H-thieno[3,2-b]pyran-6- carboxylic acid | NT |
| 1.037 | 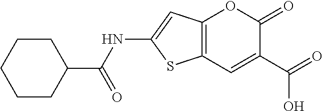 [SMILES: O=C(O)c1cc2sc(NC(=O)C3CCCCC3)cc2oc1=O] | 2-(cyclohexanecarboxamido)- 5-oxo-5H-thieno[3,2-b]pyran- 6-carboxylic acid | NT |
| 1.038 | 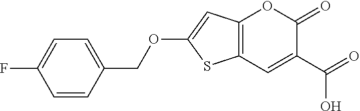 [SMILES: O=C(O)c1cc2sc(OCc3ccc(F)cc3)cc2oc1=O] | 2-((4-fluorobenzyl)oxy)-5- oxo-5H-thieno[3,2-b]pyran-6- carboxylic acid | NT |
| 1.039 | 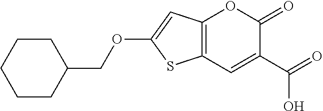 [SMILES: O=C(O)c1cc2sc(OCC3CCCCC3)cc2oc1=O] | 2-(cyclohexylmethoxy)-5- oxo-5H-thieno[3,2-b]pyran-6- carboxylic acid | NT |
| 1.040 | 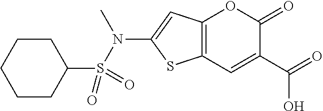 [SMILES: CN(c1cc2oc(=O)c(C(=O)O)cc2s1)S(=O)(=O)C1CCCCC1] | 2-(Cyclohexanesulfonyl- methyl-amino)-5-oxo-5H- thieno[3,2-b]pyran-6- carboxylic acid | NT |
| 1.041 | 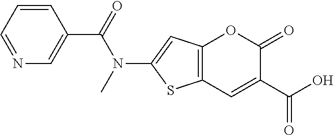 [SMILES: CN(C(=O)c1cccnc1)c1cc2oc(=O)c(C(=O)O)cc2s1] | 2-[Methyl-(pyridine-3- carbonyl)-amino]-5-oxo-5H- thieno[3,2-b]pyran-6- carboxylic acid | NT |
| 1.042 | 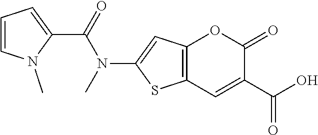 [SMILES: CN(C(=O)c1cccn1C)c1cc2oc(=O)c(C(=O)O)cc2s1] | 2-[Methyl-(1-methyl-1H- pyrrole-2-carbonyl)-amino]-5- oxo-5H-thieno[3,2-b]pyran-6- carboxylic acid | NT |
| 1.043 | 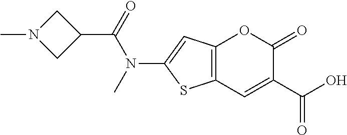 [SMILES: CN1CC(C(=O)N(C)c2cc3oc(=O)c(C(=O)O)cc3s2)C1] | 2-[Methyl-(1-methyl- azetidine-3-carbonyl)-amino]- 5-oxo-5H-thieno[3,2-b]pyran 6-carboxylic acid | NT |
| 1.044 | 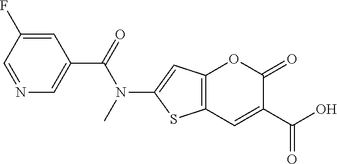 [SMILES: CN(C(=O)c1cncc(F)c1)c1cc2oc(=O)c(C(=O)O)cc2s1] | 2-[(5-Fluoro-pyridine-3- carbonyl)-methyl-amino]-5- oxo-5H-thieno[3,2-b]pyran-6- carboxylic acid | NT |
| 1.045 | 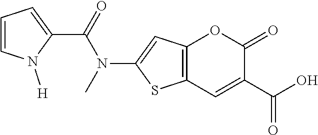 [SMILES: CN(C(=O)c1ccc[nH]1)c1cc2oc(=O)c(C(=O)O)cc2s1] | 2-[Methyl-(1H-pyrrole-2- carbonyl)-amino]-5-oxo-5H- thieno[3,2-b]pyran-6- carboxylic acid | NT |
| 1.046 | 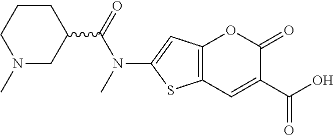 [SMILES: CN1CCCC(C(=O)N(C)c2cc3oc(=O)c(C(=O)O)cc3s2)C1] | 2-[Methyl-(1-methyl- piperidine-3-carbonyl)- amino]-5-oxo-5H-thieno[3,2- b]pyran-6-carboxylic acid | NT |
| 1.047 | 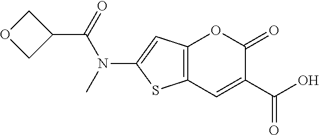 [SMILES: CN(C(=O)C1COC1)c1cc2oc(=O)c(C(=O)O)cc2s1] | 2-[Methyl-(oxetane-3- carbonyl)-amino]-5-oxo-5H- thieno[3,2-b]pyran-6- carboxylic acid | NT |
| 1.048 | 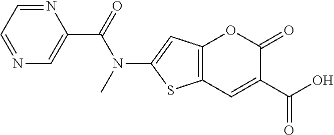 [SMILES: CN(C(=O)c1cnccn1)c1cc2oc(=O)c(C(=O)O)cc2s1] | 2-[Methyl-(pyrazine-2- carbonyl)-amino]-5-oxo-5H- thieno[3,2-b]pyran-6- carboxylic acid | NT |
| 1.049 | 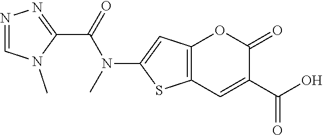 [SMILES: CN(C(=O)c1nncn1C)c1cc2oc(=O)c(C(=O)O)cc2s1] | 2-[Methyl-(4-methyl-4H- [1,2,4]triazole-3-carbonyl)- amino]-5-oxo-5H-thieno[3,2- b]pyran-6-carboxylic acid | NT |
| 1.050 | 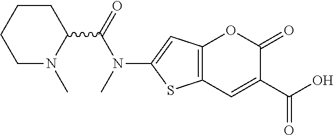 [SMILES: CN(C(=O)C1CCCCN1C)c1cc2oc(=O)c(C(=O)O)cc2s1] | 2-[Methyl-(1-methyl- piperidine-2-carbonyl)- amino]-5-oxo-5H-thieno[3,2- b]pyran-6-carboxylic acid | NT |
| 1.051 | 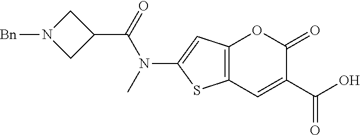 [SMILES: CN(C(=O)C1CN(Cc2ccccc2)C1)c1cc2oc(=O)c(C(=O)O)cc2s1] | 2- [(1-Benzyl-azetidine-3- carbonyl)-methyl-amino]-5- oxo-5H-thieno[3,2-b]pyran-6- carboxylic acid | NT |
| 1.052 | 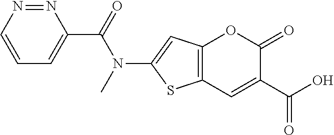 [SMILES: CN(C(=O)c1cccnn1)c1cc2oc(=O)c(C(=O)O)cc2s1] | 2-[Methyl-(pyridazine-3- carbonyl)-amino]-5-oxo-5H- thieno[3,2-b]pyran-6- carboxylic acid | NT |
| 1.053 | 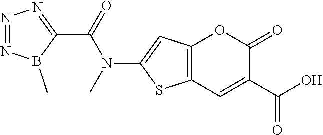 [SMILES: CB1N=NN=C1C(=O)N(C)c1cc2oc(=O)c(C(=O)O)cc2s1] | 2-[Methyl-(1-methyl-1H- tetrazole-5-carbonyl)-amino]- 5-oxo-5H-thieno[3,2-b]pyran- 6-carboxylic acid | NT |
| 1.054 | 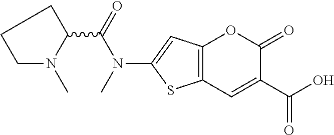 [SMILES: CN(C(=O)C1CCCN1C)c1cc2oc(=O)c(C(=O)O)cc2s1] | 2-[Methyl-(1-methyl- pyrrolidine-2-carbonyl)- amino]-5-oxo-5H-thieno[3,2- b]pyran-6-carboxylic acid | NT |
| 1.055 | 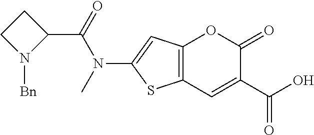 [SMILES: CN(C(=O)C1CCN1Cc1ccccc1)c1cc2oc(=O)c(C(=O)O)cc2s1] | 2-[(1-Benzyl-azetidine-2- carbonyl)-methyl-amino]-5- oxo-5H-thieno[3,2-b]pyran-6- carboxylic acid | NT |
| 1.056 | 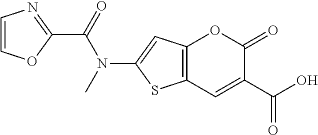 [SMILES: CN(C(=O)c1ncco1)c1cc2oc(=O)c(C(=O)O)cc2s1] | 2-[Methyl-(oxazole-2- carbonyl)-amino]-5-oxo-5H- thieno[3,2-b]pyran-6- carboxylic acid | NT |
| 1.057 | 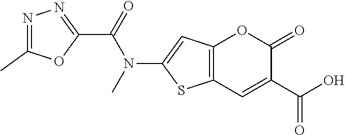 [SMILES: Cc1nnc(C(=O)N(C)c2cc3oc(=O)c(C(=O)O)cc3s2)o1] | 2-[Methyl-(5-methyl- [1,3,4]oxadiazole-2- carbonyl)-amino]-5-oxo-5H- thieno[3,2-b]pyran-6- carboxylic acid | NT |
| 1.058 | 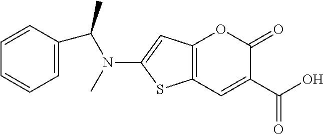 [SMILES: C[C@H](c1ccccc1)N(C)c1cc2oc(=O)c(C(=O)O)cc2s1] | 2-[Methyl-(1-phenyl-ethyl)- amino]-5-oxo-5H-thieno[3,2- b]pyran-6-carboxylic acid | NT |
| 1.059 | 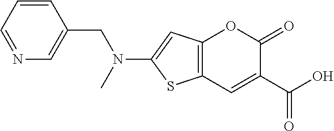 [SMILES: CN(Cc1cccnc1)c1cc2oc(=O)c(C(=O)O)cc2s1] | 2-(Methyl-pyridin-3- ylmethyl-amino)-5-oxo-5H- thieno[3,2-b]pyran-6- carboxylic acid | NT |
| 1.060 | 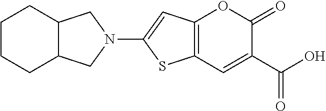 [SMILES: O=C(O)c1cc2sc(N3CC4CCCCC4C3)cc2oc1=O] | 2-(Octahydro-isoindol-2-yl)- 5-oxo-5H-thieno[3,2-b]pyran- 6-carboxylic acid | NT |
| 1.061 | 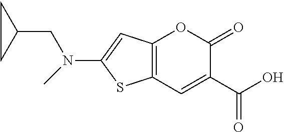 [SMILES: CN(CC1CC1)c1cc2oc(=O)c(C(=O)O)cc2s1] | 2-(Cyclopropylmethyl- methyl-amino)-5-oxo-5H- thieno[3,2-b]pyran-6- carboxylic acid | NT |
| 1.062 | 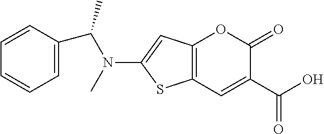 [SMILES: C[C@@H](c1ccccc1)N(C)c1cc2oc(=O)c(C(=O)O)cc2s1] | 2-[Methyl-(1-phenyl-ethyl)- amino]-5-oxo-5H-thieno[3,2- b]pyran-6-carboxylic acid | NT |
| 1.063 | 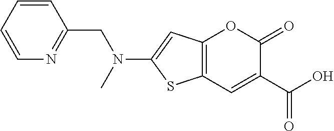 [SMILES: CN(Cc1ccccn1)c1cc2oc(=O)c(C(=O)O)cc2s1] | 2-(Methyl-pyridin-2- ylmethyl-amino)-5-oxo-5H- thieno[3,2-b]pyran-6- carboxylic acid | NT |
| 1.064 | 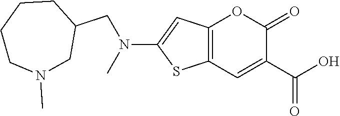 [SMILES: CN1CCCCC(CN(C)c2cc3oc(=O)c(C(=O)O)cc3s2)C1] | 2-[Methyl-(1-methyl-azepan- 3-ylmethyl)-amino]-5-oxo- 5H-thieno[3,2-b]pyran-6- carboxylic acid | NT |
| 1.065 | 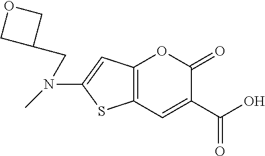 [SMILES: CN(CC1COC1)c1cc2oc(=O)c(C(=O)O)cc2s1] | 2-(Methyl-oxetan-3-ylmethyl- amino)-5-oxo-5H-thieno[3,2- b]pyran-6-carboxylic acid | NT |
| 1.066 | 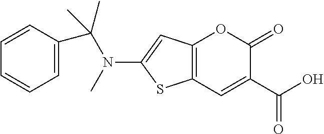 [SMILES: CN(c1cc2oc(=O)c(C(=O)O)cc2s1)C(C)(C)c1ccccc1] | 2-[Methyl-(1-methyl-1- phenyl-ethyl)-amino]-5-oxo- 5H-thieno[3,2-b]pyran-6- carboxylic acid | NT |
| 1.067 | 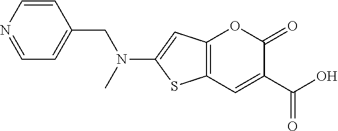 [SMILES: CN(Cc1ccncc1)c1cc2oc(=O)c(C(=O)O)cc2s1] | 2-(Methyl-pyridin-4- ylmethyl-amino)-5-oxo-5H- thieno[3,2-b]pyran-6- carboxylic acid | NT |
| 1.068 | 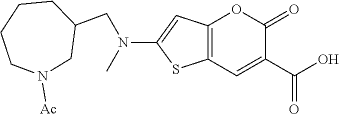 [SMILES: CC(=O)N1CCCCC(CN(C)c2cc3oc(=O)c(C(=O)O)cc3s2)C1] | 2-[(1-Acetyl-azepan-3- ylmethyl)-methyl-amino]-5- oxo-5H-thieno[3,2-b]pyran-6- carboxylic acid | NT |
| 1.069 | 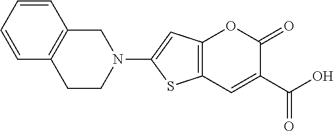 [SMILES: O=C(O)c1cc2sc(N3CCc4ccccc4C3)cc2oc1=O] | 2-(3,4-Dihydro-1H- isoquinolin-2-yl)-5-oxo-5H- thieno[3,2-b]pyran-6- carboxylic acid | NT |
| 1.070 | 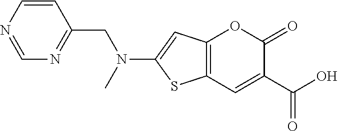 [SMILES: CN(Cc1ccncn1)c1cc2oc(=O)c(C(=O)O)cc2s1] | 2-(Methyl-pyrimidin-4- ylmethyl-amino)-5-oxo-5H- thieno[3,2-b]pyran-6- carboxylic acid | NT |
| 1.071 | 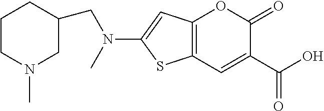 [SMILES: CN1CCCC(CN(C)c2cc3oc(=O)c(C(=O)O)cc3s2)C1] | 2-[Methyl-(1-methyl- piperidin-3-ylmethyl)-amino]- 5-oxo-5H-thieno[3,2-b]pyran- 6-carboxylic acid | NT |
| 1.072 | 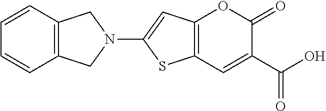 [SMILES: O=C(O)c1cc2sc(N3Cc4ccccc4C3)cc2oc1=O] | 2-(1,3-Dihydro-isoindol-2-yl)- 5-oxo-5H-thieno[3,2-b]pyran- 6-carboxylic acid | NT |
| 1.073 | 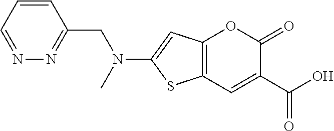 [SMILES: CN(Cc1cccnn1)c1cc2oc(=O)c(C(=O)O)cc2s1] | 2-(Methyl-pyridazin-3- ylmethyl-amino)-5-oxo-5H- thieno[3,2-b]pyran-6- carboxylic acid | NT |
| 1.074 | 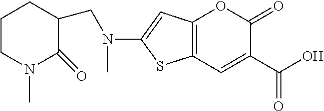 [SMILES: CN1CCCC(CN(C)c2cc3oc(=O)c(C(=O)O)cc3s2)C1=O] | 2-[Methyl-(1-methyl-2-oxo- piperidin-3-ylmethyl)-amino]- 5-oxo-5H-thieno[3,2-b]pyran- 6-carboxylic acid | NT |
| 1.075 | 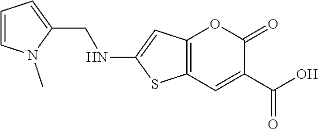 [SMILES: Cn1cccc1CNc1cc2oc(=O)c(C(=O)O)cc2s1] | 2-[(1-Methyl-1H-pyrrol-2- ylmethyl)-amino]-5-oxo-5H- thieno[3,2-b]pyran-6- carboxylic acid | NT |
| 1.076 | 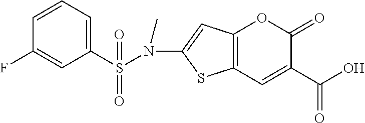 [SMILES: CN(c1cc2oc(=O)c(C(=O)O)cc2s1)S(=O)(=O)c1cccc(F)c1] | 2-[(3-Fluoro- benzenesulfonyl)-methyl- amino]-5-oxo-5H-thieno[3,2- b]pyran-6-carboxylic acid | NT |
| 1.077 | 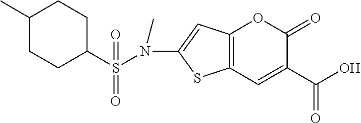 [SMILES: CC1CCC(S(=O)(=O)N(C)c2cc3oc(=O)c(C(=O)O)cc3s2)CC1] | 2-[Methyl-(4-methyl- cyclohexanesulfonyl)-amino]- 5-oxo-5H-thieno[3,2-b]pyran- 6-carboxylic acid | NT |
| 1.078 | 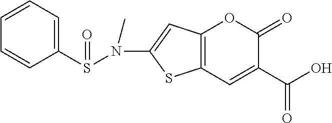 [SMILES: CN(c1cc2oc(=O)c(C(=O)O)cc2s1)S(=O)c1ccccc1] | 2-(Benzenesulfinyl-methyl- amino)-5-oxo-5H-thieno[3,2- b]pyran-6-carboxylic acid | NT |
| 1.079 | 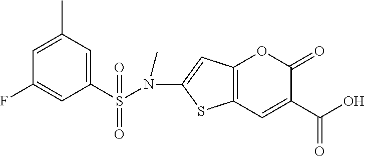 [SMILES: Cc1cc(F)cc(S(=O)(=O)N(C)c2cc3oc(=O)c(C(=O)O)cc3s2)c1] | 2-[(3-Fluoro-5-methyl- benzenesulfonyl)-methyl- amino]-5-oxo-5H-thieno[3,2- b]pyran-6-carboxylic acid | NT |
| 1.080 | 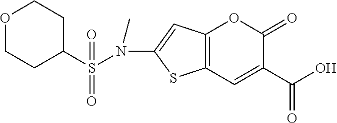 [SMILES: CN(c1cc2oc(=O)c(C(=O)O)cc2s1)S(=O)(=O)C1CCOCC1] | 2-[Methyl-(tetrahydro-pyran- 4-sulfonyl)-amino]-5-oxo-5H- thieno[3,2-b]pyran-6- carboxylic acid | NT |
| 1.081 | 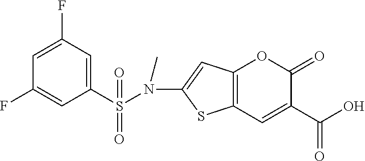 [SMILES: CN(c1cc2oc(=O)c(C(=O)O)cc2s1)S(=O)(=O)c1cc(F)cc(F)c1] | 2-[(3,5-Difluoro- benzenesulfonyl)-methyl- amino]-5-oxo-5H-thieno[3,2- b]pyran-6-carboxylic acid | NT |
| 1.082 | 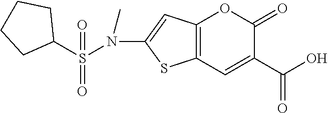 [SMILES: CN(c1cc2oc(=O)c(C(=O)O)cc2s1)S(=O)(=O)C1CCCC1] | 2-(Cyclopentanesulfonyl- methyl-amino)-5-oxo-5H- thieno[3,2-b]pyran-6- carboxylic acid | NT |
| 1.083 | 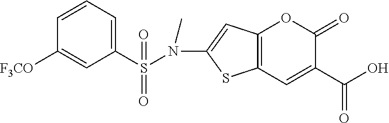 [SMILES: CN(c1cc2oc(=O)c(C(=O)O)cc2s1)S(=O)(=O)c1cccc(OC(F)(F)F)c1] | 2-[Methyl-(3- trifluoromethoxy- benzenesulfonyl)-amino]-5- oxo-5H-thieno[3,2-b]pyran-6- carboxylic acid | NT |
| 1.084 | 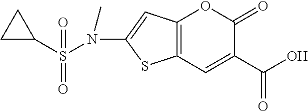 [SMILES: CN(c1cc2oc(=O)c(C(=O)O)cc2s1)S(=O)(=O)C1CC1] | 2-(Cyclopropanesulfonyl- methyl-amino)-5-oxo-5H- thieno[3,2-b]pyran-6- carboxylic acid | NT |
| 1.085 | 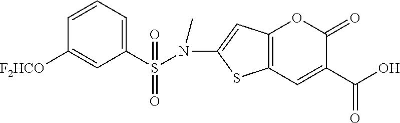 [SMILES: CN(c1cc2oc(=O)c(C(=O)O)cc2s1)S(=O)(=O)c1cccc(OC(F)F)c1] | 2-[(3-Difluoromethoxy- benzenesulfonyl)-methyl- amino]-5-oxo-5H-thieno[3,2- b]pyran-6-carboxylic acid | NT |
| 1.086 | 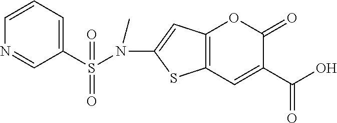 [SMILES: CN(c1cc2oc(=O)c(C(=O)O)cc2s1)S(=O)(=O)c1cccnc1] | 2-[Methyl-(pyridine-3- sulfonyl)-amino]-5-oxo-5H- thieno[3,2-b]pyran-6- carboxylic acid | NT |
| 1.087 | 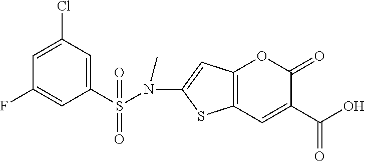 [SMILES: CN(c1cc2oc(=O)c(C(=O)O)cc2s1)S(=O)(=O)c1cc(F)cc(Cl)c1] | 2-[(3-Chloro-5-fluoro- benzenesulfonyl)-methyl- amino]-5-oxo-5H-thieno[3,2- b]pyran-6-carboxylic acid | NT |
| 1.088 | 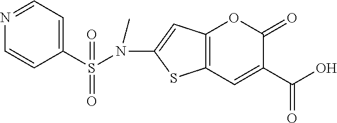 [SMILES: CN(c1cc2oc(=O)c(C(=O)O)cc2s1)S(=O)(=O)c1ccncc1] | 2-[Methyl-(pyridine-4- sulfonyl)-amino]-5-oxo-5H- thieno[3,2-b]pyran-6- carboxylic acid | NT |
| 1.089 | 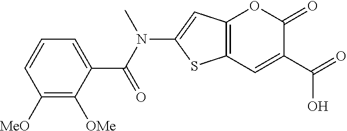 [SMILES: COc1cccc(C(=O)N(C)c2cc3oc(=O)c(C(=O)O)cc3s2)c1OC] | 2-(2,3-dimethoxy-N- methylbenzamido)-5-oxo-5H- thieno[3,2-b]pyran-6- carboxylic acid | NT |
| 1.090 | 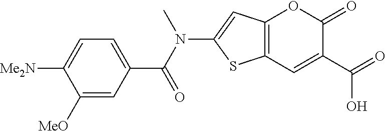 [SMILES: COc1cc(C(=O)N(C)c2cc3oc(=O)c(C(=O)O)cc3s2)ccc1N(C)C] | 2-(4-(dimethylamino)-3- methoxy-N- methylbenzamido)-5-oxo-5H- thieno[3,2-b]pyran-6- carboxylic acid | NT |
| 1.091 | 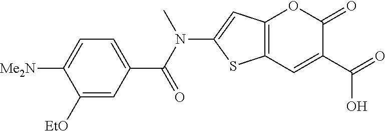 [SMILES: CCOc1cc(C(=O)N(C)c2cc3oc(=O)c(C(=O)O)cc3s2)ccc1N(C)C] | 2-(4-(dimethylamino)-3- ethoxy-N-methylbenzamido)- 5-oxo-5H-thieno[3,2-b]pyran- 6-carboxylic acid | NT |
| 1.092 | 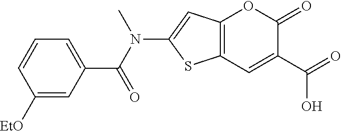 [SMILES: CCOc1cccc(C(=O)N(C)c2cc3oc(=O)c(C(=O)O)cc3s2)c1] | 2-(3-ethoxy-N- methylbenzamido)-5-oxo-5H- thieno[3,2-b]pyran-6- carboxylic acid | NT |
| 1.093 | 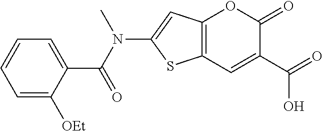 [SMILES: CCOc1ccccc1C(=O)N(C)c1cc2oc(=O)c(C(=O)O)cc2s1] | 2-(2-ethoxy-N- methylbenzamido)-5-oxo-5H- thieno[3,2-b]pyran-6- carboxylic acid | NT |
| 1.094 | 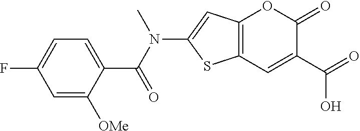 [SMILES: COc1cc(F)ccc1C(=O)N(C)c1cc2oc(=O)c(C(=O)O)cc2s1] | 2-(4-fluoro-2-methoxy-N- methylbenzamido)-5-oxo-5H- thieno[3,2-b]pyran-6- carboxylic acid | A |
| 1.095 | 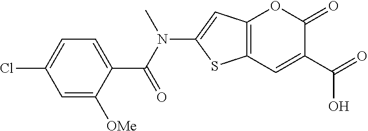 [SMILES: COc1cc(Cl)ccc1C(=O)N(C)c1cc2oc(=O)c(C(=O)O)cc2s1] | 2-(4-chloro-2-methoxy-N- methylbenzamido)-5-oxo-5H- thieno[3,2-b]pyran-6- carboxylic acid | NT |
| 1.096 | 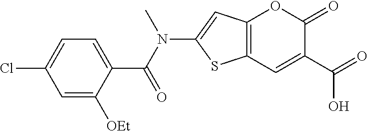 [SMILES: CCOc1cc(Cl)ccc1C(=O)N(C)c1cc2oc(=O)c(C(=O)O)cc2s1] | 2-(4-chloro-2-ethoxy-N- methylbenzamido)-5-oxo-5H- thieno[3,2-b]pyran-6- carboxylic acid | NT |
| 1.097 | 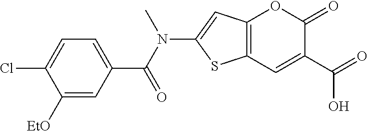 [SMILES: CCOc1cc(C(=O)N(C)c2cc3oc(=O)c(C(=O)O)cc3s2)ccc1Cl] | 2-(4-chloro-3-ethoxy-N- methylbenzamido)-5-oxo-5H- thieno[3,2-b]pyran-6- carboxylic acid | NT |
| 1.098 | 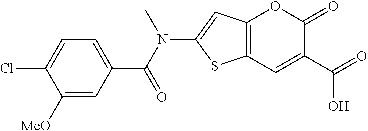 [SMILES: COc1cc(C(=O)N(C)c2cc3oc(=O)c(C(=O)O)cc3s2)ccc1Cl] | 2-(4-chloro-3-methoxy-N- methylbenzamido)-5-oxo-5H- thieno[3,2-b]pyran-6- carboxylic acid | NT |
| 1.099 | 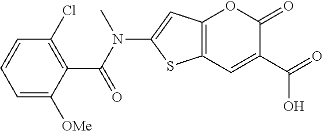 [SMILES: COc1cccc(Cl)c1C(=O)N(C)c1cc2oc(=O)c(C(=O)O)cc2s1] | 2-(2-chloro-6-methoxy-N- methylbenzamido)-5-oxo-5H- thieno[3,2-b]pyran-6- carboxylic acid | NT |
| 1.100 | 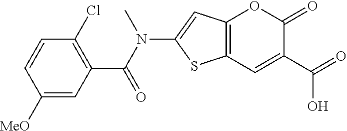 [SMILES: COc1ccc(Cl)c(C(=O)N(C)c2cc3oc(=O)c(C(=O)O)cc3s2)c1] | 2-(2-chloro-5-methoxy-N- methylbenzamido)-5-oxo-5H- thieno[3,2-b]pyran-6- carboxylic acid | NT |

TABLE 2
Compounds and Assay Results (Group 2)

| | | | Cytotoxicity |
|---|---|---|---|
| ID | Structure | Name | [IC₅₀: μM] |
| 2.001 | 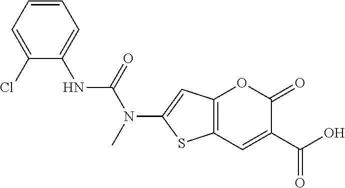 [SMILES: CN(C(=O)Nc1ccccc1Cl)c1cc2oc(=O)c(C(=O)O)cc2s1] | 2-(3-(2-chlorophenyl)-1- methylureido)-5-oxo-5H- thieno[3,2-b]pyran-6- carboxylic acid | A |
| 2.002 | 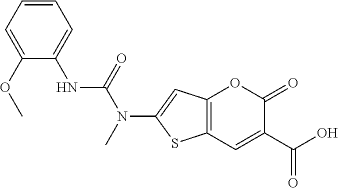 [SMILES: COc1ccccc1NC(=O)N(C)c1cc2oc(=O)c(C(=O)O)cc2s1] | 2-(3-(2-methoxyphenyl)-1- methylureido)-5-oxo-5H- thieno[3,2-b]pyran-6- carboxylic acid | A |
| 2.003 | 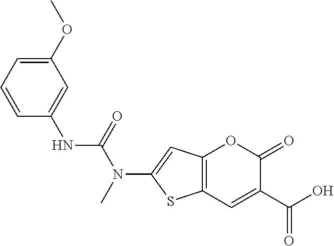 [SMILES: COc1cccc(NC(=O)N(C)c2cc3oc(=O)c(C(=O)O)cc3s2)c1] | 2-(3-(3-methoxyphenyl)-1- methylureido)-5-oxo-5H- thieno[3,2-b]pyran-6- carboxylic acid | A |
| 2.004 | 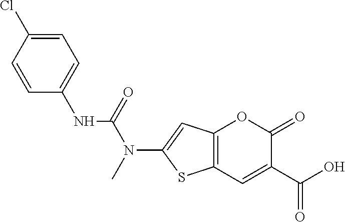 [SMILES: CN(C(=O)Nc1ccc(Cl)cc1)c1cc2oc(=O)c(C(=O)O)cc2s1] | 2-(3-(4-chlorophenyl)-1- methylureido)-5-oxo-5H- thieno[3,2-b]pyran-6- carboxylic acid | A |
| 2.005 | 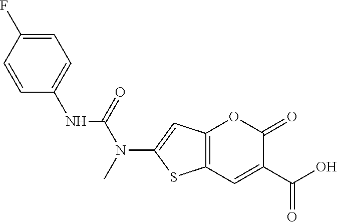 [SMILES: CN(C(=O)Nc1ccc(F)cc1)c1cc2oc(=O)c(C(=O)O)cc2s1] | 2-(3-(4-fluorophenyl)-1- methylureido)-5-oxo-5H- thieno[3,2-b]pyran-6- carboxylic acid | A |
| 2.006 | 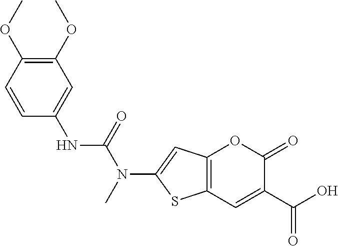 [SMILES: COc1ccc(NC(=O)N(C)c2cc3oc(=O)c(C(=O)O)cc3s2)cc1OC] | 2-(3-(3,4- dimethoxyphenyl)-1- methylureido)-5-oxo-5H- thieno[3,2-b]pyran-6- carboxylic acid | A |
| 2.007 | 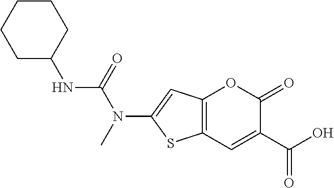 [SMILES: CN(C(=O)NC1CCCCC1)c1cc2oc(=O)c(C(=O)O)cc2s1] | 2-(3-cyclohexyl-1- methylureido)-5-oxo-5H- thieno[3,2-b]pyran-6- carboxylic acid | A |
| 2.008 | 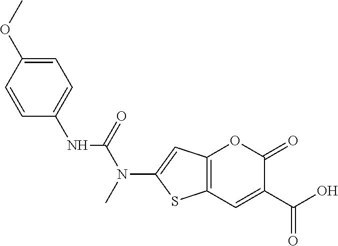 [SMILES: COc1ccc(NC(=O)N(C)c2cc3oc(=O)c(C(=O)O)cc3s2)cc1] | 2-(3-(4-methoxyphenyl)-1- methylureido)-5-oxo-5H- thieno[3,2-b]pyran-6- carboxylic acid | A |
| 2.009 | 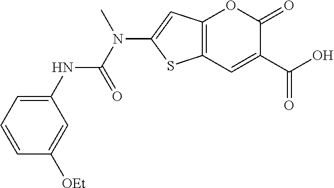 [SMILES: CCOc1cccc(NC(=O)N(C)c2cc3oc(=O)c(C(=O)O)cc3s2)c1] | 2-(3-(3-ethoxyphenyl)-1- methylureido)-5-oxo-5H- thieno[3,2-b]pyran-6- carboxylic acid | A |
| 2.010 | 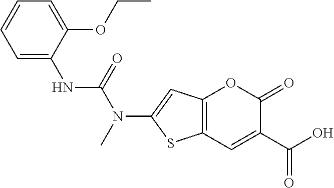 [SMILES: CCOc1ccccc1NC(=O)N(C)c1cc2oc(=O)c(C(=O)O)cc2s1] | 2-(3-(2-ethoxyphenyl)-1- methylureido)-5-oxo-5H- thieno[3,2-b]pyran-6- carboxylic acid | A |
| 2.011 | 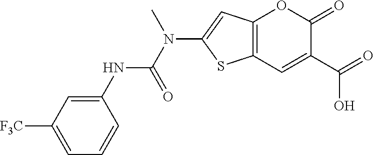 [SMILES: CN(C(=O)Nc1cccc(C(F)(F)F)c1)c1cc2oc(=O)c(C(=O)O)cc2s1] | 2-(1-methyl-3-(3- (trifluoromethyl)phenyl) ureido)-5-oxo-5H-thieno[3,2- b]pyran-6-carboxylic acid | A |
| 2.012 | 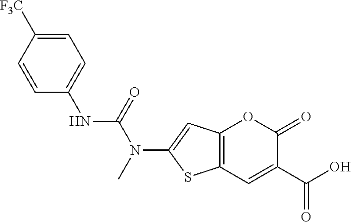 [SMILES: CN(C(=O)Nc1ccc(C(F)(F)F)cc1)c1cc2oc(=O)c(C(=O)O)cc2s1] | 2-(1-methyl-3-(4- (trifluoromethyl)phenyl) ureido)-5-oxo-5H-thieno[3,2- b]pyran-6-carboxylic acid | A |
| 2.013 | 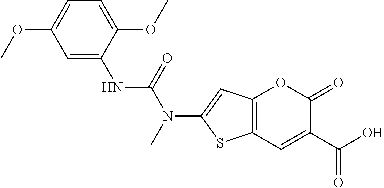 [SMILES: COc1ccc(OC)c(NC(=O)N(C)c2cc3oc(=O)c(C(=O)O)cc3s2)c1] | 2-(3-(2,5- dimethoxyphenyl)-1- methylureido)-5-oxo-5H- thieno[3,2-b]pyran-6- carboxylic acid | A |
| 2.014 | 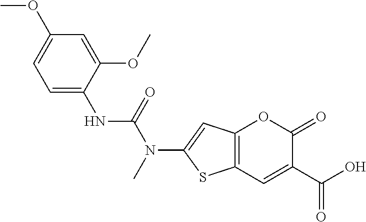 [SMILES: COc1ccc(NC(=O)N(C)c2cc3oc(=O)c(C(=O)O)cc3s2)c(OC)c1] | 2-(3-(2,4- dimethoxyphenyl)-1- methylureido)-5-oxo-5H- thieno[3,2-b]pyran-6- carboxylic acid | A |
| 2.015 | 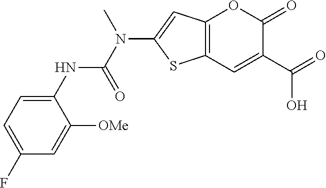 [SMILES: COc1cc(F)ccc1NC(=O)N(C)c1cc2oc(=O)c(C(=O)O)cc2s1] | 2-(3-(4-fluoro-2- methoxyphenyl)-1- methylureido)-5-oxo-5H- thieno[3,2-b]pyran-6- carboxylic acid | A |
| 2.016 | 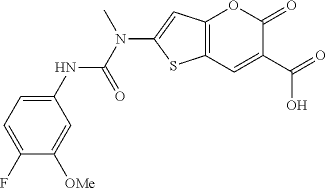 [SMILES: COc1cc(NC(=O)N(C)c2cc3oc(=O)c(C(=O)O)cc3s2)ccc1F] | 2-(3-(4-fluoro-3- methoxyphenyl)-1- methylureido)-5-oxo-5H- thieno[3,2-b]pyran-6- carboxylic acid | A |
| 2.017 | 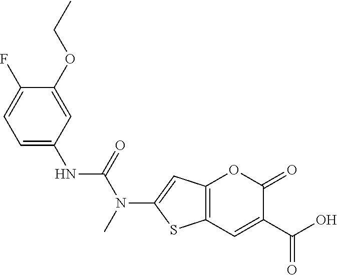 [SMILES: CCOc1cc(NC(=O)N(C)c2cc3oc(=O)c(C(=O)O)cc3s2)ccc1F] | 2-(3-(3-ethoxy-4- fluorophenyl)-1- methylureido)-5-oxo-5H- thieno[3,2-b]pyran-6- carboxylic acid | A |
| 2.018 | 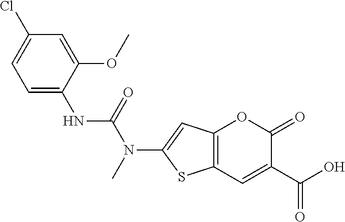 [SMILES: COc1cc(Cl)ccc1NC(=O)N(C)c1cc2oc(=O)c(C(=O)O)cc2s1] | 2-(1-methyl-3-(4- (trifluoromethoxy) phenyl)ureido)-5-oxo-5H- thieno[3,2-b]pyran-6- carboxylic acid | A |
| 2.019 | 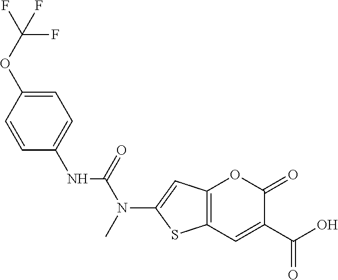 [SMILES: CN(C(=O)Nc1ccc(OC(F)(F)F)cc1)c1cc2oc(=O)c(C(=O)O)cc2s1] | 2-(1-methyl-3-(4- (trifluoromethoxy) phenyl)ureido)-5-oxo-5H- thieno[3,2-b]pyran-6- carboxylic acid | A |
| 2.020 | 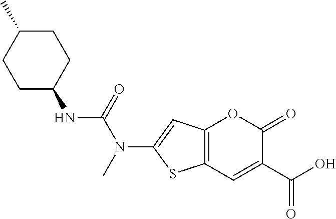 [SMILES: CN(C(=O)N[C@H]1CC[C@H](C)CC1)c1cc2oc(=O)c(C(=O)O)cc2s1] | 2-(1-methyl-3-((1r,4r)-4- methylcyclohexyl)ureido)- 5-oxo-5H-thieno[3,2- b]pyran-6-carboxylic acid | A |
| 2.021 | 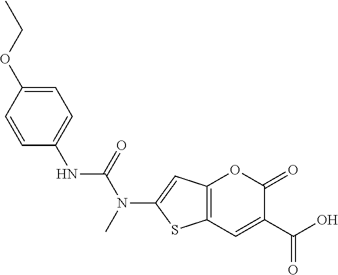 [SMILES: CCOc1ccc(NC(=O)N(C)c2cc3oc(=O)c(C(=O)O)cc3s2)cc1] | 2-(3-(4-ethoxyphenyl)-1- methylureido)-5-oxo-5H- thieno[3,2-b]pyran-6- carboxylic acid | A |
| 2.022 | 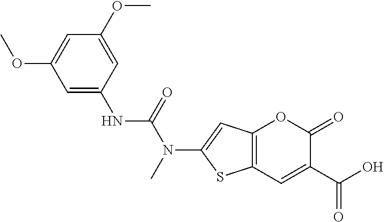 [SMILES: COc1cc(NC(=O)N(C)c2cc3oc(=O)c(C(=O)O)cc3s2)cc(OC)c1] | 2-(3-(3,5- dimethoxyphenyl)-1- methylureido)-5-oxo-5H- thieno[3,2-b]pyran-6- carboxylic acid | A |
| 2.023 | 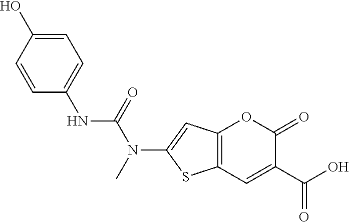 [SMILES: CN(C(=O)Nc1ccc(O)cc1)c1cc2oc(=O)c(C(=O)O)cc2s1] | 2-(3-(4-hydroxyphenyl)-1- methylureido)-5-oxo-5H- thieno[3,2-b]pyran-6- carboxylic acid | A |
| 2.024 | 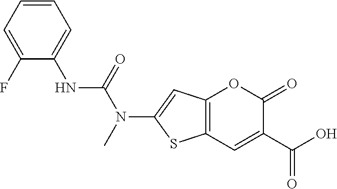 [SMILES: CN(C(=O)Nc1ccccc1F)c1cc2oc(=O)c(C(=O)O)cc2s1] | 2-(3-(2-fluorophenyl)-1- methylureido)-5-oxo-5H- thieno[3,2-b]pyran-6- carboxylic acid | A |
| 2.025 | 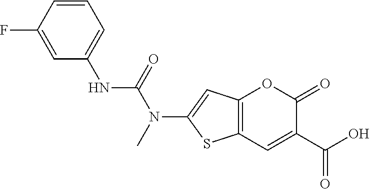 [SMILES: CN(C(=O)Nc1cccc(F)c1)c1cc2oc(=O)c(C(=O)O)cc2s1] | 2-(3-(3-fluorophenyl)-1- methylureido)-5-oxo-5H- thieno[3,2-b]pyran-6- carboxylic acid | A |
| 2.026 | 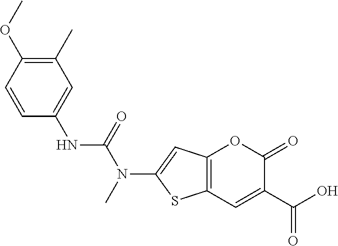 [SMILES: COc1ccc(NC(=O)N(C)c2cc3oc(=O)c(C(=O)O)cc3s2)cc1C] | 2-(3-(4-methoxy-3- methylphenyl)-1- methylureido)-5-oxo-5H- thieno[3,2-b]pyran-6- carboxylic acid | A |
| 2.027 | 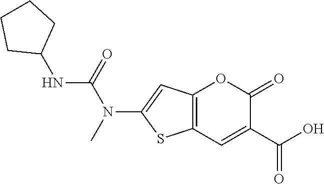 [SMILES: CN(C(=O)NC1CCCC1)c1cc2oc(=O)c(C(=O)O)cc2s1] | 2-(3-cyclopentyl-1- methylureido)-5-oxo-5H- thieno[3,2-b]pyran-6- carboxylic acid | A |
| 2.028 | 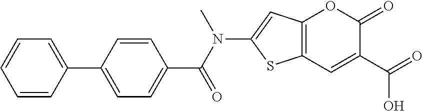 [SMILES: CN(C(=O)c1ccc(-c2ccccc2)cc1)c1cc2oc(=O)c(C(=O)O)cc2s1] | 2-(N-methyl-[1,1′- biphenyl]-4-carboxamido)- 5 -oxo-5H-thieno[3,2- b]pyran-6-carboxylic acid | A |
| 2.029 | 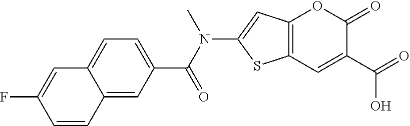 [SMILES: CN(C(=O)c1ccc2cc(F)ccc2c1)c1cc2oc(=O)c(C(=O)O)cc2s1] | 2-(6-fluoro-N-methyl-2- naphthamido)-5-oxo-5H- thieno[3,2-b]pyran-6- carboxylic acid | A |
| 2.030 | 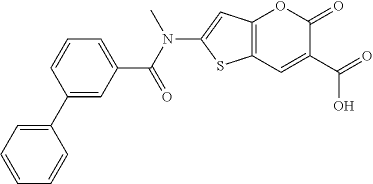 [SMILES: CN(C(=O)c1cccc(-c2ccccc2)c1)c1cc2oc(=O)c(C(=O)O)cc2s1] | 2-(N-methyl-[1,1′- biphenyl]-3-carboxamido)- 5-oxo-5H-thieno[3,2- b]pyran-6-carboxylic acid | A |
| 2.031 | 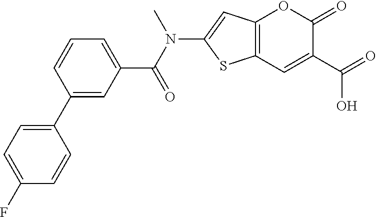 [SMILES: CN(C(=O)c1cccc(-c2ccc(F)cc2)c1)c1cc2oc(=O)c(C(=O)O)cc2s1] | 2-(4′-fluoro-N-methyl-[1,1′- biphenyl]-3-carboxamido)- 5-oxo-5H-thieno[3,2- b]pyran-6-carboxylic acid | A |
| 2.032 | 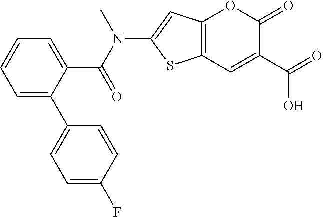 [SMILES: CN(C(=O)c1ccccc1-c1ccc(F)cc1)c1cc2oc(=O)c(C(=O)O)cc2s1] | 2-(4′-fluoro-N-methyl-[1,1′- biphenyl]-2-carboxamido)- 5-oxo-5H-thieno[3,2- b]pyran-6-carboxylic acid | A |
| 2.033 | 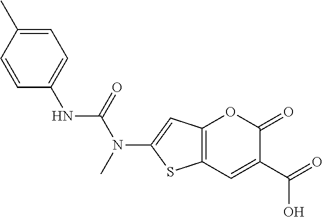 [SMILES: Cc1ccc(NC(=O)N(C)c2cc3oc(=O)c(C(=O)O)cc3s2)cc1] | 2-(1-methyl-3-(p- tolyl)ureido)-5-oxo-5H- thieno[3,2-b]pyran-6- carboxylic acid | A |
| 2.034 | 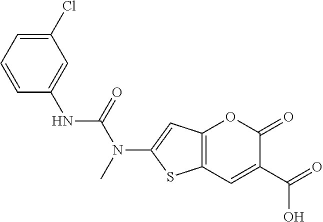 [SMILES: CN(C(=O)Nc1cccc(Cl)c1)c1cc2oc(=O)c(C(=O)O)cc2s1] | 2-(3-(3-chlorophenyl)-1- methylureido)-5-oxo-5H- thieno[3,2-b]pyran-6- carboxylic acid | A |
| 2.035 | 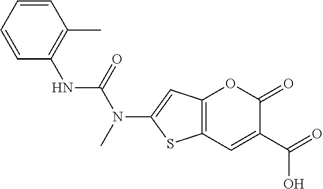 [SMILES: Cc1ccccc1NC(=O)N(C)c1cc2oc(=O)c(C(=O)O)cc2s1] | 2-(1-methyl-3-(o- tolyl)ureido)-5-oxo-5H- thieno[3,2-b]pyran-6- carboxylic acid | A |
| 2.036 | 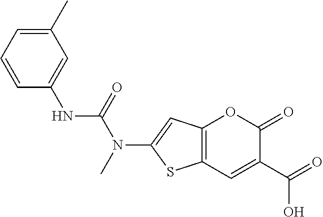 [SMILES: Cc1cccc(NC(=O)N(C)c2cc3oc(=O)c(C(=O)O)cc3s2)c1] | 2-(1-methyl-3-(m- tolyl)ureido)-5-oxo-5H- thieno[3,2-b]pyran-6- carboxylic acid | A |
| 2.037 | 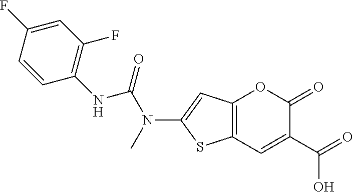 [SMILES: CN(C(=O)Nc1ccc(F)cc1F)c1cc2oc(=O)c(C(=O)O)cc2s1] | 2-(3-(2,4-difluorophenyl)- 1-methy lureido)-5-oxo-5H- thieno[3,2-b]pyran-6- carboxylic acid | A |
| 2.038 | 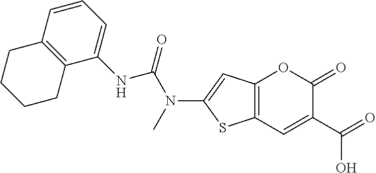 [SMILES: CN(C(=O)Nc1cccc2c1CCCC2)c1cc2oc(=O)c(C(=O)O)cc2s1] | 2-(1-methyl-3-(5,6,7,8- tetrahydronaphthalen-1- yl)ureido)-5-oxo-5H- thieno[3,2-b]pyran-6- carboxylic acid | A |
| 2.039 | 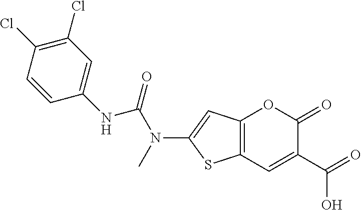 [SMILES: CN(C(=O)Nc1ccc(Cl)c(Cl)c1)c1cc2oc(=O)c(C(=O)O)cc2s1] | 2-(3-(3,4-difluorophenyl)- 1-methylureido)-5-oxo-5H- thieno[3,2-b]pyran-6- carboxylic acid | A |
| 2.040 | 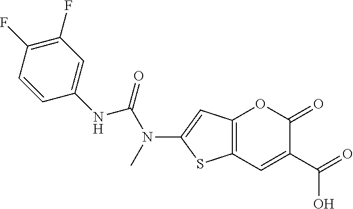 [SMILES: CN(C(=O)Nc1ccc(F)c(F)c1)c1cc2oc(=O)c(C(=O)O)cc2s1] | 2-(3-(3,4-difluorophenyl)- 1-methylureido)-5-oxo-5H- thieno[3,2-b]pyran-6- carboxylic acid | A |
| 2.041 | 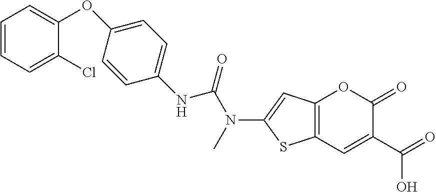 [SMILES: CN(C(=O)Nc1ccc(Oc2ccccc2Cl)cc1)c1cc2oc(=O)c(C(=O)O)cc2s1] | 2-(3-(4-(2- chlorophenoxy)phenyl)-1- methylureido)-5-oxo-5H- thieno[3,2-b]pyran-6- carboxylic acid | A |
| 2.042 | 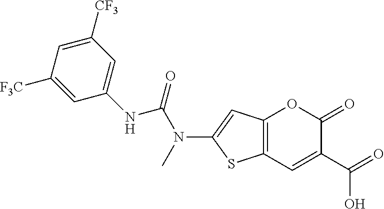 [SMILES: CN(C(=O)Nc1cc(C(F)(F)F)cc(C(F)(F)F)c1)c1cc2oc(=O)c(C(=O)O)cc2s1] | 2-(3-(3,5- bis(trifluoromethyl)phenyl)- 1-methylureido)-5-oxo-5H- thieno[3,2-b]pyran-6- carboxylic acid | A |
| 2.043 | 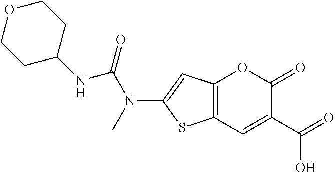 [SMILES: CN(C(=O)NC1CCOCC1)c1cc2oc(=O)c(C(=O)O)cc2s1] | 2-(1-methyl-3-(tetrahydro- 2H-pyran-4-yl)ureido)-5- oxo-5H-thieno[3,2-b]pyran- 6-carboxylic acid | A |
| 2.044 | 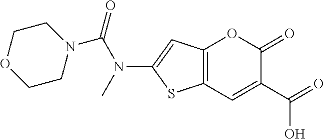 [SMILES: CN(C(=O)N1CCOCC1)c1cc2oc(=O)c(C(=O)O)cc2s1] | 2-(N-methylmorpholine-4- carboxamido)-5-oxo-5H- thieno[3,2-b]pyran-6- carboxylic acid | A |
| 2.045 | 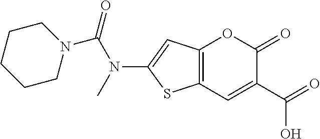 [SMILES: CN(C(=O)N1CCCCC1)c1cc2oc(=O)c(C(=O)O)cc2s1] | 2-(N-methylpiperidine-1- carboxamido)-5-oxo-5H- thieno[3,2-b]pyran-6- carboxylic acid | A |
| 2.046 | 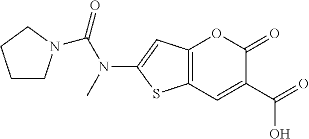 [SMILES: CN(C(=O)N1CCCC1)c1cc2oc(=O)c(C(=O)O)cc2s1] | 2-(N-methylpyrrolidine-1- carboxamido)-5-oxo-5H- thieno[3,2-b]pyran-6- carboxylic acid | A |
| 2.047 | 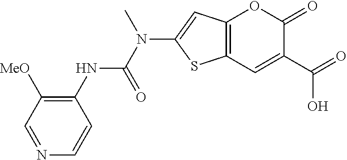 [SMILES: COc1cnccc1NC(=O)N(C)c1cc2oc(=O)c(C(=O)O)cc2s1] | 2-(3-(3-methoxypyridin-4- yl)-1-methylureido)-5-oxo- 5H-thieno[3,2-b]pyran-6- carboxylic acid | A |
| 2.048 | 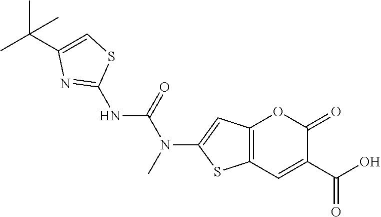 [SMILES: CN(C(=O)Nc1nc(C(C)(C)C)cs1)c1cc2oc(=O)c(C(=O)O)cc2s1] | 2-(3-(4-(tert-butyl)thiazol-2- yl)-1-methylureido)-5-oxo- 5H-thieno[3,2-b]pyran-6- carboxylic acid | A |
| 2.049 | 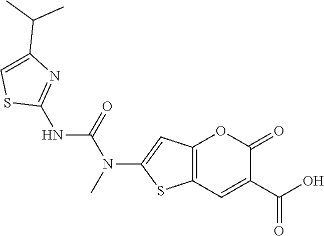 [SMILES: CC(C)c1csc(NC(=O)N(C)c2cc3oc(=O)c(C(=O)O)cc3s2)n1] | 2-(3-(4-isopropylthiazol-2- yl)-1-methylureido)-5-oxo- 5H-thieno[3,2-b]pyran-6- carboxylic acid | A |
| 2.050 | 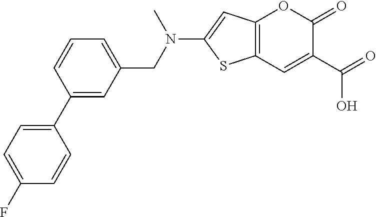 [SMILES: CN(Cc1cccc(-c2ccc(F)cc2)c1)c1cc2oc(=O)c(C(=O)O)cc2s1] | 2-(((4′-fluoro-[1,1′- biphenyl]-3- yl)methyl)(methyl)amino)- 5-oxo-5H-thieno[3,2- b]pyran-6-carboxylic acid | A |
| 2.051 | 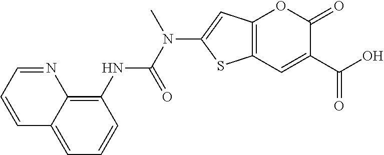 [SMILES: CN(C(=O)Nc1cccc2cccnc12)c1cc2oc(=O)c(C(=O)O)cc2s1] | 2-(1-methyl-3-(quinolin-8- yl)ureido)-5-oxo-5H- thieno[3,2-b]pyran-6- carboxylic acid | NT |
| 2.052 | 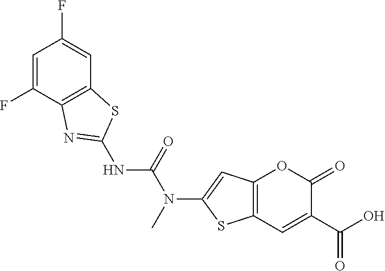 [SMILES: CN(C(=O)Nc1nc2c(F)cc(F)cc2s1)c1cc2oc(=O)c(C(=O)O)cc2s1] | 2-(3-(4,6- difluorobenzo[d]thiazol-2- yl)-1-methylureido)-5-oxo- 5H-thieno[3,2-b]pyran-6- carboxylic acid | NT |
| 2.053 | 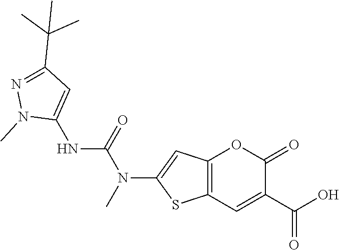 [SMILES: CN(C(=O)Nc1cc(C(C)(C)C)nn1C)c1cc2oc(=O)c(C(=O)O)cc2s1] | 2-(3-(3-(tert-butyl)-1- methyl-1H-pyrazol-5-yl)-1- methylureido)-5-oxo-5H- thieno[3,2-b]pyran-6- carboxylic acid | NT |
| 2.054 | 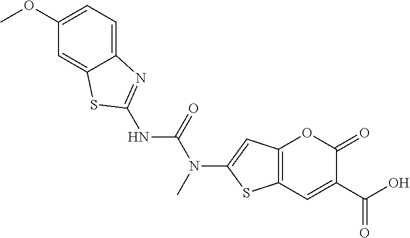 [SMILES: COc1ccc2nc(NC(=O)N(C)c3cc4oc(=O)c(C(=O)O)cc4s3)sc2c1] | 2-(3-(6- methoxybenzo[d]thiazol-2- yl)-1-methylureido)-5-oxo- 5H-thieno[3,2-b]pyran-6- carboxylic acid | NT |
| 2.055 | 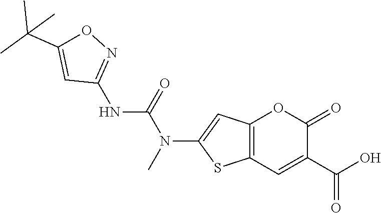 [SMILES: CN(C(=O)Nc1cc(C(C)(C)C)on1)c1cc2oc(=O)c(C(=O)O)cc2s1] | 2-(3-(5-(tert-butyl)isoxazol- 3-yl)-1-methylureido)-5- oxo-5H-thieno[3,2-b]pyran- 6-carboxylic acid | A |
| 2.056 | 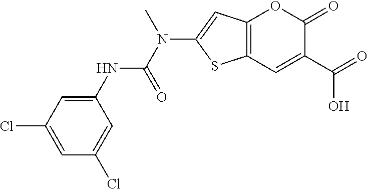 [SMILES: CN(C(=O)Nc1cc(Cl)cc(Cl)c1)c1cc2oc(=O)c(C(=O)O)cc2s1] | 2-(3-(3,5-dichlorophenyl)-1- methylureido)-5-oxo-5H- thieno[3,2-b]pyran-6- carboxylic acid | NT |
| 2.057 | 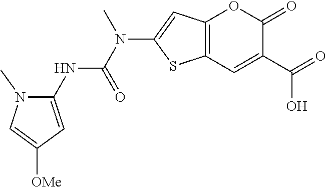 [SMILES: COc1cc(NC(=O)N(C)c2cc3oc(=O)c(C(=O)O)cc3s2)n(C)c1] | 2-(3-(4-methoxy-1-methyl- 1H-pyrrol-2-yl)-1- methylureido)-5-oxo-5H- thieno[3,2-b]pyran-6- carboxylic acid | NT |
| 2.058 | 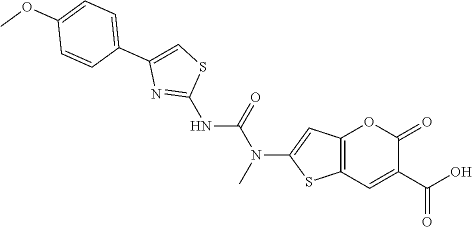 [SMILES: COc1ccc(-c2csc(NC(=O)N(C)c3cc4oc(=O)c(C(=O)O)cc4s3)n2)cc1] | 2-(3-(4-(4- methoxyphenyl)thiazol-2- yl)-1-methylureido)-5-oxo- 5H-thieno[3,2-b]pyran-6- carboxylic acid | NT |
| 2.059 | 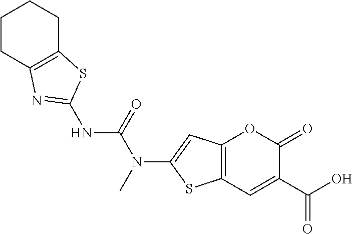 [SMILES: CN(C(=O)Nc1nc2c(s1)CCCC2)c1cc2oc(=O)c(C(=O)O)cc2s1] | 2-(1-methyl-3-(4,5,6,7- tetrahydrobenzo[d]thiazol- 2-yl)ureido)-5-oxo-5H- thieno[3,2-b]pyran-6- carboxylic acid | NT |
| 2.060 | 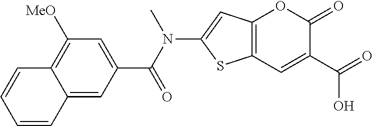 [SMILES: COc1cc(C(=O)N(C)c2cc3oc(=O)c(C(=O)O)cc3s2)cc2ccccc12] | 2-(4-methoxy-N-methyl-2- naphthamido)-5-oxo-5H- thieno[3,2-b]pyran-6- carboxylic acid | NT |
| 2.061 | 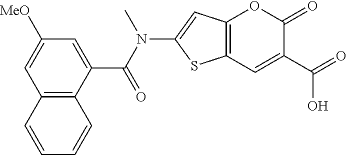 [SMILES: COc1cc(C(=O)N(C)c2cc3oc(=O)c(C(=O)O)cc3s2)c2ccccc2c1] | 2-(3 -methoxy-N -methyl-1- naphthamido)-5-oxo-5H- thieno[3,2-b]pyran-6- carboxylic acid | NT |
| 2.062 | 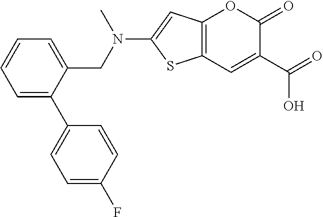 [SMILES: CN(Cc1ccccc1-c1ccc(F)cc1)c1cc2oc(=O)c(C(=O)O)cc2s1] | 2-(((4′-fluoro-[1,1′- biphenyl]-2- yl)methyl)(methyl)amino)- 5 -oxo-5H-thieno[3,2- b]pyran-6-carboxylic acid | NT |
| 2.063 | 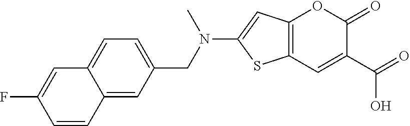 [SMILES: CN(Cc1ccc2cc(F)ccc2c1)c1cc2oc(=O)c(C(=O)O)cc2s1] | 2-(((6-fluoronaphthalen-2- yl)methyl)(methyl)amino)- 5-oxo-5H-thieno[3,2- b]pyran-6-carboxylic acid | NT |
| 2.064 | 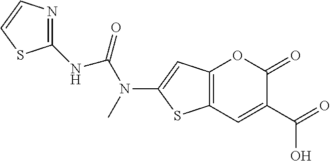 [SMILES: CN(C(=O)Nc1nccs1)c1cc2oc(=O)c(C(=O)O)cc2s1] | 2-(1-methyl-3-(thiazol-2- yl)ureido)-5-oxo-5H- thieno[3,2-b]pyran-6- carboxylic acid | NT |
| 2.065 | 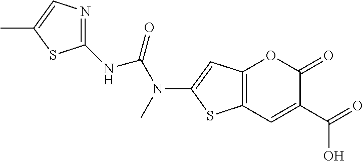 [SMILES: Cc1cnc(NC(=O)N(C)c2cc3oc(=O)c(C(=O)O)cc3s2)s1] | 2-(1-methyl-3-(5- methylthiazol-2-yl)ureido)- 5-oxo-5H-thieno[3,2- b]pyran-6-carboxylic acid | NT |
| 2.066 | 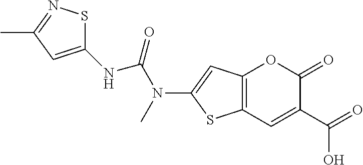 [SMILES: Cc1cc(NC(=O)N(C)c2cc3oc(=O)c(C(=O)O)cc3s2)sn1] | 2-(1-methyl-3-(3- methylisothiazol-5- yl)ureido)-5-oxo-5H- thieno[3,2-b]pyran-6- carboxylic acid | NT |
| 2.067 | 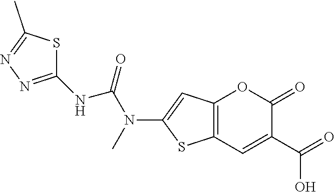 [SMILES: Cc1nnc(NC(=O)N(C)c2cc3oc(=O)c(C(=O)O)cc3s2)s1] | 2-(1-methyl-3-(5-methyl- 1,3,4-thiadiazol-2- yl)ureido)-5-oxo-5H- thieno[3,2-b]pyran-6- carboxylic acid | NT |
| 2.068 | 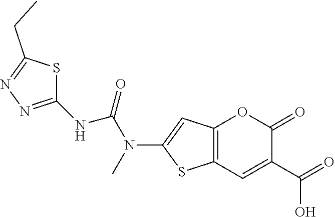 [SMILES: CCc1nnc(NC(=O)N(C)c2cc3oc(=O)c(C(=O)O)cc3s2)s1] | 2-(3-(5-ethyl-1,3,4- thiadiazol-2-yl)-1- methylureido)-5-oxo-5H- thieno[3,2-b]pyran-6- carboxylic acid | NT |
| 2.069 | 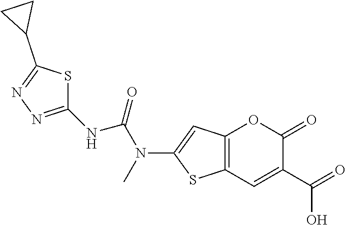 [SMILES: CN(C(=O)Nc1nnc(C2CC2)s1)c1cc2oc(=O)c(C(=O)O)cc2s1] | 2-(3-(5-cyclopropyl-1,3,4- thiadiazol-2-yl)-1- methylureido)-5-oxo-5H- thieno[3,2-b]pyran-6- carboxylic acid | NT |
| 2.070 | 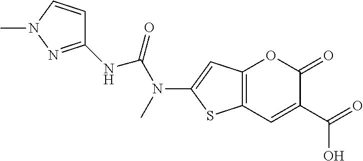 [SMILES: CN(C(=O)Nc1ccn(C)n1)c1cc2oc(=O)c(C(=O)O)cc2s1] | 2-(1-methyl-3-(1-methyl- 1H-pyrazol-3-yl)ureido)-5- oxo-5H-thieno[3,2-b]pyran- 6-carboxylic acid | NT |
| 2.071 | 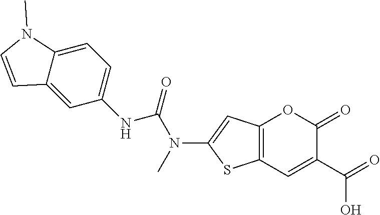 [SMILES: CN(C(=O)Nc1ccc2c(ccn2C)c1)c1cc2oc(=O)c(C(=O)O)cc2s1] | 2-(1-methyl-3-(1methyl- 1H-indol-5-yl)ureido)-5- oxo-5H-thieno[3,2-b]pyran- 6-carboxylic acid | NT |
| 2.072 | 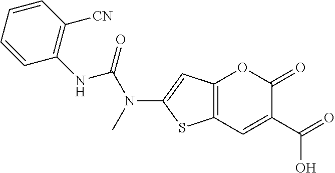 [SMILES: CN(C(=O)Nc1ccccc1C#N)c1cc2oc(=O)c(C(=O)O)cc2s1] | 2-(3-(2-cyanophenyl)-1- methylureido)-5-oxo-5H- thieno[3,2-b]pyran-6- carboxylic acid | NT |
| 2.073 | 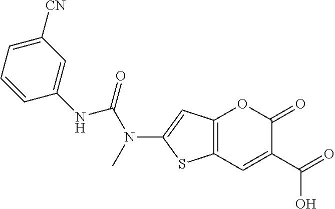 [SMILES: CN(C(=O)Nc1cccc(C#N)c1)c1cc2oc(=O)c(C(=O)O)cc2s1] | 2-(3-(3-cyanophenyl)-1- methylureido)-5-oxo-5H- thieno[3,2-b]pyran-6- carboxylic acid | NT |
| 2.074 | 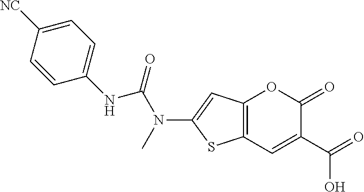 [SMILES: CN(C(=O)Nc1ccc(C#N)cc1)c1cc2oc(=O)c(C(=O)O)cc2s1] | 2-(3-(4-cyanophenyl)-1- methylureido)-5-oxo-5H- thieno[3,2-b]pyran-6- carboxylic acid | NT |
| 2.075 | 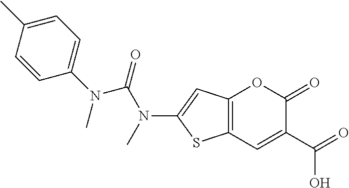 [SMILES: Cc1ccc(N(C)C(=O)N(C)c2cc3oc(=O)c(C(=O)O)cc3s2)cc1] | 2-(1,3-dimethyl-3-(p- tolyl)ureido)-5-oxo-5H- thieno[3,2-b]pyran-6- carboxylic acid | NT |
| 2.076 | 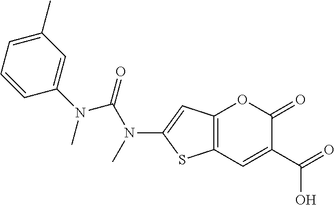 [SMILES: Cc1cccc(N(C)C(=O)N(C)c2cc3oc(=O)c(C(=O)O)cc3s2)c1] | 2-(1,3-dimethyl-3-(m- tolyl)ureido)-5-oxo-5H- thieno[3,2-b]pyran-6- carboxylic acid | NT |
| 2.077 | 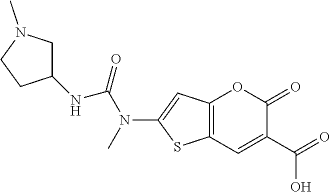 [SMILES: CN1CCC(NC(=O)N(C)c2cc3oc(=O)c(C(=O)O)cc3s2)C1] | 2-(1-methyl-3-(1- methylpyrrolidin-3- yl)ureido)-5-oxo-5H- thieno[3,2-b]pyran-6- carboxylic acid | NT |
| 2.078 | 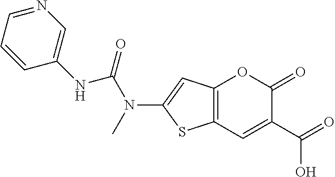 [SMILES: CN(C(=O)Nc1cccnc1)c1cc2oc(=O)c(C(=O)O)cc2s1] | 2-(1-methyl-3-(pyridin-3- yl)ureido)-5-oxo-5H- thieno[3,2-b]pyran-6- carboxylic acid | NT |
| 2.079 | 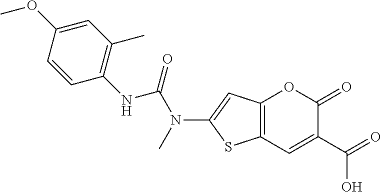 [SMILES: COc1ccc(NC(=O)N(C)c2cc3oc(=O)c(C(=O)O)cc3s2)c(C)c1] | 2-(3-(4-methoxy-2- methylphenyl)-1- methylureido)-5-oxo-5H- thieno[3,2-b]pyran-6- carboxylic acid | NT |
| 2.080 | 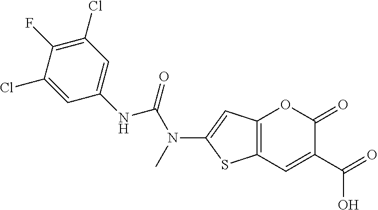 [SMILES: CN(C(=O)Nc1cc(Cl)c(F)c(Cl)c1)c1cc2oc(=O)c(C(=O)O)cc2s1] | 2-(3-(3,5-dichloro-4- fluorophenyl)-1- methylureido)-5-oxo-5H- thieno[3,2-b]pyran-6- carboxylic acid | NT |
| 2.081 | 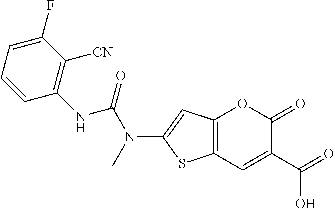 [SMILES: CN(C(=O)Nc1cccc(F)c1C#N)c1cc2oc(=O)c(C(=O)O)cc2s1] | 2-(3-(2-cyano-3- fluorophenyl)-1- methylureido)-5-oxo-5H- thieno[3,2-b]pyran-6- carboxylic acid | NT |
| 2.082 | 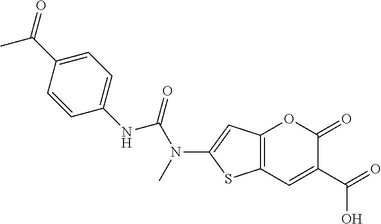 [SMILES: CC(=O)c1ccc(NC(=O)N(C)c2cc3oc(=O)c(C(=O)O)cc3s2)cc1] | 2-(3-(4-acetylphenyl)-1- methylureido)-5-oxo-5H- thieno[3,2-b]pyran-6- carboxylic acid | NT |
| 2.083 | 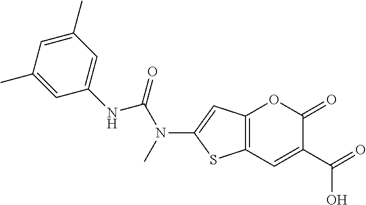 [SMILES: Cc1cc(C)cc(NC(=O)N(C)c2cc3oc(=O)c(C(=O)O)cc3s2)c1] | 2-(3-(3,5-dimethylphenyl)- 1-methylureido)-5-oxo-5H- thieno[3,2-b]pyran-6- carboxylic acid | NT |
| 2.084 | 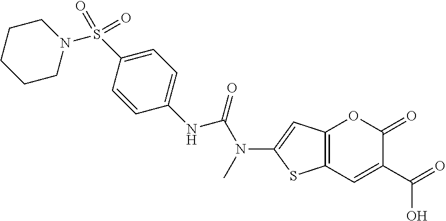 [SMILES: CN(C(=O)Nc1ccc(S(=O)(=O)N2CCCCC2)cc1)c1cc2oc(=O)c(C(=O)O)cc2s1] | 2-(1-methyl-3-(4-(piperidin- 1-ylsulfonyl)phenyl)ureido)- 5-oxo-5H-thieno[3,2- b]pyran-6-carboxylic acid | NT |
| 2.085 | 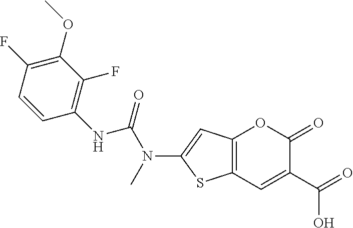 [SMILES: COc1c(F)ccc(NC(=O)N(C)c2cc3oc(=O)c(C(=O)O)cc3s2)c1F] | 2-(3 -(2,4-difluoro-3- methoxyphenyl)-1- methylureido)-5-oxo-5H- thieno[3,2-b]pyran-6- carboxylic acid | NT |
| 2.086 | 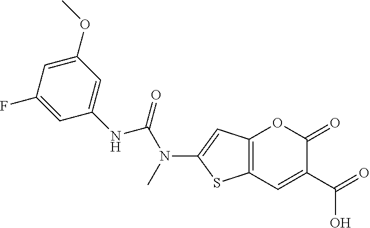 [SMILES: COc1cc(F)cc(NC(=O)N(C)c2cc3oc(=O)c(C(=O)O)cc3s2)c1] | 2-(3-(3-fluoro-5- methoxyphenyl)-1- methylureido)-5-oxo-5H- thieno[3,2-b]pyran-6- carboxylic acid | NT |
| 2.087 | 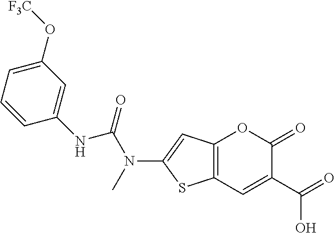 [SMILES: CN(C(=O)Nc1cccc(OC(F)(F)F)c1)c1cc2oc(=O)c(C(=O)O)cc2s1] | 2-(1-methyl-3-(3- (trifluoromethoxy)phenyl) ureido)-5-oxo-5H-thieno[3,2- b]pyran-6-carboxylic acid | NT |
| 2.088 | 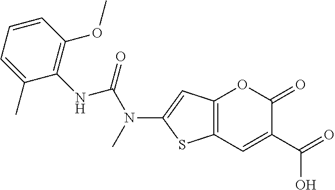 [SMILES: COc1cccc(C)c1NC(=O)N(C)c1cc2oc(=O)c(C(=O)O)cc2s1] | 2-(3-(2-methoxy-6- methylphenyl)-1- methylureido)-5-oxo-5H- thieno[3,2-b]pyran-6- carboxylic acid | NT |
| 2.089 | 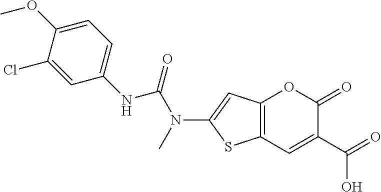 [SMILES: COc1ccc(NC(=O)N(C)c2cc3oc(=O)c(C(=O)O)cc3s2)cc1Cl] | 2-(3-(3-chloro-4- methoxyphenyl)-l - methylureido)-5-oxo-5H- thieno[3,2-b]pyran-6- carboxylic acid | NT |
| 2.090 | 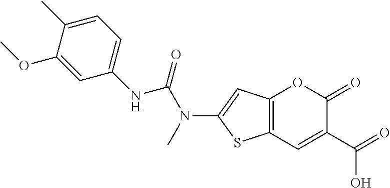 [SMILES: COc1cc(NC(=O)N(C)c2cc3oc(=O)c(C(=O)O)cc3s2)ccc1C] | 2-(3-(3-methoxy-4- methylphenyl)-1- methylureido)-5-oxo-5H- thieno[3,2-b]pyran-6- carboxylic acid | NT |
| 2.091 | 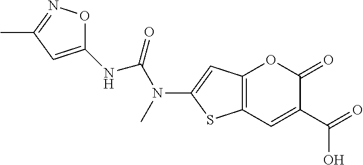 [SMILES: Cc1cc(NC(=O)N(C)c2cc3oc(=O)c(C(=O)O)cc3s2)on1] | 2-(1-methyl-3-(3- methylisoxazol-5- yl)ureido)-5-oxo-5H- thieno[3,2-b]pyran-6- carboxylic acid | NT |
| 2.092 | 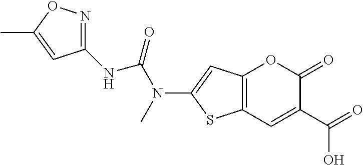 [SMILES: Cc1cc(NC(=O)N(C)c2cc3oc(=O)c(C(=O)O)cc3s2)no1] | 2-(1-methyl-3-(5- methylisoxazol-3- yl)ureido)-5-oxo-5H- thieno[3,2-b]pyran-6- carboxylic acid | NT |
| 2.093 | 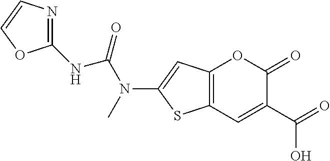 [SMILES: CN(C(=O)Nc1ncco1)c1cc2oc(=O)c(C(=O)O)cc2s1] | 2-(1-methyl-3-(oxazol-2- yl)ureido)-5-oxo-5H- thieno[3,2-b]pyran-6- carboxylic acid | NT |
| 2.094 | 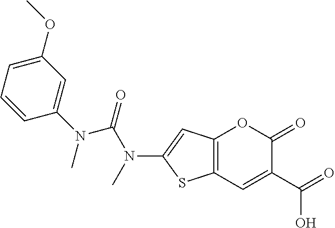 [SMILES: COc1cccc(N(C)C(=O)N(C)c2cc3oc(=O)c(C(=O)O)cc3s2)c1] | 2-(3-(3-methoxyphenyl)- 1,3-dimethylureido)-5-oxo- 5H-thieno[3,2-b]pyran-6- carboxylic acid | NT |
| 2.095 | 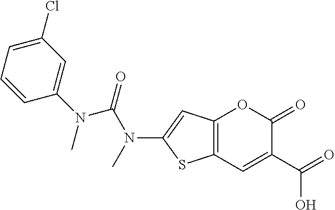 [SMILES: CN(C(=O)N(C)c1cc2oc(=O)c(C(=O)O)cc2s1)c1cccc(Cl)c1] | 2-(3-(3-chlorophenyl)-1,3- dimethylureido)-5-oxo-5H- thieno[3,2-b]pyran-6- carboxylic acid | NT |
| 2.096 | 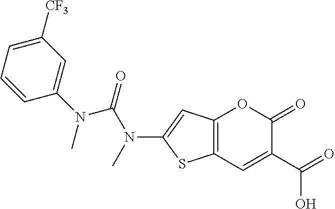 [SMILES: CN(C(=O)N(C)c1cc2oc(=O)c(C(=O)O)cc2s1)c1cccc(C(F)(F)F)c1] | 2-(1,3-dimethyl-3-(3- (trifluoromethyl)phenyl) ureido)-5-oxo-5H-thieno[3,2- b]pyran-6-carboxylic acid | NT |
| 2.097 | 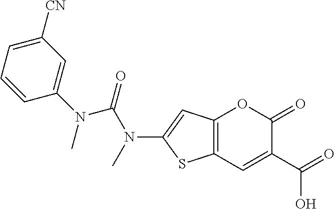 [SMILES: CN(C(=O)N(C)c1cc2oc(=O)c(C(=O)O)cc2s1)c1cccc(C#N)c1] | 2-(3-(3-cyanophenyl)-1,3- dimethylureido)-5-oxo-5H- thieno[3,2-b]pyran-6- carboxylic acid | NT |
| 2.098 | 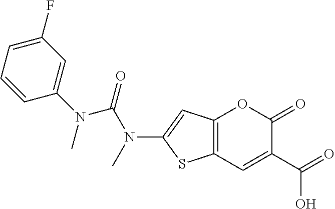 [SMILES: CN(C(=O)N(C)c1cc2oc(=O)c(C(=O)O)cc2s1)c1cccc(F)c1] | 2-(3-(3-fluorophenyl)-1,3- dimethylureido)-5-oxo-5H- thieno[3,2-b]pyran-6- carboxylic acid | NT |
| 2.099 | 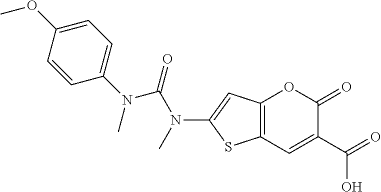 [SMILES: COc1ccc(N(C)C(=O)N(C)c2cc3oc(=O)c(C(=O)O)cc3s2)cc1] | 2-(3-(4-methoxyphenyl)- 1,3-dimethylureido)-5-oxo- 5H-thieno[3,2-b]pyran-6- carboxylic acid | NT |
| 2.100 | 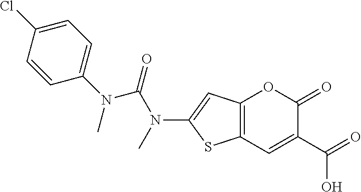 [SMILES: CN(C(=O)N(C)c1cc2oc(=O)c(C(=O)O)cc2s1)c1ccc(Cl)cc1] | 2-(3-(4-chlorophenyl)-1,3- dimethylureido)-5-oxo-5H- thieno[3,2-b]pyran-6- carboxylic acid | NT |
| 2.101 | 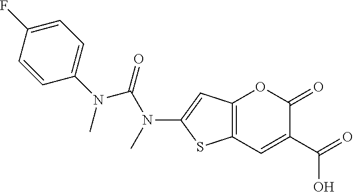 [SMILES: CN(C(=O)N(C)c1cc2oc(=O)c(C(=O)O)cc2s1)c1ccc(F)cc1] | 2-(3-(4-fluorophenyl)-1,3- dimethylureido)-5-oxo-5H- thieno[3,2-b]pyran-6- carboxylic acid | NT |
| 2.102 | 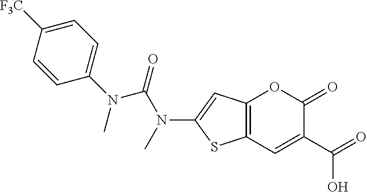 [SMILES: CN(C(=O)N(C)c1cc2oc(=O)c(C(=O)O)cc2s1)c1ccc(C(F)(F)F)cc1] | 2-(1,3-dimethyl-3-(4- (trifluoromethyl)phenyl) ureido)-5-oxo-5H-thieno[3,2- b]pyran-6-carboxylic acid | NT |
| 2.103 | 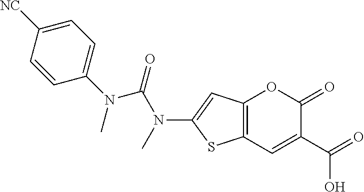 [SMILES: CN(C(=O)N(C)c1cc2oc(=O)c(C(=O)O)cc2s1)c1ccc(C#N)cc1] | 2-(3-(4-cyanophenyl)-1,3- dimethylureido)-5-oxo-5H- thieno[3,2-b]pyran-6- carboxylic acid | NT |
| 2.104 | 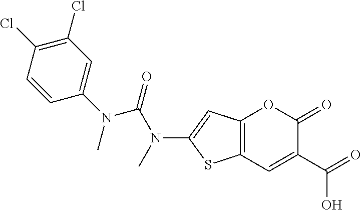 [SMILES: CN(C(=O)N(C)c1cc2oc(=O)c(C(=O)O)cc2s1)c1ccc(Cl)c(Cl)c1] | 2-(3-(3,4-dichlorophenyl)- 1,3-dimethylureido)-5-oxo- 5H-thieno[3,2-b]pyran-6- carboxylic acid | NT |
| 2.105 | 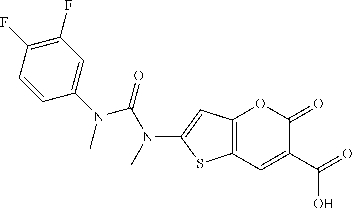 [SMILES: CN(C(=O)N(C)c1cc2oc(=O)c(C(=O)O)cc2s1)c1ccc(F)c(F)c1] | 2-(3-(3,4-difluorophenyl)- 1,3-dimethylureido)-5-oxo- 5H-thieno[3,2-b]pyran-6- carboxylic acid | NT |
| 2.106 | 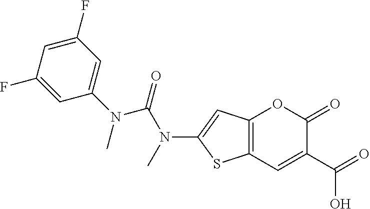 [SMILES: CN(C(=O)N(C)c1cc2oc(=O)c(C(=O)O)cc2s1)c1cc(F)cc(F)c1] | 2-(3-(3,5-difluorophenyl)- 1,3-dimethylureido)-5-oxo- 5H-thieno[3,2-b]pyran-6- carboxylic acid | NT |
| 2.107 | 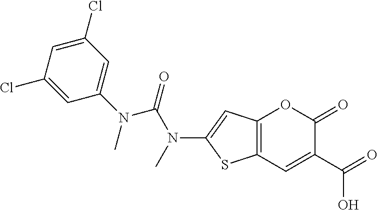 [SMILES: CN(C(=O)N(C)c1cc2oc(=O)c(C(=O)O)cc2s1)c1cc(Cl)cc(Cl)c1] | 2-(3-(3,5-dichlorophenyl)- 1,3-dimethylureido)-5-oxo- 5H-thieno[3,2-b]pyran-6- carboxylic acid | NT |
| 2.108 | 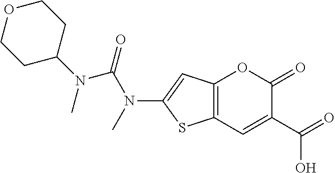 [SMILES: CN(C(=O)N(C)C1CCOCC1)c1cc2oc(=O)c(C(=O)O)cc2s1] | 2-(1,3-dimethyl-3- (tetrahydro-2H-pyran-4- yl)ureido)-5-oxo-5H- thieno[3,2-b]pyran-6- carboxylic acid | NT |
| 2.109 | 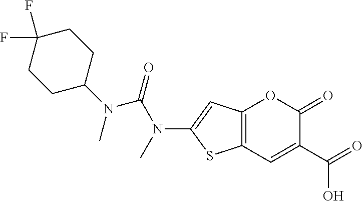 [SMILES: CN(C(=O)N(C)C1CCC(F)(F)CC1)c1cc2oc(=O)c(C(=O)O)cc2s1] | 2-(3-(4,4- difluorocyclohexyl)-1,3- dimethylureido)-5-oxo-5H- thieno[3,2-b]pyran-6- carboxylic acid | NT |
| 2.110 | 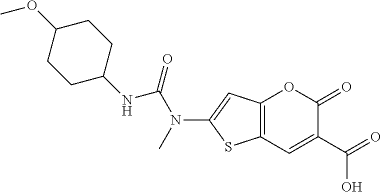 [SMILES: COC1CCC(NC(=O)N(C)c2cc3oc(=O)c(C(=O)O)cc3s2)CC1] | 2-(3-(4- methoxycyclohexyl)-1- methylureido)-5-oxo-5H- thieno[3,2-b]pyran-6- carboxylic acid | NT |
| 2.111 | 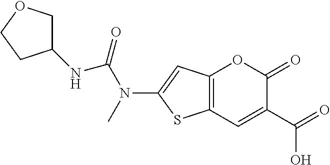 [SMILES: CN(C(=O)NC1CCOC1)c1cc2oc(=O)c(C(=O)O)cc2s1] | 2-(1-methyl-3- (tetrahy drofuran-3- yl)ureido)-5-oxo-5H- thieno[3,2-b]pyran-6- carboxylic acid | NT |

### Example 2: Lactate Detection Assay in Cancer Cell Lines

The inhibition of monocarboxylate transporters of the invention was measured and data are shown in Table II. Cells are maintained in their appropriate growth medium (RPMI medium with 2 g/L glucose, 2 mM L-glutamine supplemented with 10% FBS and P/S (growth medium). 15,000-25,000 cells were seeded into white 96-well plates in growth medium and incubated for 24 hours at 37° C. and 5% CO₂. A duplicate plate was also seeded for normalization by an MTS assay. Dry weight compound stocks were dissolved to a concentration of 10 mM in 100% DMSO. Compounds were further diluted in the assay medium (Lactate media: 10 mM lactate, 5% FBS, and 1× P/S; Glucose media: RPMI, 5% FBS, and 1× P/S). Growth media was changed 24 hours later to assay medium containing 10 μM compound or vehicle (DMSO) control and incubated for 24 hours. Conditioned media was collected and the cells were washed in 200 μL ice-cold PBS. Cells were lysed in 37.5 μL Inactivation solution (25 μL PBS+12.5 μL 0.6N HCl; 0.25% DTAB) which rapidly inhibits cell metabolism, destroys reduced NAD(P)H dinucleotides and inhibits activity of endogenous proteins. After a 5 minute incubation, 12.5 μL Neutralization solution (1M Trizma) is incubated for 1 minute. Intracellular lactate measurements were performed using a Lactate-glo kit (Promega). Briefly, the lactate detection reagent is mixed immediately before use and 50 μL is pipetted into each well and incubated at room temperature for 1 hour. Lactate is oxidized by enzymatic reactions to generate light. The luminescence is recorded using a Spectramax M5e plate reader and the concentration of lactate is determined using a known concentration of spiked lactate in PBS using GraphPad Prism.

Assay data of selected compounds are listed in Table 3, where IC₅₀: A=<1 uM.

TABLE 3
Lactate Detection Assay Results

| | | Lactate Detection Assay |
|---|---|---|
| ID | Structure | (IC₅₀: μM) |
| 1.001 | 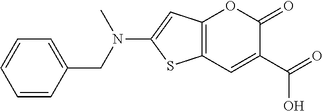 [SMILES: CN(Cc1ccccc1)c1cc2oc(=O)c(C(=O)O)cc2s1] | A |
| 1.002 | 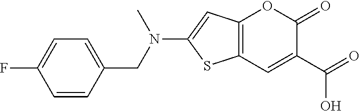 [SMILES: CN(Cc1ccc(F)cc1)c1cc2oc(=O)c(C(=O)O)cc2s1] | A |
| 1.009 | 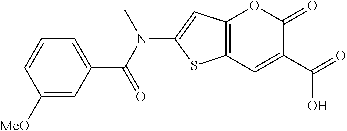 [SMILES: COc1cccc(C(=O)N(C)c2cc3oc(=O)c(C(=O)O)cc3s2)c1] | A |
| 1.017 | 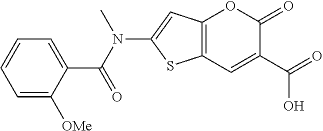 [SMILES: COc1ccccc1C(=O)N(C)c1cc2oc(=O)c(C(=O)O)cc2s1] | A |
| 1.094 | 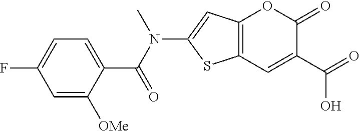 [SMILES: COc1cc(F)ccc1C(=O)N(C)c1cc2oc(=O)c(C(=O)O)cc2s1] | A |
| 2.007 | 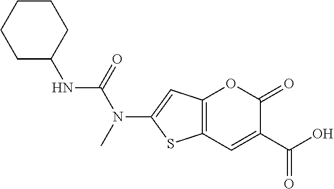 [SMILES: CN(C(=O)NC1CCCCC1)c1cc2oc(=O)c(C(=O)O)cc2s1] | A |
| 2.010 | 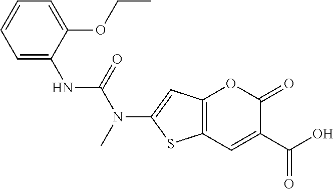 [SMILES: CCOc1ccccc1NC(=O)N(C)c1cc2oc(=O)c(C(=O)O)cc2s1] | A |

### Example 3: In Vivo Treatment of Tumors

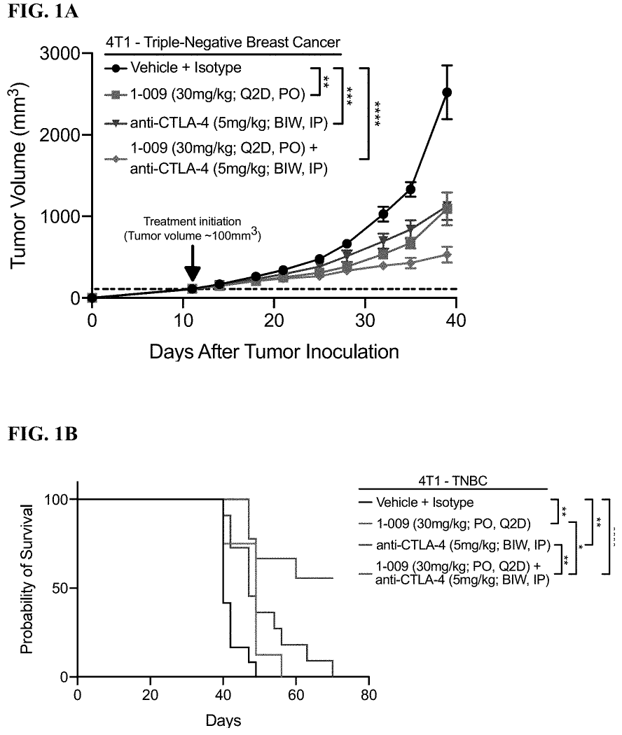

FIGS. 1A-1B: 1×10⁶ 4 T1 tumor cells in 100 μL RPMI-1640 were injected into the 4^{th} inguinal mammary fat pad of 8-10 week old Balb/C mice. Tumor volume as measured using digital calipers and the mice were randomized using block randomization based on tumor volume (˜100 mm³). Treatment was initiated on day 11 and treatment was administered to the following groups: Vehicle (Q2D, PO)+isotype (BIW; IP, 6 doses total); 1.009 (30 mg/kg; Q2D, PO); anti-CTLA-4 (5 mg/kg; BIW, IP, 6 doses total); 1.009 (30 mg/kg; Q2D, PO)+anti-CTLA-4 (5 mg/kg; BIW, IP, 6 doses total). FIG. 1A: Growth kinetics of 4T1 tumors with the respective treatments at Day 39 of tumor growth. One-way ANOVA with Tukey's multiple comparisons test with was used to compare treatment groups on Day 39. Data is represented by the mean+/−SEM. FIG. 1B: Kaplan-Meier Survival plot of 4T1 tumor growth. Mice were considered at endpoint when the tumor reached 2000 mm³. Log-rank (Mantel-Cox) test was used to compare survival curves. Data is represented by the mean+/−SEM. Statistical tests were performed using GraphPad Prism software; ** p<0.005, *** p<0.0005, **** p<0.0001

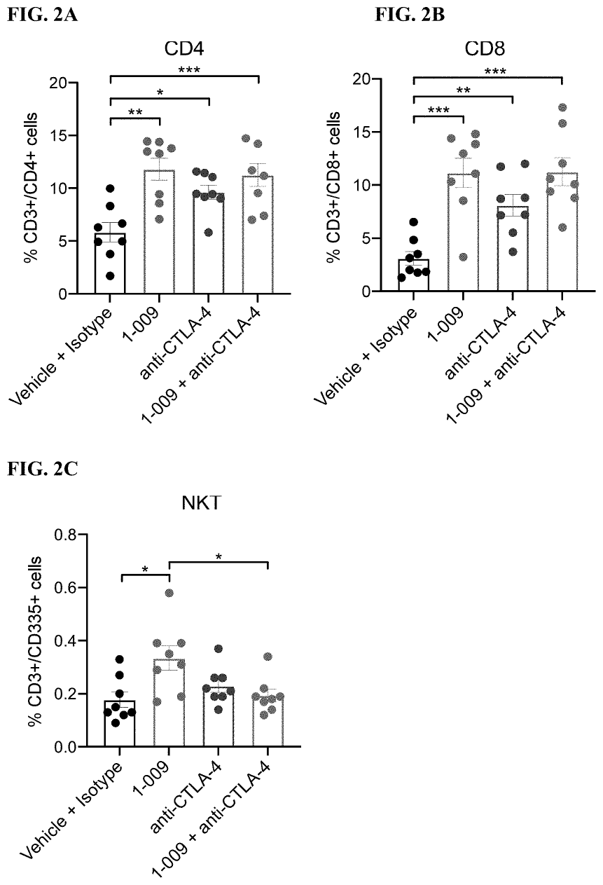

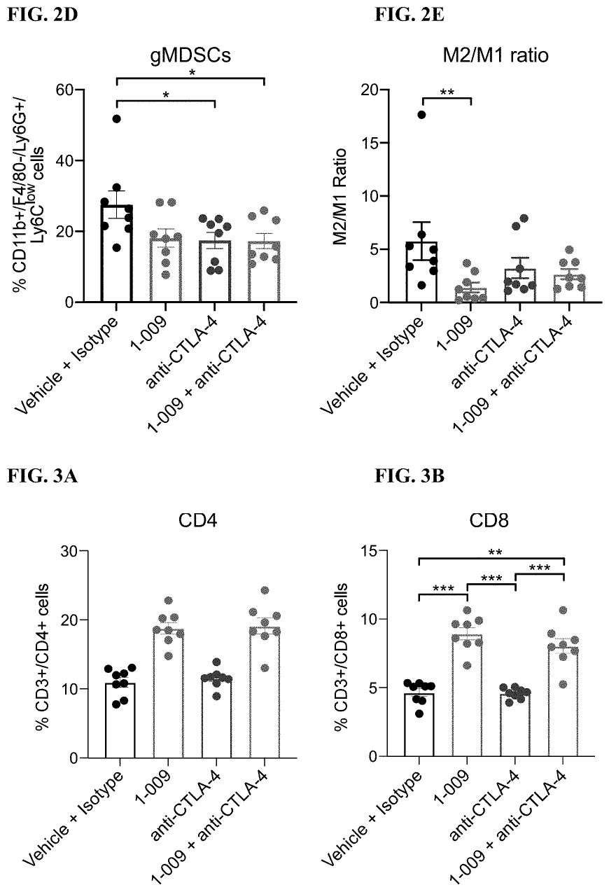

FIGS. 2A-2E: 300,000 4T1 tumor cells in 50 tit RPMI were injected into the 4^{th} inguinal fat pad of 8-10 weeks old Balb/C mice. Tumor volume was measured using digital calipers and the mice were randomized when tumor volume ˜100 mm³. Treatment was administered to the following groups: Vehicle (Q2D, PO)+isotype (BIW; IP); 1.009 (30 mg/kg; QD, PO); anti-CTLA-4 (5 mg/kg; BIW, IP); 1.009 (30 mg/kg; Q2D, PO)+anti-CTLA-4 (5 mg/kg; BIW, IP). Mice were taken down on Day 13 post treatment initiation and tumors were prepared for flow cytometry. Data was acquired using a BD LSRFortessa X-20 flow cytometer and was analyzed with Kaluza software. Data is represented by the mean+/−SEM. * p<0.05, *** p<0.0005, **** p<0.0001

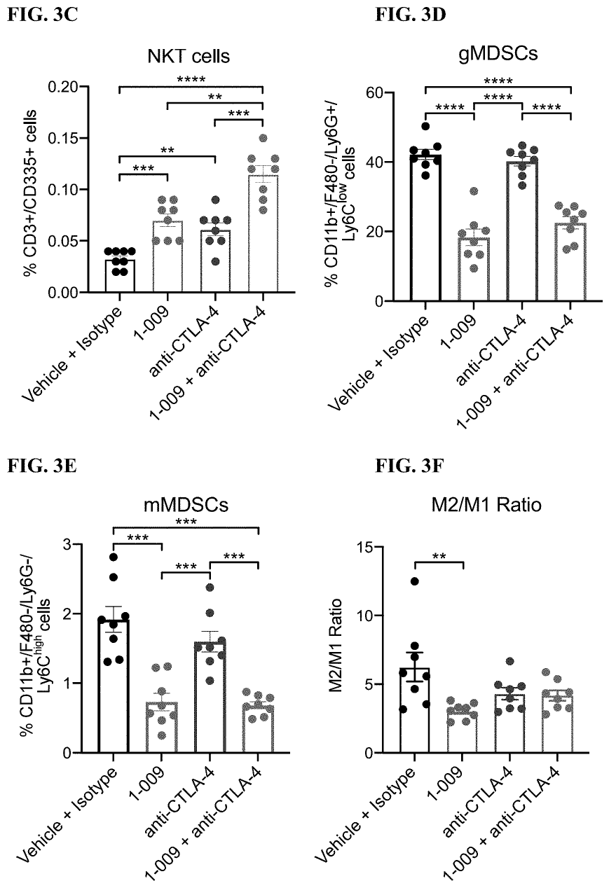

FIGS. 3A-3F: 300,000 4T1 tumor cells in 50 tit RPMI were injected into the 4^{th} inguinal fat pad of 8-10 weeks old Balb/C mice. Tumor volume as measured using digital calipers and the mice were randomized when tumor volume ˜100 mm³. Treatment was administered to the following groups: Vehicle (Q2D, PO)+isotype (BIW; IP); 1.009 (30 mg/kg; QD, PO); anti-CTLA-4 (5 mg/kg; BIW, IP); 1.009 (30 mg/kg; Q2D, PO)+anti-CTLA-4 (5 mg/kg; BIW, IP). Mice were taken down on Day 13 post treatment initiation and tumors were prepared for flow cytometry. Data was acquired using a BD LSRFortessa X-20 flow cytometer and the data was analyzed with Kaluza software. Data is represented by the mean+/−SEM. * p<0.05, ** p<0.005, *** p<0.0005, **** p<0.0001

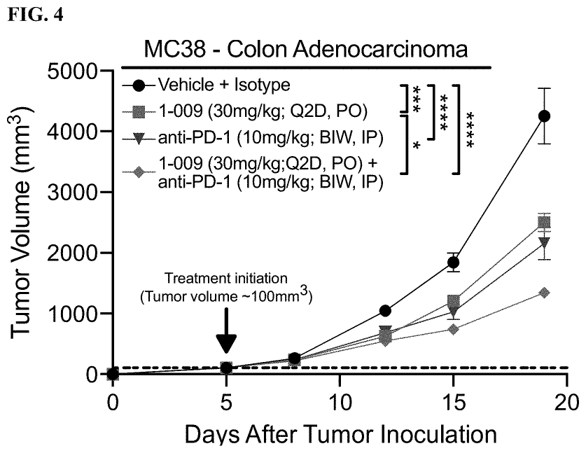

FIG. 4: 1×10⁶ MC38 tumor cells in 100 μL DMEM were injected subcutaneously at the right flank of 8-10 week old C57Bl/6mice. Tumor volume as measured using digital calipers and the mice were randomized using block randomization based on tumor volume (˜100 mm³). Treatment was initiated on day 5 and treatment was administered to the following groups: Vehicle (Q2D, PO)+isotype (BIW; IP, 6 doses total); 1.009 (30 mg/kg; Q2D, PO); anti-PD-1 (10 mg/kg; BIW, IP, 6 doses total); 1.009 (30 mg/kg; Q2D, PO)+anti-PD-1 (10 mg/kg; BIW, IP, 6 doses total). A) Growth kinetics of MC38 tumors with the respective treatments at Day 19 of tumor growth. One-way ANOVA with Tukey's multiple comparisons test with was used to compare treatment groups on Day 19. Data is represented by the mean+/−SEM. Statistical tests were performed using GraphPad Prism software; * p<0.05, *** p<0.0005, **** p<0.0001

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A method of treating cancer in a subject, comprising administering to the subject:
a) a therapeutically effective amount of a monocarboxylate transporter (MCT) inhibitor alone; or in combination with
b) a therapeutically effective amount of an immunotherapy agent,
wherein the monocarboxylate transporter inhibitor is represented by formula (I): 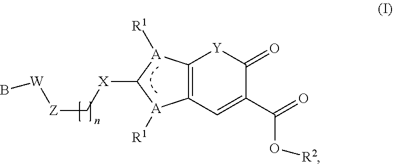
or a pharmaceutically acceptable salt thereof, wherein:
subscript n is 0, 1, or 2;
W is a bond, O, NH, or NR″;
X is O or NR″;
Y is O or NR″;
Z is a bond, CH₂, C═O, SO₂; 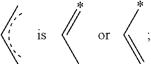
each A is independently selected from the group consisting of N, NR″, S, O, CR″ and CHR″;
each R¹ is independently absent or selected from the group consisting of hydrogen, halogen, C₁₋₆ alkyl, CHF₂, CF₃, CN, —C(O)R″, —C(O)OR″, —SO₂R″, —C(O)NR″2, —C(O)N(OR″)R″, and —C≡CH;
R² is selected from the group consisting of:
hydrogen;
—C(O)R″;
—(CH₂)₀₋₄C(O)R″;
—(CH₂)₀₋₄C(O)OR″;
optionally substituted C₁-6 alkyl;
an optionally substituted 3-8 membered saturated or partially unsaturated cycloalkyl ring;
an optionally substituted 3-8 membered saturated or partially unsaturated heterocycloalkyl ring having 1-2 heteroatoms independently selected from nitrogen, oxygen, and sulfur;
optionally substituted phenyl; and
an optionally substituted 5-6 membered heteroaryl ring having 1-4 heteroatoms independently selected from nitrogen, oxygen, and sulfur;
B is a ring selected from the group consisting of:
a 3-8 membered saturated or partially unsaturated monocyclic cycloalkyl ring, phenyl,
a 8-10 membered bicyclic aryl ring,
a 3-8 membered saturated or partially unsaturated heterocycloalkyl ring having 1-2 heteroatoms independently selected from nitrogen, oxygen, and sulfur,
a 5-6 membered monocyclic heteroaryl ring having 1-4 heteroatoms independently selected from nitrogen, oxygen, and sulfur, and
a 8-10 membered bicyclic heteroaryl ring having 1-4 heteroatoms independently selected from nitrogen, oxygen, and sulfur,
wherein B is optionally substituted with one or more substituents selected from R¹, R′, and R″;
R′ is selected from the group consisting of OH, C₁₋₆ haloalkyl, C₁₋₆ alkoxy, C₁₋₆ haloalkoxy, and O-phenyl optionally substituted with halogen, C₁₋₆ alkyl, or C₁₋₆ alkoxy;
R″ is selected from the group consisting of hydrogen, C₁₋₆ alkyl, and C₁₋₆ haloalkyl; or selected from the group consisting of:
a 3-8 membered saturated or partially unsaturated cycloalkyl ring, optionally substituted with halogen or C₁₋₆ alkyl;
a 3-8 membered saturated or partially unsaturated heterocycloalkyl ring having 1-2 heteroatoms independently selected from nitrogen, oxygen, and sulfur, said ring optionally substituted with halogen or C₁₋₆ alkyl;
phenyl optionally substituted with halogen, C₁₋₆ alkyl, or C₁₋₆ alkoxy; and
a 5-6 membered heteroaryl ring having 1-4 heteroatoms independently selected from nitrogen, oxygen, and sulfur, said ring optionally substituted with halogen or C₁₋₆ alkyl.

2. The method of any one of claim 1, wherein the immunotherapy agent is an immune checkpoint inhibitor, a chimeric antigen receptor (CAR) therapy agent, a vaccine, a modulator of myeloid cells or a macrophage, a modulator of NK cells, a modulator of one or more cytokines, or combinations thereof.

3. The method of claim 2, wherein the immunotherapy agent is an immune checkpoint inhibitor.

4. The method of claim 3, wherein the immune checkpoint inhibitor is a PD-1/PD-L1 inhibitor or a CTLA-4 inhibitor.

5. The method of claim 3, wherein the immune checkpoint inhibitor is pembrolizumab, nivolumab, atezolizumab, durvalumab, avelumab, ipilimumab, cemiplimab-rwlc, camrelizumab, JS001, sintilimab, prolgolimab, tislelizumab, balstilimab, dostarlimab, or retifanlimab.

6. The method of claim 2, wherein the immunotherapy agent is a chimeric antigen receptor (CAR) therapy agent comprising CAR T-cells, CAR-macrophages, CAR-NK cells, or combinations thereof.

7. The method of claim 6, wherein the CAR therapy agent is brexucabtagene, tisagenlecleucel, or axicabtagene ciloleucel.

8. The method of claim 2, wherein the immunotherapy agent is a modulator of myeloid cells or a macrophage, wherein the modulator or macrophage increases M1 macrophages and/or decreases M2 macrophages; or the modulator or macrophage decreases myeloid-derived suppressor cells and/or dendritic cells, or the immunotherapy agent is a modulator of NK cells.

9. The method of claim 2, wherein the immunotherapy agent is a modulator of one or more cytokines, wherein the modulator downregulates one or more of TGFbeta, IL-10, and Arg-1; or the modulator upregulates one or more of TNFalpha, IL-1beta, and IFN-gamma.

10. The method of claim 1, wherein the MCT inhibitor blocks an expression of one or more immune checkpoint molecules.

11. The method of claim 10, wherein the one or more immune checkpoint molecules comprise PD-1, CTLA-4, TIM-3, LAG-3, B7-H3, B7-H4, PD-L1, PD-L2, or combinations thereof in B7 protein family.

12. The method of claim 11, wherein the one or more immune checkpoint molecules further comprise ICOSL, OX40L, Galactin-9, or combinations thereof.

13. The method of claim 1, wherein the MCT inhibitor upregulates a production of one of more of IFN-gamma, TNF-alpha, and IL-1beta.

14. The method of claim 1, wherein the cancer comprises a solid tumor.

15. The method of claim 1, wherein the cancer is breast, melanoma, colorectal, lung, bladder, ovarian, cervical, brain, CNS, skin, pancreatic, gastrointestinal, liver, kidney, head and neck, prostate, osteosarcoma, or combinations thereof.

16-23. (canceled)

24. The method of claim 1, wherein the MCT inhibitor and/or the immunotherapy agent are administered orally.

25. The method of claim 1, wherein the subject is human.

26. (canceled)

27. The method of claim 1, wherein the MCT inhibitor is selected from the group consisting of:

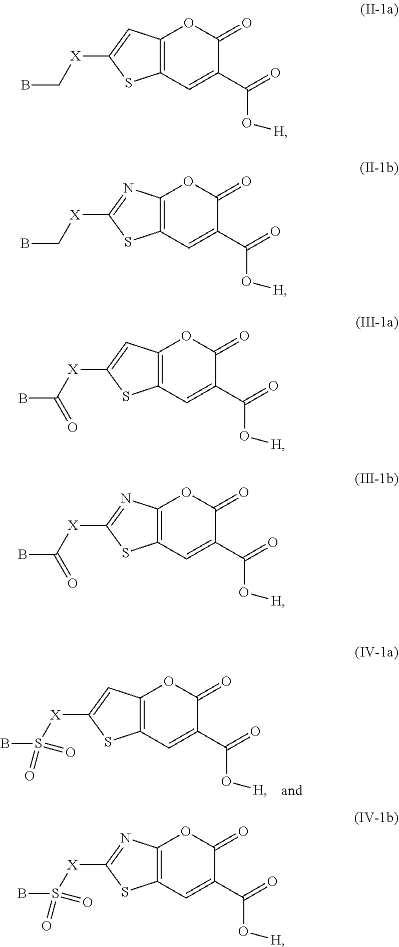

28. The method of 27, wherein X is O, NH or NMe.

29. The method of claim 27, wherein B is selected from the group consisting of:

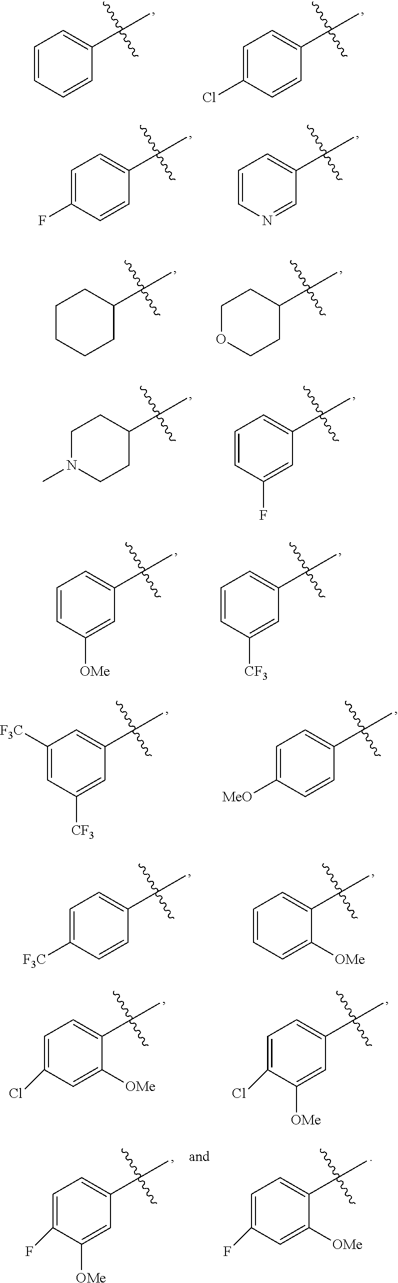

30. The method of claim 1, wherein the MCT inhibitor is selected from the group consisting of:

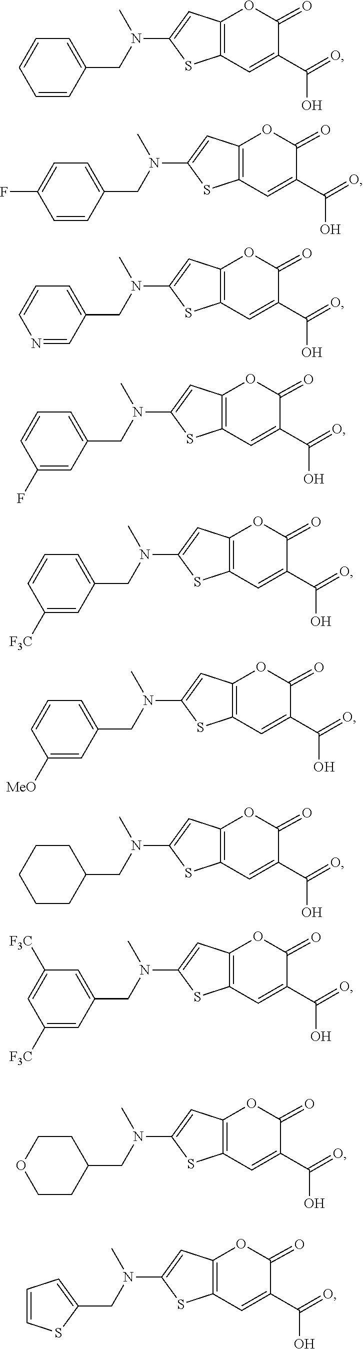

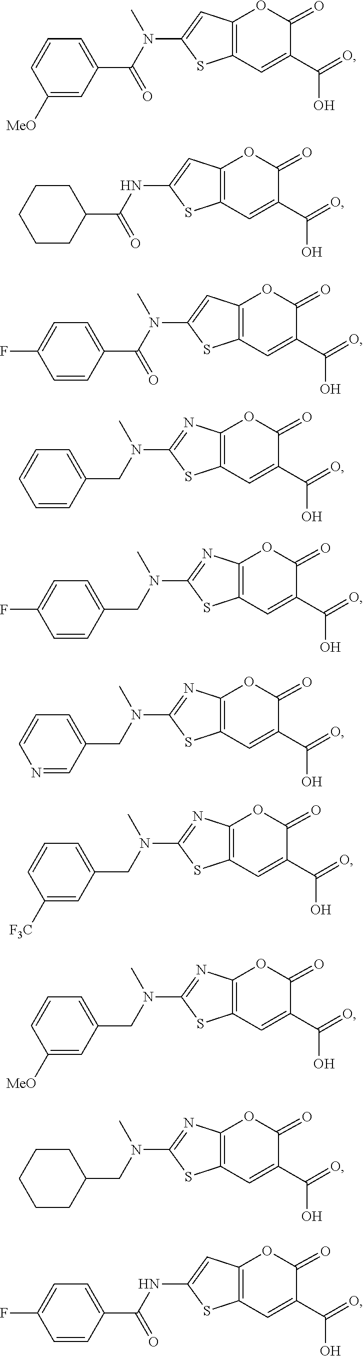

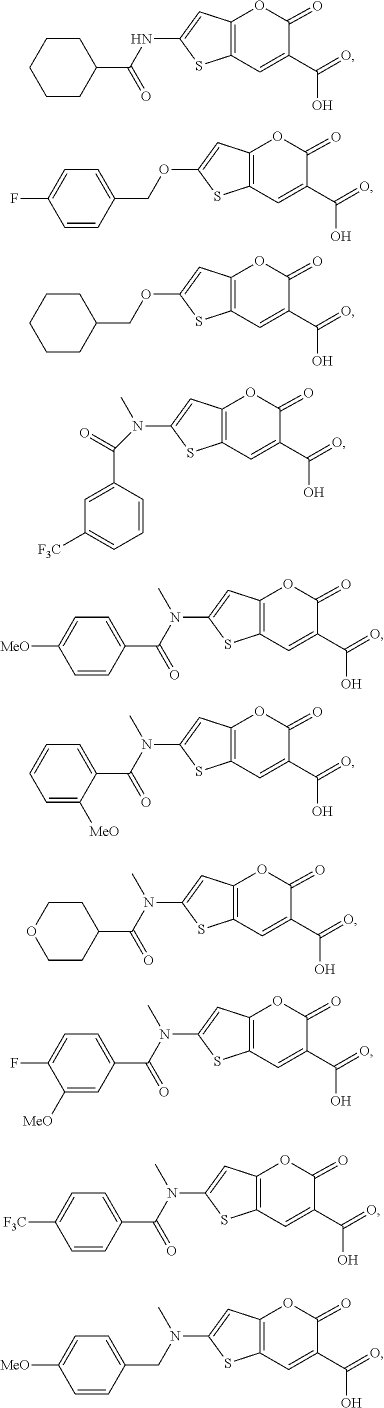

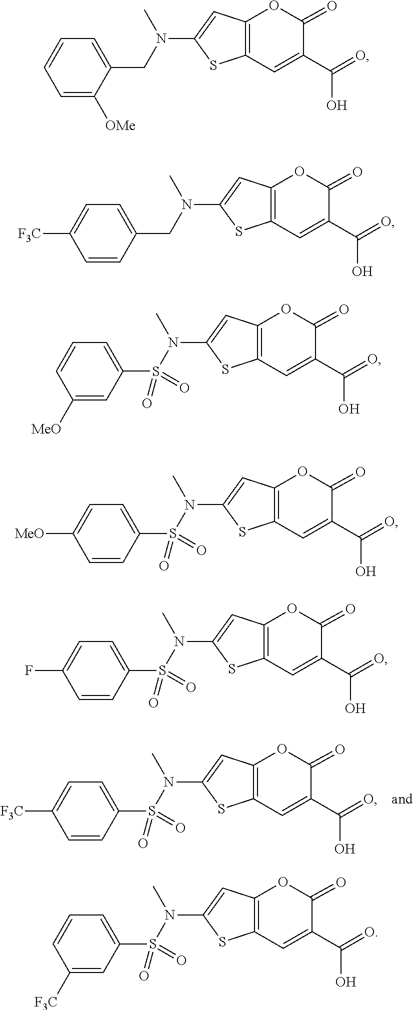

31. The method of claim 30, wherein the MCT inhibitor is selected from the group consisting of:

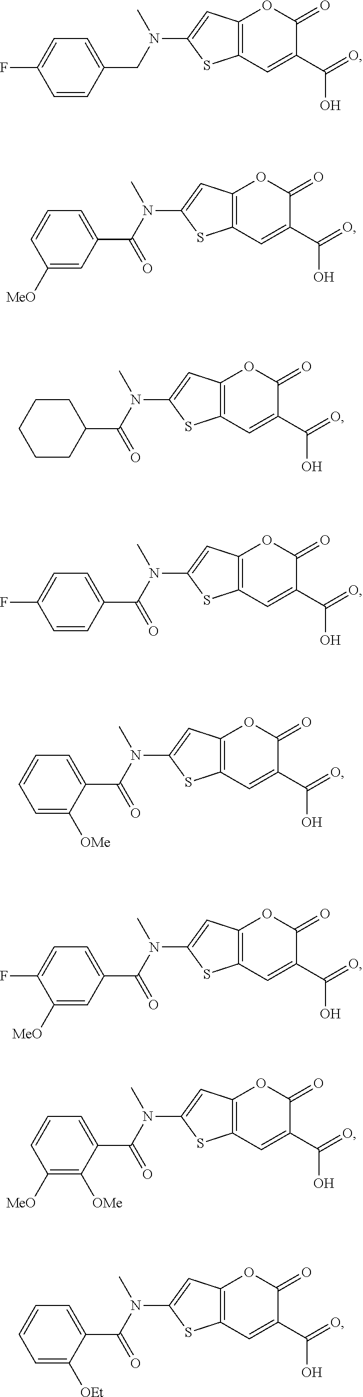

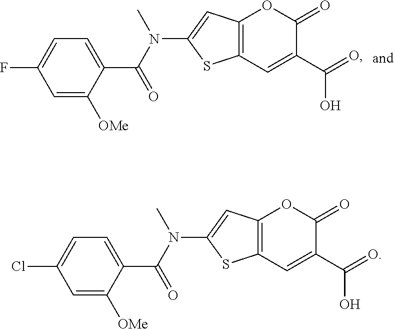 [SMILES: COc1cc(F)ccc1C(=O)N(C)c1cc2oc(=O)c(C(=O)O)cc2s1 ; COc1cc(Cl)ccc1C(=O)N(C)c1cc2oc(=O)c(C(=O)O)cc2s1]

32. The method of claim 1, wherein W is O, NH, or NR″.

33. The method of claim 32, wherein the MCT inhibitor is selected from the group consisting of:

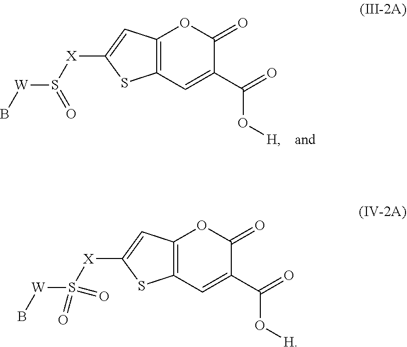

34-39. (canceled)

40. A pharmaceutical composition for treating cancer in a subject, comprising:
a) a therapeutically effective amount of a monocarboxylate transporter (MCT) inhibitor of formula (I) according to claim 1; and
b) a therapeutically effective amount of an immune therapy agent according to claim 2,
together with a pharmaceutically acceptable carrier or excipient.

41. A kit for treating cancer in a subject, comprising:
a) a therapeutically effective amount of a monocarboxylate transporter (MCT) inhibitor of formula (I) according to claim 1; and
b) a therapeutically effective amount of an immune therapy agent according to claim 2,
with instruction for effective administration.

42-43. (canceled)
